(19) United States
(12) Patent Application Publication
(10) Pub. No.: US 2015/0376587 A1
(43) Pub. Date: Dec. 31, 2015

(54) RNA Modification to Engineer Cas9 Activity

(71) Applicant: Caribou Biosciences, Inc., Berkeley, CA (US)

(72) Inventors: Andrew Paul May, San Francisco, CA (US); Paul Donohoue, Berkeley, CA (US); Christopher Nye, San Jose, CA (US); Euan Slorach, Oakland, CA (US); Rachel Haurwitz, Kensington, CA (US)

(21) Appl. No.: 14/791,200

(22) Filed: Jul. 2, 2015

Related U.S. Application Data
(63) Continuation of application No. PCT/US2015/037546, filed on Jun. 24, 2015.
(60) Provisional application No. 62/017,113, filed on Jun. 25, 2014.
(60) Provisional application No. 62/065,515, filed on Oct. 17, 2014.
(60) Provisional application No. 62/088,277, filed on Dec. 5, 2014.

(51) Int. Cl. C12N 9/22; A61K 35/12; C12N 15/113

(52) U.S. Cl. CPC C12N 9/22; C12N 15/113; A61K 35/12; C12N 2310/531; C12Y 301/00

(57) ABSTRACT

The disclosure provides for compositions, methods and kits, for reducing off-target effects of genome engineering.

### CROSS-REFERENCE

This application is a continuation application of International Application No. PCT/US2015/037546, filed Jun. 24, 2015 [Attorney Docket No. 44287-756.601], which application claims the benefit of U.S. Provisional Application No. 62/017,113, filed Jun. 25, 2014 [Attorney Docket No. 44287-756.101], U.S. Provisional Application No. 62/065,515, filed Oct. 17, 2014 [Attorney Docket No. 44287-756.102], and U.S. Provisional Application No. 62/088,277, filed Dec. 5, 2014 [Attorney Docket No. 44287-756.103], each of which is incorporated herein by reference in its entirety.

### BACKGROUND

Non-natural nucleic acid-targeting nucleic acids can be used in a ribonucleoprotein complex with a site-directed polypeptide, for example, Cas9, to guide the site-directed polypeptide to sequences of interest in a target nucleic acid, for example, DNA. The site-directed polypeptide can target and cut other sequences with similarity to the intended target sequence. There is a need for identifying and engineering nucleic acid-targeting nucleic acids with high specificity for their intended target nucleic acid.

### SUMMARY OF THE INVENTION

In one aspect, a composition is provided comprising an engineered nucleoprotein complex. In some cases, the engineered nucleoprotein complex comprises a Cas9 polypeptide and a non-natural nucleic acid-targeting nucleic acid, wherein the non-natural nucleic acid-targeting nucleic acid comprises an engineered region selected from the group consisting of: an engineered stem loop duplex structure, an engineered bulge region, an engineered hairpin located 3′ of the stem loop duplex structure, and any combination thereof. In some cases, the engineered nucleoprotein complex results in a modification of a target region of a genomic DNA. In some cases, the engineered nucleoprotein complex has a decreased ability to modify a DNA molecule in a region that is not the target region as compared to a control nucleoprotein complex. In some cases, the control nucleoprotein complex comprises a control nucleic acid-targeting nucleic acid that does not comprise an engineered stem loop duplex structure, an engineered bulge region, and an engineered hairpin located 3′ of the stem loop duplex structure.

In another aspect, a composition is provided comprising a cell modified with the engineered nucleoprotein complex described above. In some cases, the cell comprises a eukaryotic cell. In some cases, the cell comprises a stem cell. In some cases, the non-natural nucleic acid-targeting nucleic acid comprises RNA nucleobases. In some cases, the non-natural nucleic acid-targeting nucleic acid is RNA. In some cases, the non-natural nucleic acid-targeting nucleic acid comprises non-natural nucleobases. In some cases, the non-natural nucleic acid-targeting nucleic acid further comprises a covalently linked moiety. In some cases, the non-natural nucleic acid-targeting nucleic acid comprises one or more mutations in the engineered region selected from the group consisting of: an engineered stem loop duplex structure, an engineered bulge region, an engineered hairpin located 3′ of the stem loop duplex structure, and any combination thereof. In some cases, the one or more mutation comprises an insertion of one or more nucleotides. In some cases, the one or more mutation comprises a deletion of one or more nucleotides. In some cases, the one or more mutation comprises a substitution of one or more nucleotides with a non-natural nucleotide. In some cases, the non-natural nucleic acid-targeting nucleic acid comprises two or more mutations and a first mutation is separated by at least one nucleobase from a second mutation. In some cases, the non-natural nucleic acid-targeting nucleic acid comprises two or more mutations and a first mutation is adjacent to a second mutation. In some cases, the engineered nucleoprotein complex has about a 10% decreased ability to modify the DNA in a region that is not the target region as compared to the control nucleoprotein complex. In some cases, the engineered region is an engineered stem loop duplex structure. In some cases, the engineered region is an engineered bulge region. In some cases, the engineered region is an engineered hairpin located 3′ of the stem loop duplex structure. In some cases, the composition further comprises a spacer region located 5′ of the stem loop duplex structure. In some cases, the spacer region comprises between 18 to 21 nucleotides in length, inclusive. In some cases, the modification of the target region of the genomic DNA comprises cleavage of a phosphodiester bond.

In another aspect, a kit is provided comprising the composition described above and a suitable buffer. In some cases, the kit further comprises instructions for use.

In another aspect, a pharmaceutical composition is provided comprising the cell modified with the engineered nucleoprotein complex described above. In some cases, the pharmaceutical composition further comprises an excipient.

In another aspect, a composition is provided comprising a genomic DNA, wherein the genomic DNA comprises a target region, a Cas9 polypeptide, and a non-natural nucleic acid-targeting nucleic acid comprising a spacer extension. In some cases, the non-natural nucleic acid-targeting nucleic acid has a decreased ability to modify the genomic DNA in regions that are not the target region as compared to a control nucleic acid-targeting nucleic acid. In some cases, the control nucleic acid-targeting nucleic acid does not comprise a spacer extension.

In another aspect, a composition is provided comprising a cell modified with the composition described above. In some cases, the cell comprises a eukaryotic cell. In some cases, the cell comprises a stem cell. In some cases, the non-natural nucleic acid-targeting nucleic acid comprises RNA nucleobases. In some cases, the non-natural nucleic acid-targeting nucleic acid is RNA. In some cases, the non-natural nucleic acid-targeting nucleic acid comprises non-natural nucleobases. In some cases, the non-natural nucleic acid-targeting nucleic acid further comprises a covalently linked moiety. In some cases, the non-natural nucleic acid-targeting nucleic acid comprises two or more mutations and a first mutation is separated by at least one nucleobase from a second mutation. In some cases, the non-natural nucleic acid-targeting nucleic acid comprises two or more mutations and a first mutation is adjacent to a second mutation. In some cases, the engineered nucleoprotein complex has about a 10% decreased ability to modify the DNA in a region that is not the target region as compared to the control nucleoprotein complex. In some cases, the composition further comprises a spacer region located 5′ of a stem loop duplex structure in the non-natural nucleic acid-targeting nucleic acid. In some cases, the spacer region comprises between 18 to 21 nucleotides in length, inclusive. In some cases, the modification of the target region of the genomic DNA comprises cleavage of a phosphodiester bond. In some cases, the spacer extension comprises a G. In some cases, the spacer extension comprises an A. In some cases, the spacer extension comprises an U. In some cases, the spacer extension comprises a C. In some cases, the spacer extension comprises one or more 5′ nucleotides. In some cases, the one additional 5′ nucleotide is a G. In some cases, the spacer extension is located 5′ to the spacer region. In some cases, a combined length of the spacer extension and the spacer region is between 20 to 22 nucleotides in length, inclusive.

In another aspect, a kit is provided comprising the composition described above and a suitable buffer. In some cases, the kit further comprises instructions for use. In another aspect, a pharmaceutical composition is provided comprising the cell modified with the composition described above. In some cases, the pharmaceutical composition comprises an excipient.

In one aspect the disclosure provides for a composition comprising: a non-natural CRISPR RNA 5′ spacer extension of a nucleic acid-targeting nucleic acid, wherein the 5′ spacer extension comprises one or more additional 5′ nucleotides. In some embodiments, one of the one or more additional 5′ nucleotides is a guanine. In some embodiments, one of the one or more additional 5′ nucleotides is an adenine. In some embodiments, one of the one or more additional 5′ nucleotides is a cytosine. In some embodiments, one of the one or more additional 5′ nucleotides is a uracil. In some embodiments, the 5′ spacer extension comprises one additional 5′ nucleotide. In some embodiments, the one additional nucleotide is a guanine. In some embodiments, the one additional nucleotide is an adenine. In some embodiments, the one additional nucleotide is a cytosine. In some embodiments, the one additional nucleotide is a uracil. In some embodiments, a spacer region of the nucleic acid-targeting nucleic acid is 21 nucleotides in length. In some embodiments, a spacer region of the nucleic acid-targeting nucleic acid is 20 nucleotides in length. In some embodiments, a spacer region of the nucleic acid-targeting nucleic acid is 19 nucleotides in length. In some embodiments, a spacer region of the nucleic acid-targeting nucleic acid is from 19 to 21 nucleotides in length. In some embodiments, the length of both a spacer region of the nucleic acid-targeting nucleic acid and the 5′ spacer extension is 22 nucleotides in length. In some embodiments, the length of both a spacer region of the nucleic acid-targeting nucleic acid and the 5′ spacer extension is 21 nucleotides in length. In some embodiments, the length of both a spacer region of the nucleic acid-targeting nucleic acid and the 5′ spacer extension is 20 nucleotides in length. In some embodiments, the nucleic acid-targeting nucleic acid is adapted to reduce off-target binding by at least 10% compared to a nucleic acid-targeting nucleic acid lacking the one or more additional 5′ nucleotides. In some embodiments, the nucleic acid-targeting nucleic acid is adapted to reduce off-target binding by at least 20% compared to a nucleic acid-targeting nucleic acid lacking the one or more additional 5′ nucleotides. In some embodiments, the nucleic acid-targeting nucleic acid is adapted to reduce off-target binding by at least 30% compared to a nucleic acid-targeting nucleic acid lacking the one or more additional 5′ nucleotides. In some embodiments, the nucleic acid-targeting nucleic acid is adapted to reduce off-target cleavage by at least 10% compared to a nucleic acid-targeting nucleic acid lacking the one or more additional 5′ nucleotides. In some embodiments, the nucleic acid-targeting nucleic acid is adapted to reduce off-target cleavage by at least 20% compared to a nucleic acid-targeting nucleic acid lacking the one or more additional 5′ nucleotides. In some embodiments, the nucleic acid-targeting nucleic acid is adapted to reduce off-target cleavage by at least 30% compared to a nucleic acid-targeting nucleic acid lacking the one or more additional 5′ nucleotides.

In one aspect, the disclosure provides for a method for reducing binding of a nucleic acid-targeting nucleic acid to an off-target nucleic acid comprising: contacting a complex comprising a site-directed polypeptide and a modified non-natural nucleic acid-targeting nucleic acid to a target nucleic acid, wherein the complex contacts the target nucleic acid at least 10% more than the off-target nucleic acid. In some embodiments, the complex contacts the target nucleic acid at least 20% more than the off-target nucleic acid. In some embodiments, the complex contacts the target nucleic acid at least 30% more than the off-target nucleic acid. In some embodiments, the contacting comprises hybridizing the nucleic acid-targeting nucleic acid to the target nucleic acid. In some embodiments, the hybridizing comprises hybridizing a portion of the nucleic acid-targeting nucleic acid to the target nucleic acid. In some embodiments, the portion of the nucleic acid-targeting nucleic acid comprises a spacer. In some embodiments, the portion of the nucleic acid-targeting nucleic acid comprises a spacer and one or more 5′ additional nucleotides. In some embodiments, the method further comprises modifying the target nucleic acid. In some embodiments, modifying comprises modifying the target nucleic acid at least 10% more than the off-target nucleic acid. In some embodiments, modifying comprises modifying the target nucleic acid at least 20% more than the off-target nucleic acid. In some embodiments, the modifying comprises modifying the target nucleic acid at least 30% more than the off-target nucleic acid. In some embodiments, the modifying comprises cleaving the target nucleic acid. In some embodiments, the modifying comprises deleting nucleotides from the target nucleic acid. In some embodiments, the modifying comprises inserting a donor polynucleotide in the target nucleic acid. In some embodiments, the modifying comprises increasing transcription of the target nucleic acid. In some embodiments, the modifying comprises decreasing transcription of the target nucleic acid.

In one aspect, the disclosure provides for a kit comprising: a composition comprising: a non-natural CRISPR RNA 5′ spacer extension of a nucleic acid-targeting nucleic acid, wherein the 5′ spacer extension comprises one or more additional 5′ nucleotides; and a buffer. In some embodiments, the kit further comprises instructions for use.

In one aspect the disclosure provides for a composition comprising: a non-natural CRISPR RNA spacer of a nucleic acid-targeting nucleic acid, wherein the spacer comprises one or more 5′ nucleotide deletions. In some embodiments, one of the one or more 5′ nucleotide deletions is a guanine. In some embodiments, one of the one or more 5′ nucleotide deletions is an adenine. In some embodiments, one of the one or more 5′ nucleotide deletions is a cytosine. In some embodiments, one of the one or more 5′ nucleotide deletions is a uracil. In some embodiments, the spacer comprises one 5′ nucleotide deletion. In some embodiments, the one 5′ nucleotide deletion is a guanine. In some embodiments, the one 5′ nucleotide deletion is an adenine. In some embodiments, the one 5′ nucleotide deletion is a cytosine. In some embodiments, the one 5′ nucleotide deletion is a uracil. In some embodiments, the spacer region of the nucleic acid-targeting nucleic acid is 20 nucleotides in length. In some embodiments, the spacer region of the nucleic acid-targeting nucleic acid is 19 nucleotides in length. In some embodiments, the spacer region of the nucleic acid-targeting nucleic acid is 18 nucleotides in length. In some embodiments, a spacer region of the nucleic acid-targeting nucleic acid is from 18 to 21 nucleotides in length. In some embodiments, the nucleic acid-targeting nucleic acid is adapted to reduce off-target binding by at least 10% compared to a nucleic acid-targeting nucleic acid lacking the one or more 5′ nucleotide deletions. In some embodiments, the nucleic acid-targeting nucleic acid is adapted to reduce off-target binding by at least 20% compared to a nucleic acid-targeting nucleic acid lacking the one or more 5′ nucleotide deletions. In some embodiments, the nucleic acid-targeting nucleic acid is adapted to reduce off-target binding by at least 30% compared to a nucleic acid-targeting nucleic acid lacking the one or more 5′ nucleotide deletions. In some embodiments, the nucleic acid-targeting nucleic acid is adapted to reduce off-target cleavage by at least 10% compared to a nucleic acid-targeting nucleic acid lacking the one or more 5′ nucleotide deletions. In some embodiments, the nucleic acid-targeting nucleic acid is adapted to reduce off-target cleavage by at least 20% compared to a nucleic acid-targeting nucleic acid lacking the one or more 5′ nucleotide deletions. In some embodiments, the nucleic acid-targeting nucleic acid is adapted to reduce off-target cleavage by at least 30% compared to a nucleic acid-targeting nucleic acid lacking the one or more 5′ nucleotide deletions.

In one aspect the disclosure provides for a method for reducing binding of a nucleic acid-targeting nucleic acid to an off-target nucleic acid comprising: contacting a complex comprising a site-directed polypeptide and a nucleic acid-targeting nucleic acid comprising a non-natural CRISPR RNA spacer, wherein the spacer comprises one or more 5′ nucleotide deletions, to a target nucleic acid, wherein the complex contacts the target nucleic acid at least 10% more than the off-target nucleic acid. In some embodiments, the complex contacts the target nucleic acid at least 20% more than the off-target nucleic acid. In some embodiments, the complex contacts the target nucleic acid at least 30% more than the off-target nucleic acid. In some embodiments, the contacting comprises hybridizing the nucleic acid-targeting nucleic acid to the target nucleic acid. In some embodiments, the hybridizing comprises hybridizing a portion of the nucleic acid-targeting nucleic acid to the target nucleic acid. In some embodiments, the portion of the nucleic acid-targeting nucleic acid comprises a spacer. In some embodiments, the portion of the nucleic acid-targeting nucleic acid comprises a spacer and one or more 5′ additional nucleotides. In some embodiments, the method further comprises modifying the target nucleic acid. In some embodiments, modifying comprises modifying the target nucleic acid at least 20% more than the off-target nucleic acid. In some embodiments, the modifying comprises modifying the target nucleic acid at least 30% more than the off-target nucleic acid. In some embodiments, the modifying comprises cleaving the target nucleic acid. In some embodiments, the modifying comprises deleting nucleotides from the target nucleic acid. In some embodiments, the modifying comprises inserting a donor polynucleotide in the target nucleic acid. In some embodiments, the modifying comprises increasing transcription of the target nucleic acid. In some embodiments, the modifying comprises decreasing transcription of the target nucleic acid.

In one aspect, the disclosure provides for a composition comprising: a nucleic acid-targeting nucleic acid, wherein the nucleic acid-targeting nucleic acid comprises a non-natural CRISPR RNA spacer region and a nexus region, wherein the nexus region comprises a hairpin 3′ of a stem-loop duplex structure, wherein a first strand of the stem-loop duplex comprises at least 50% identity to a CRISPR RNA over 6 contiguous nucleotides, and a second strand of the duplex comprises at least 50% identity to a tracrRNA over 6 contiguous nucleotides. In some embodiments, the hairpin in the nexus region comprises the natural number of base-paired nucleotides in the duplex of the hairpin. In some embodiments, the nexus region is a non-natural nexus region. In some embodiments, the hairpin comprises a dinucleotide duplex. In some embodiments, the hairpin comprises a trinucleotide duplex. In some embodiments, the nexus is located immediately 3′ to the stem-loop duplex. In some embodiments, the nexus is located from 1 to 5 nucleotides 3′ of the stem-loop duplex. In some embodiments, the nucleic acid-targeting nucleic acid comprises a single-stranded region 3′ of the hairpin of the nexus. In some embodiments, the single-stranded region comprises from 1-10 nucleotides. In some embodiments, the single-stranded region comprises from 2 to 6 nucleotides. In some embodiments, the single-stranded region comprises at least 50% of the length of the natural single-stranded region. In some embodiments, the single-stranded region comprises at least 60% of the length of the natural single-stranded region. In some embodiments, the single-stranded region comprises at least 70% of the length of the natural single-stranded region. In some embodiments, the single-stranded region comprises at least 80% of the length of the natural single-stranded region. In some embodiments, the single-stranded region comprises the natural single-stranded region of the nucleic acid-targeting nucleic acid. In some embodiments, the single-stranded region comprises a non-natural single stranded region. In some embodiments, the composition further comprises one or more hairpins 3′ of the single-stranded region.

In yet another aspect, the disclosure provides for a composition comprising: a nucleic acid-targeting nucleic acid, wherein the nucleic acid-targeting nucleic acid comprises a non-natural CRISPR RNA spacer region and a nexus region, wherein the nexus region comprises a hairpin 3′ of a stem-loop duplex structure, wherein the stem-loop duplex structure comprises a sequence that adopts a tertiary structure that can be bound by a Cas9 polypeptide. In some embodiments, the hairpin in the nexus region comprises the natural number of base-paired nucleotides in the duplex of the hairpin. In some embodiments, the nexus region is a non-natural nexus region. In some embodiments, the hairpin comprises a dinucleotide duplex. In some embodiments, the hairpin comprises a trinucleotide duplex. In some embodiments, the nexus is located immediately 3′ to the stem-loop duplex. In some embodiments, the nexus is located from 1 to 5 nucleotides 3′ of the stem-loop duplex. In some embodiments, the nucleic acid-targeting nucleic acid comprises a single-stranded region 3′ of the hairpin of the nexus. In some embodiments, the single-stranded region comprises from 1-10 nucleotides. In some embodiments, the single-stranded region comprises from 2 to 6 nucleotides. In some embodiments, the single-stranded region comprises at least 50% of the length of the natural single-stranded region. In some embodiments, the single-stranded region comprises at least 60% of the length of the natural single-stranded region. In some embodiments, the single-stranded region comprises at least 70% of the length of the natural single-stranded region. In some embodiments, the single-stranded region comprises at least 80% of the length of the natural single-stranded region. In some embodiments, the single-stranded region comprises the natural single-stranded region of the nucleic acid-targeting nucleic acid. In some embodiments, the single-stranded region comprises a non-natural single stranded region. In some embodiments, the composition further comprises one or more hairpins 3′ of the single-stranded region.

### INCORPORATION BY REFERENCE

The subject matter of U.S. application Ser. No. 14/206,319 filed Mar. 12, 2014 [Attorney Docket No. 44287-722.201] is incorporated herein by reference in its entirety.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts an exemplary embodiment of a single guide nucleic acid-targeting nucleic acid of the disclosure.

FIG. 1B depicts an exemplary embodiment of a single guide nucleic acid-targeting nucleic acid of the disclosure.

FIG. 2 depicts an exemplary embodiment of a double guide nucleic acid-targeting nucleic acid of the disclosure.

FIG. 3A-D shows an overview of illustrative variants of a nucleic acid-targeting nucleic acid. FIG. 3A depicts an overview and nomenclature of modules for a single guide RNA (sgRNA) of the Streptococcus pyogenes (S. pyogenes) Cas9. The modules can include for example, spacer region, upper stem, bulge, lower stem, nexus (i.e. a region comprising a hairpin 3′ of the first stem-loop duplex structure), and hairpins. FIG. 3B shows illustrative sgRNA variants (guide RNA variants). Altered modules of the sgRNA are shown, and mutated nucleotides are represented in bold. Biochemical (FIG. 3C) and cell-based T7E1 (FIG. 3D) DNA cleavage assays were performed with each guide RNA variant in combination with the S. pyogenes Cas9. Results are representative of at least three independent experiments.

FIG. 4 shows results of Cas9 cleavage of an AAVS1 DNA fragment measured from cells (HEK-293 T7E1) and biochemically (Biochemical). Upper gel panel shows results of T7E1 assay for indel detection of AAVS1 DNA from HEK293-Cas9 cells transfected with guide RNA variants. Lower panel shows Cas9-guide variant mediated cleavage of the same AAVS target fragment in vitro. Three experimental replicates of guide variants were performed for cell based and biochemical assays (replicates not shown). 100 bp ladder (NEB) serves as marker (left lane).

FIG. 5 shows results of biochemical (top panel) and cell-based T7E1 (bottom panel) DNA cleavage assays performed with illustrative guide RNA variants for the VEGFA GX20 spacer.

FIG. 6 shows results of biochemical (top panel) and cell-based T7E1 (bottom panel) DNA cleavage assays performed with illustrative guide RNA variants for the EMX-1 GX20 spacer.

FIG. 7 shows results of biochemical (top panel) and cell-based T7E1 (bottom panel) DNA cleavage assays performed with illustrative guide RNA variants for the VEGFA GX19 spacer.

FIG. 8 shows results of biochemical (top panel) and cell-based T7E1 (bottom panel) DNA cleavage assays performed with illustrative guide RNA variants for the EMX-1 GX19 spacer.

FIGS. 9A and B show biochemical cleavage of potential off-target sites for EMX-1 and VEGFA GX20 spacers. PCR products were amplified from HEK-293 genomic DNA and cleaved with GX20 sgRNA/Cas9 complexes

FIG. 10A-C shows on- and off-target assays for spacers targeting different human genes (DNMT3A, DNMT3B, CCR5, EMX-1, C4BPB, RNF2, FANCF, and VEGFA) using GX20 sgRNAs.

FIGS. 11A and B shows on- and off-target sequences for targets in human genome (DNMT3A, DNMT3B, CCR5, EMX-1, C4BPB, RNF2, FANCF, and VEGFA).

FIG. 12A-D shows T7E1 assays at on and off-target sites for GX19, GX20 and GGX20 sgRNAs.

FIGS. 13A and B shows AX19, CX19, TX19, GX19, GX20 spacer sequences for EMX-1 and VEGFA sgRNAs. FIG. 13A illustrates that transcription yields are significantly reduced for the AX19, CX19 and TX19 sgRNAs. FIG. 13B illustrates results of biochemical activity (in vitro) and cell-based assay (in vivo). Biochemical activity (in vitro) for each of the sgRNAs shows that activity appears to correlate with sgRNA concentration. Cell-based assays (in vivo) for the same sgRNA show equivalent on-target activity for VEGFA, but not for EMX-1.

FIG. 14 shows a comparison of activity for EMX-1 GX19 and GX20 guide RNA variants at on- and off-target sites. Guide RNA variants do not alter the off-target activity for the EMX-1 spacer.

FIGS. 15A and B show comparison of activity for VEGFA GX19 guide RNA variants at on- and off-target sites. A subset of guide RNA variants result in reduced activity at off-target sites, while retaining activity at on-target sites. FIG. 15B shows a comparison of on- and off-target activity for control sgRNA, GV-15 and GV19.

FIG. 16 shows the activity of guide RNA variants with either the first or second hairpin deleted. Boxes represent illustrative modifications.

FIG. 17 shows the activity of engineered guide RNA variants with altered nexus. Boxes represent illustrative modifications.

FIG. 18 shows the activity of engineered guide RNA variants with an increased loop in the nexus hairpin. Boxes represent illustrative modifications.

FIG. 19 shows biochemical activity of Cas9 and GX20 and NX19 engineered nucleic acid-targeting nucleic acids. The figure shows cleavage of double-stranded DNA amplified from human genomic DNA comprising an on-target protospacer and four off-target protospacers. GX20 engineered nucleic acid-targeting nucleic acids demonstrate a higher ratio of on-target activity to off-target activity compared with other engineered nucleic acid-targeting nucleic acids.

### DETAILED DESCRIPTION

### Definitions

As used herein, “affinity tag” can refer to either a peptide affinity tag or a nucleic acid affinity tag. Affinity tag generally refers to a protein or nucleic acid sequence that can be bound to a molecule (e.g., bound by a small molecule, protein, covalent bond). An affinity tag can be a non-native sequence. A peptide affinity tag can comprise a peptide. A peptide affinity tag can be one that is able to be part of a split system (e.g., two inactive peptide fragments can combine together in trans to form an active affinity tag). A nucleic acid affinity tag can comprise a nucleic acid. A nucleic acid affinity tag can be a sequence that can selectively bind to a known nucleic acid sequence (e.g. through hybridization). A nucleic acid affinity tag can be a sequence that can selectively bind to a protein. An affinity tag can be fused to a native protein. An affinity tag can be fused to a nucleotide sequence. Sometimes, one, two, or a plurality of affinity tags can be fused to a native protein or nucleotide sequence. An affinity tag can be introduced into a nucleic acid-targeting nucleic acid using methods of in vitro or in vivo transcription. Nucleic acid affinity tags can include, for example, a chemical tag, an RNA-binding protein binding sequence, a DNA-binding protein binding sequence, a sequence hybridizable to an affinity-tagged polynucleotide, a synthetic RNA aptamer, or a synthetic DNA aptamer. Examples of chemical nucleic acid affinity tags can include, but are not limited to, ribo-nucleotriphosphates containing biotin, fluorescent dyes, and digoxeginin. Examples of protein-binding nucleic acid affinity tags can include, but are not limited to, the MS2 binding sequence, the UlA binding sequence, stem-loop binding protein sequences, the boxB sequence, the eIF4A sequence, or any sequence recognized by an RNA binding protein. Examples of nucleic acid affinity-tagged oligonucleotides can include, but are not limited to, biotinylated oligonucleotides, 2,4-dinitrophenyl oligonucleotides, fluorescein oligonucleotides, and primary amine-conjugated oligonucleotides.

A nucleic acid affinity tag can be an RNA aptamer. Aptamers can include, aptamers that bind to theophylline, streptavidin, dextran B512, adenosine, guanosine, guanine/xanthine, 7-methyl-GTP, amino acid aptamers such as aptamers that bind to arginine, citrulline, valine, tryptophan, cyanocobalamine, N-methylmesoporphyrin IX, flavin, NAD, and antibiotic aptamers such as aptamers that bind to tobramycin, neomycin, lividomycin, kanamycin, streptomycin, viomycin, and chloramphenicol.

A nucleic acid affinity tag can comprise an RNA sequence that can be bound by a site-directed polypeptide. The site-directed polypeptide can be conditionally enzymatically inactive. The RNA sequence can comprise a sequence that can be bound by a member of Type I, Type II, and/or Type III CRISPR systems. The RNA sequence can be bound by a RAMP family member protein. The RNA sequence can be bound by a Cas6 family member protein (e.g., Csy4, Cas6). The RNA sequence can be bound by a Cas5 family member protein (e.g., Cas5). For example, Csy4 can bind to a specific RNA hairpin sequence with high affinity (Kd˜50 pM) and can cleave RNA at a site 3′ to the hairpin. The Cas5 or Cas6 family member protein can bind an RNA sequence that comprises at least about or at most about 30%, 40%, 50%, 60%, 70%, 75%, 80%, 85%, 90%, 95%, or 100% sequence identity and/or sequence similarity to the following nucleotide sequences: 5′-GUUCACUGCCGUAUAGGCAGCUAAGAAA-3′; 5′-GUUCACUGCCGUAUAGGCAGCUAAGAAA-3′ 5′-GUUGCAAGGGAUUGAGCCCCGUAAGGGGAUUGCGAC-3′; 5′-GUUGCAAACCUCGUUAGCCUCGUAGAGGAUUGAAAC-3′; 5′-GGAUCGAUACCCACCCCGAAGAAAAGGGGACGAGAAC-3′; 5′-GUCGUCAGACCCAAAACCCCGAGAGGGGACGGAAAC-3′; 5′-GAUAUAAACCUAAUUACCUCGAGAGGGGACGGAAAC-3′; 5′-CCCCAGUCACCUCGGGAGGGGACGGAAAC-3′; 5′-GUUCCAAUUAAUCUUAAACCCUAUUAGGGAUUGAAAC-3′. 5′-GUUGCAAGGGAUUGAGCCCCGUAAGGGGAUUGCGAC-3′; 5′-GUUGCAAACCUCGUUAGCCUCGUAGAGGAUUGAAAC-3′; 5′-GGAUCGAUACCCACCCCGAAGAAAAGGGGACGAGAAC-3′; 5′-GUCGUCAGACCCAAAACCCCGAGAGGGGACGGAAAC-3′; 5′-GAUAUAAACCUAAUUACCUCGAGAGGGGACGGAAAC-3′; 5′-CCCCAGUCACCUCGGGAGGGGACGGAAAC-3′; 5′-GUUCCAAUUAAUCUUAAACCCUAUUAGGGAUUGAAAC-3′, 5′-GUCGCCCCCCACGCGGGGGCGUGGAUUGAAAC-3′; 5′-CCAGCCGCCUUCGGGCGGCUGUGUGUUGAAAC-3′; 5′-GUCGCACUCUACAUGAGUGCGUGGAUUGAAAU-3′; 5′-UGUCGCACCUUAUAUAGGUGCGUGGAUUGAAAU-3′; and 5′-GUCGCGCCCCGCAUGGGGCGCGUGGAUUGAAA-3′,

A nucleic acid affinity tag can comprise a DNA sequence that can be bound by a site-directed polypeptide. The site-directed polypeptide can be conditionally enzymatically inactive. The DNA sequence can comprise a sequence that can be bound by a member of the Type I, Type II and/or Type III CRISPR system. The DNA sequence can be bound by an Argonaut protein. The DNA sequence can be bound by a protein containing a zinc finger domain, a TALE domain, or any other DNA-binding domain.

A nucleic acid affinity tag can comprise a ribozyme sequence. Suitable ribozymes can include peptidyl transferase 23S rRNA, RNaseP, Group I introns, Group II introns, GIR1 branching ribozyme, Leadzyme, hairpin ribozymes, hammerhead ribozymes, HDV ribozymes, CPEB3 ribozymes, VS ribozymes, glmS ribozyme, CoTC ribozyme, and synthetic ribozymes.

Peptide affinity tags can comprise tags that can be used for tracking or purification (e.g., a fluorescent protein, green fluorescent protein (GFP), YFP, RFP, CFP, mCherry, tdTomato, a his tag, (e.g., a 6XHis tag), a hemagglutinin (HA) tag, a FLAG tag, a Myc tag, a GST tag, a MBP tag, and chitin binding protein tag, a calmodulin tag, a V5 tag, a streptavidin binding tag, and the like).

Both nucleic acid and peptide affinity tags can comprise small molecule tags such as biotin, or digitoxin, fluorescent label tags, such as for example, fluoroscein, rhodamin, ALEXA FLUOR dyes, Cyanine3 dye, Cyanine5 dye.

Nucleic acid affinity tags can be located 5′ to a nucleic acid (e.g., a nucleic acid-targeting nucleic acid, sgRNA, guide RNA variant). Nucleic acid affinity tags can be located 3′ to a nucleic acid. Nucleic acid affinity tags can be located 5′ and 3′ to a nucleic acid. Nucleic acid affinity tags can be located within a nucleic acid. Peptide affinity tags can be located N-terminal to a polypeptide sequence. Peptide affinity tags can be located C-terminal to a polypeptide sequence. Peptide affinity tags can be located N-terminal and C-terminal to a polypeptide sequence. A plurality of affinity tags can be fused to a nucleic acid and/or a polypeptide sequence.

As used herein, “Cas9” can generally refer to a polypeptide with at least about 5%, 10%, 20%, 30%, 40%, 50%, 60%, 70%, 80%, 90%, 100% sequence identity and/or sequence similarity to a wild type exemplary Cas9 polypeptide, for example, Cas9 from S. pyogenes (SEQ ID NO: 8) or to any of the amino acid sequences set forth in SEQ ID NOs: 1-256 and 795-1346. Cas9 can refer to a polypeptide with at most about 5%, 10%, 20%, 30%, 40%, 50%, 60%, 70%, 80%, 90%, 100% sequence identity and/or sequence similarity to a wild type exemplary Cas9 polypeptide, for example, Cas9 from S. pyogenes (SEQ ID NO: 8) or to any of the amino acid sequences set forth in SEQ ID NOs: 1-256 and 795-1346. Cas9 can refer to the wild-type or a modified form of the Cas9 protein that can comprise an amino acid change such as a deletion, insertion, substitution, variant, mutation, fusion, chimera, or any combination thereof.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp) a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), and a cell from a mammal (e.g., a pig, a cow, a goat, a sheep, a rodent, a rat, a mouse, a non-human primate, and a human). Sometimes a cell is not originating from a natural organism (e.g. a cell can be a synthetically made, sometimes termed an artificial cell).

A cell can be in vitro. A cell can be in vivo. A cell can be an isolated cell. A cell can be a cell inside of an organism. A cell can be an organism. A cell can be a cell in a cell culture. A cell can be one of a collection of cells. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a mammalian cell or derived from a mammalian cell. A cell can be a rodent cell or derived from a rodent cell. A cell can be a human cell or derived from a human cell. A cell can be a microbe cell or derived from a microbe cell. A cell can be a fungi cell or derived from a fungi cell.

A cell can be a stem cell or progenitor cell. Cells can include stem cells (e.g., adult stem cells, embryonic stem cells, iPS cells) and progenitor cells (e.g., cardiac progenitor cells, neural progenitor cells, etc.). Cells can include mammalian stem cells and progenitor cells, including rodent stem cells, rodent progenitor cells, human stem cells, human progenitor cells, etc. Clonal cells can comprise the progeny of a cell. A cell can comprise a target nucleic acid. A cell can be in a living organism. A cell can be a genetically modified cell. A cell can be a host cell.

A cell can be a totipotent stem cell, however, in some embodiments of this disclosure, the term “cell” may be used but may not refer to a totipotent stem cell. A cell can be a plant cell, but in some embodiments of this disclosure, the term “cell” may be used but may not refer to a plant cell. A cell can be a pluripotent cell. For example, a cell can be a pluripotent hematopoietic cell that can differentiate into other cells in the hematopoietic cell lineage but may not be able to differentiate into any other non-hematopoetic cell. A cell may be able to develop into a whole organism. A cell may or may not be able to develop into a whole organism. A cell may be a whole organism.

A cell can be a primary cell. For example, cultures of primary cells can be passaged 0 times, 1 time, 2 times, 4 times, 5 times, 10 times, 15 times or more. Cells can be unicellular organisms. Cells can be grown in culture.

A cell can be a diseased cell. A diseased cell can have altered metabolic, gene expression, and/or morphologic features. A diseased cell can be a cancer cell, a diabetic cell, and an apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g., normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

As used herein, “crRNA” can generally refer to a nucleic acid with at least about 5%, 10%, 20%, 30%, 40%, 50%, 60%, 70%, 80%, 90%, 100% sequence identity and/or sequence similarity to a wild type exemplary crRNA (e.g., a crRNA from S. pyogenes (e.g., SEQ ID NO: 569), SEQ ID NOs: 563-679). crRNA can generally refer to a nucleic acid with at most about 5%, 10%, 20%, 30%, 40%, 50%, 60%, 70%, 80%, 90%, 100% sequence identity and/or sequence similarity to a wild type exemplary crRNA (e.g., a crRNA from S. pyogenes). crRNA can refer to a modified form of a crRNA that can comprise an nucleotide change such as a deletion, insertion, or substitution, variant, mutation, or chimera. A crRNA can be a nucleic acid having at least about 60% identical to a wild type exemplary crRNA (e.g., a crRNA from S. pyogenes) sequence over a stretch of at least 6 contiguous nucleotides. For example, a crRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical, to a wild type exemplary crRNA sequence (e.g., a crRNA from S. pyogenes) over a stretch of at least 6 contiguous nucleotides.

As used herein, “CRISPR repeat” or “CRISPR repeat sequence” can refer to a minimum CRISPR repeat sequence.

As used herein, “endoribonuclease” can generally refer to a polypeptide that can cleave RNA. In some embodiments, an endoribonuclease can be a site-directed polypeptide. An endoribonuclease may be a member of a CRISPR system (e.g., Type I, Type II, Type III). Endoribonuclease can refer to a Repeat Associated Mysterious Protein (RAMP) superfamily of proteins (e.g., Cas6, Cas6, Cas5 families). Endoribonucleases can also include RNase A, RNase H, RNase I, RNase III family members (e.g., Drosha, Dicer, RNase N), RNase L, RNase P, RNase PhyM, RNase T1, RNase T2, RNase U2, RNase V1, RNase V. An endoribonuclease can refer to a conditionally enzymatically inactive endoribonuclease. An endoribonuclease can refer to a catalytically inactive endoribonuclease.

As used herein, “donor polynucleotide” can refer to a nucleic acid that can be integrated into a site during genome engineering or target nucleic acid engineering.

As used herein, “fixative” or “cross-linker” can generally refer to an agent that can fix or cross-link cells. Fixed or cross-linking cells can stabilize protein-nucleic acid complexes in the cell. Suitable fixatives and cross-linkers can include, formaldehyde, glutaraldehyde, ethanol-based fixatives, methanol-based fixatives, acetone, acetic acid, osmium tetraoxide, potassium dichromate, chromic acid, potassium permanganate, mercurials, picrates, formalin, paraformaldehyde, amine-reactive NHS-ester crosslinkers such as bis[sulfosuccinimidyl] suberate (BS3), 3,3″-dithiobis[sulfosuccinimidylpropionate] (DTSSP), ethylene glycol bis[sulfosuccinimidylsuccinate (sulfo-EGS), disuccinimidyl glutarate (DSG), dithiobis[succinimidyl propionate] (DSP), disuccinimidyl suberate (DSS), ethylene glycol bis[succinimidylsuccinate] (EGS), NHS-ester/diazirine crosslinkers such as NHS-diazirine, NHS-LC-diazirine, NHS-SS-diazirine, sulfo-NHS-diazirine, sulfo-NHS-LC-diazirine, and sulfo-NHS-SS-diazirine.

As used herein, “fusion” can refer to a protein and/or nucleic acid comprising one or more non-native sequences (e.g., moieties). A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as ALEXA FLUOR dyes, Cyanine3 dye, Cyanine5 dye. The fusion can provide for increased or decreased stability.

In some embodiments, a fusion can comprise a detectable label, including a moiety that can provide a detectable signal. Suitable detectable labels and/or moieties that can provide a detectable signal can include, but are not limited to, an enzyme, a radioisotope, a member of a specific binding pair; a fluorophore; a fluorescent protein; a quantum dot; and the like.

A fusion can comprise a member of a FRET pair. FRET pairs (donor/acceptor) suitable for use can include, but are not limited to, EDANS/fluorescein, IAEDANS/fluorescein, fluorescein/tetramethylrhodamine, fluorescein/Cy 5, IEDANS/DABCYL, fluorescein/QSY-7, fluorescein/LC Red 640, fluorescein/Cy 5.5 and fluorescein/LC Red 705.

A fluorophore/quantum dot donor/acceptor pair can be used as a fusion. Suitable fluorophores (“fluorescent label”) can include any molecule that may be detected via its inherent fluorescent properties, which can include fluorescence detectable upon excitation. Suitable fluorescent labels can include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green.

A fusion can comprise an enzyme. Suitable enzymes can include, but are not limited to, horse radish peroxidase, luciferase, beta-galactosidase, and the like.

A fusion can comprise a fluorescent protein. Suitable fluorescent proteins can include, but are not limited to, a green fluorescent protein (GFP), (e.g., a GFP from Aequoria victoria, fluorescent proteins from Anguilla japonica, or a mutant or derivative thereof), a red fluorescent protein, a yellow fluorescent protein, any of a variety of fluorescent and colored proteins.

A fusion can comprise a nanoparticle. Suitable nanoparticles can include fluorescent or luminescent nanoparticles, and magnetic nanoparticles. Any optical or magnetic property or characteristic of the nanoparticle(s) can be detected.

A fusion can comprise quantum dots (QDs). QDs can be rendered water-soluble by applying coating layers comprising a variety of different materials. For example, QDs can be solubilized using amphiphilic polymers. Exemplary polymers that have been employed can include octylamine-modified low molecular weight polyacrylic acid, polyethylene-glycol (PEG)-derivatized phospholipids, polyanhydrides, block copolymers, etc. QDs can be conjugated to a polypeptide via any of a number of different functional groups or linking agents that can be directly or indirectly linked to a coating layer. QDs with a wide variety of absorption and emission spectra are commercially available, e.g., from Quantum Dot Corp. (Hayward Calif.; now owned by Invitrogen) or from Evident Technologies (Troy, N.Y.). For example, QDs having peak emission wavelengths of approximately 525, 535, 545, 565, 585, 605, 655, 705, and 800 nm are available. Thus the QDs can have a range of different colors across the visible portion of the spectrum and in some cases even beyond.

Suitable radioisotopes can include, but are not limited to ¹⁴C, ³H, ³²P, ³³P, ³⁵S, and ¹²⁵I.

As used herein, “genetically modified cell” can generally refer to a cell that has been genetically modified. Some non-limiting examples of genetic modifications can include: insertions, deletions, inversions, translocations, gene fusions, or changing one or more nucleotides. A genetically modified cell can comprise a target nucleic acid with an introduced double strand break (e.g., DNA break). A genetically modified cell can comprise an exogenously introduced nucleic acid (e.g., a vector). A genetically modified cell can comprise an exogenously introduced polypeptide of the disclosure and/or nucleic acid of the disclosure. A genetically modified cell can comprise a donor polynucleotide. A genetically modified cell can comprise an exogenous nucleic acid integrated into the genome of the genetically modified cell. A genetically modified cell can comprise a deletion of DNA. A genetically modified cell can also refer to a cell with modified mitochondrial or chloroplast DNA.A genetically modified cell can comprise any modification described herein.

As used herein, “genome engineering” can refer to a process of modifying a target nucleic acid. Genome engineering can refer to the integration of non-native nucleic acid into native nucleic acid. Genome engineering can refer to the targeting of a site-directed polypeptide and a nucleic acid-targeting nucleic acid to a target nucleic acid, without an integration or a deletion of the target nucleic acid. Genome engineering can refer to the cleavage of a target nucleic acid, and the rejoining of the target nucleic acid without an integration of an exogenous sequence in the target nucleic acid, or a deletion in the target nucleic acid. The native nucleic acid can comprise a gene. The non-native nucleic acid can comprise a donor polynucleotide. In the methods of the disclosure, site-directed polypeptides (e.g., Cas9) can introduce double-stranded breaks in nucleic acid, (e.g. genomic DNA). The double-stranded break can stimulate a cell's endogenous DNA-repair pathways (e.g. homologous recombination (HR) and/or non-homologous end joining (NHEJ), or A-NHEJ (alternative non-homologous end-joining)). Mutations, deletions, alterations, and integrations of foreign, exogenous, and/or alternative nucleic acid can be introduced into the site of the double-stranded DNA break.

As used herein, the term “isolated” can refer to a nucleic acid or polypeptide that, by the hand of a human, exists apart from its native environment and is therefore not a product of nature. Isolated can mean substantially pure. An isolated nucleic acid or polypeptide can exist in a purified form and/or can exist in a non-native environment such as, for example, in a transgenic cell.

As used herein, “non-native” can refer to a nucleic acid or polypeptide sequence that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide. A non-native sequence can refer to a 3′ hybridizing extension sequence.

As used herein, a “nucleic acid” can generally refer to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g. altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or flurescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine.

As used herein, a “nucleic acid sample” can generally refer to a sample from a biological entity. A nucleic acid sample can comprise nucleic acid. The nucleic acid from the nucleic acid sample can be purified and/or enriched. The nucleic acid sample may show the nature of the whole. Nucleic acid samples can come from various sources. Nucleic acid samples can come from one or more individuals. One or more nucleic acid samples can come from the same individual. One non-limiting example would be if one sample came from an individual's blood and a second sample came from an individual's tumor biopsy. Examples of nucleic acid samples can include but are not limited to, blood, serum, plasma, nasal swab or nasopharyngeal wash, saliva, urine, gastric fluid, spinal fluid, tears, stool, mucus, sweat, earwax, oil, glandular secretion, cerebral spinal fluid, tissue, semen, vaginal fluid, interstitial fluids, including interstitial fluids derived from tumor tissue, ocular fluids, spinal fluid, throat swab, cheek swab, breath, hair, finger nails, skin, biopsy, placental fluid, amniotic fluid, cord blood, emphatic fluids, cavity fluids, sputum, pus, micropiota, meconium, breast milk, buccal samples, nasopharyngeal wash, other excretions, or any combination thereof. Nucleic acid samples can originate from tissues. Examples of tissue samples may include but are not limited to, connective tissue, muscle tissue, nervous tissue, epithelial tissue, cartilage, cancerous or tumor sample, bone marrow, or bone. The nucleic acid sample may be provided from a human or animal. The nucleic acid sample may be provided from a mammal, vertebrate, such as murines, simians, humans, farm animals, sport animals, or pets. The nucleic acid sample may be collected from a living or dead subject. The nucleic acid sample may be collected fresh from a subject or may have undergone some form of pre-processing, storage, or transport.

A nucleic acid sample can comprise a target nucleic acid. A nucleic acid sample can originate from cell lysate. The cell lysate can originate from a cell.

As used herein, “nucleic acid-targeting nucleic acid” can refer to a nucleic acid that can hybridize to another nucleic acid. A nucleic acid-targeting nucleic acid can be RNA. A nucleic acid-targeting nucleic acid can be DNA. A nucleic acid-targeting nucleic acid can comprise DNA and RNA residues, for example, a DNA/RNA hybrid. The nucleic acid-targeting nucleic acid can be programmed to bind to a sequence of nucleic acid site-specifically. The nucleic acid to be targeted, or the target nucleic acid, can comprise nucleotides. The nucleic acid-targeting nucleic acid can comprise nucleotides. A portion of the target nucleic acid can be complementary to a portion of the nucleic acid-targeting nucleic acid. A nucleic acid-targeting nucleic acid can comprise a polynucleotide chain and can be called a “single guide nucleic acid” (i.e. a “single guide nucleic acid-targeting nucleic acid”). A nucleic acid-targeting nucleic acid can comprise two polynucleotide chains and can be called a “double guide nucleic acid” (i.e. a “double guide nucleic acid-targeting nucleic acid”). A nucleic acid-targeting nucleic acid can be a single guide RNA (sgRNA). A nucleic acid-targeting nucleic acid can be a guide RNA variant. If not otherwise specified, the term “nucleic acid-targeting nucleic acid” can be inclusive, referring to both single guide nucleic acids and double guide nucleic acids.

A nucleic acid-targeting nucleic acid can comprise a segment that can be referred to as a “nucleic acid-targeting segment” or a “nucleic acid-targeting sequence,” (eg., “a spacer”). A nucleic acid-targeting nucleic acid can comprise a segment that can be referred to as a “protein binding segment” or “protein binding sequence.”

A nucleic acid-targeting nucleic acid can comprise one or more modifications (e.g., a base modification, a backbone modification), to provide the nucleic acid with a new or enhanced feature (e.g., improved stability). A nucleic acid-targeting nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Purines can be adenine and guanine Pyrimidines can be cytosine, uracil, and thymine. Nucleotides can be nucleosides that further include a phosphate group covalently linked to the sugar portion of the nucleoside (e.g., nucleoside di-phosphate, nucleoside tri-phosphate). For those nucleosides that include a pentofuranosyl sugar, the phosphate group can be linked to the 2′, the 3′, or the 5′ hydroxyl moiety of the sugar. In forming nucleic acid-targeting nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acid-targeting nucleic acids, the phosphate groups can commonly be referred to as forming the internucleoside backbone of the nucleic acid-targeting nucleic acid. The linkage or backbone of the nucleic acid-targeting nucleic acid can be a 3′ to 5′ phosphodiester linkage. As used herein, the purine and pyrimidine bases of adenine, guanine, cytosine, uracil, and thymine, can refer to the nucleoside form, the nucleotide form, the nucleoside di-phosphate form and/or, the nucleoside tri-phosphate form of the base.

A nucleic acid-targeting nucleic acid can comprise a modified backbone and/or modified internucleoside linkages. Modified backbones can include those that retain a phosphorus atom in the backbone and those that do not have a phosphorus atom in the backbone.

Suitable modified nucleic acid-targeting nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable nucleic acid-targeting nucleic acids having inverted polarity can comprise a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

A nucleic acid-targeting nucleic acid can comprise one or more phosphorothioate and/or heteroatom internucleoside linkages, in particular —CH₂—NH—O—CH₂—, —CH₂—N(CH₃)—O—CH₂— (i.e. a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—).

A nucleic acid-targeting nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid-targeting nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid-targeting nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza-nitrogen atoms of the amide portion of the backbone.

A nucleic acid-targeting nucleic acid can comprise linked morpholino units (i.e. morpholino nucleic acid) having heterocyclic bases attached to the morpholino ring Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acid-targeting nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH2-), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid-targeting nucleic acid can comprise one or more substituted sugar moieties. Suitable polynucleotides can comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH2)nO) mCH₃, O(CH₂)ₙOCH₃, O(CH₂)ₙNH2, O(CH₂)ₙCH₃, O(CH₂)ₙONH₂, and O(CH₂)ₙON((CH₂)ₙCH₃)₂, where n and m are from 1 to about 10. A sugar substituent group can be selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an nucleic acid-targeting nucleic acid, or a group for improving the pharmacodynamic properties of an nucleic acid-targeting nucleic acid, and other substituents having similar properties. A suitable modification can include 2′-methoxyethoxy (2′-O—CH₂ CH₂OCH₃, also known as 2′-O-(2-methoxyethyl) or 2′-MOE i.e., an alkoxyalkoxy group). A further suitable modification can include 2′-dimethylaminooxyethoxy, (i.e., a O(CH₂)₂ON(CH₃)₂ group, also known as 2′-DMAOE), and 2′-dimethylaminoethoxyethoxy (also known as 2′-O-dimethyl-amino-ethoxy-ethyl or 2′-DMAEOE), i.e., 2′-O—CH2-O—CH₂—N(CH₃)₂.

Other suitable sugar substituent groups can include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds may also have sugar mimetics such as cyclobutyl moieties in place of the pentofuranosyl sugar.

A nucleic acid-targeting nucleic acid may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (Hpyrido(3′,2′:4,5)pyrrolo(2,3-d)pyrimidin-2-one).

Heterocyclic base moieties can include those in which the purine or pyrimidine base is replaced with other heterocycles, for example 7-deaza-adenine, 7-deazaguanosine, 2-aminopyridine and 2-pyridone. Nucleobases can be useful for increasing the binding affinity of a polynucleotide compound. These can include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

A modification of a nucleic acid-targeting nucleic acid can comprise chemically linking to the nucleic acid-targeting nucleic acid one or more moieties or conjugates that can enhance the activity, cellular distribution or cellular uptake of the nucleic acid-targeting nucleic acid. These moieties or conjugates can include conjugate groups covalently bound to functional groups such as primary or secondary hydroxyl groups. Conjugate groups can include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups can include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties can include but are not limited to lipid moieties such as a cholesterol moiety, cholic acid a thioether, (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety.

A modification may include a “Protein Transduction Domain” or PTD (i.e. a cell penetrating peptide (CPP)). The PTD can refer to a polypeptide, polynucleotide, carbohydrate, or organic or inorganic compound that facilitates traversing a lipid bilayer, micelle, cell membrane, organelle membrane, or vesicle membrane. A PTD can be attached to another molecule, which can range from a small polar molecule to a large macromolecule and/or a nanoparticle, and can facilitate the molecule traversing a membrane, for example going from extracellular space to intracellular space, or cytosol to within an organelle. A PTD can be covalently linked to the amino terminus of a polypeptide. A PTD can be covalently linked to the carboxyl terminus of a polypeptide. A PTD can be covalently linked to a nucleic acid. Exemplary PTDs can include, but are not limited to, a minimal peptide protein transduction domain; a polyarginine sequence comprising a number of arginines sufficient to direct entry into a cell (e.g., 3, 4, 5, 6, 7, 8, 9, 10, or 10-50 arginines), a VP22 domain, a Drosophila Antennapedia protein transduction domain, a truncated human calcitonin peptide, polylysine, and transportan, arginine homopolymer of from 3 arginine residues to 50 arginine residues. The PTD can be an activatable CPP (ACPP). ACPPs can comprise a polycationic CPP (e.g., Arg9 or “R9”) connected via a cleavable linker to a matching polyanion (e.g., Glu9 or “E9”), which can reduce the net charge to nearly zero and thereby inhibits adhesion and uptake into cells. Upon cleavage of the linker, the polyanion can be released, locally unmasking the polyarginine and its inherent adhesiveness, thus “activating” the ACPP to traverse the membrane.

“Nucleotide” can generally refer to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo)benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

As used herein, “Nexus” can refer to a region in a nucleic acid-targeting nucleic acid. The nexus confers the binding of a sgRNA or a tracrRNA to its cognate Cas9 protein and confers an apoenzyme to haloenzyme conformational transition.

As used here, “purified” can refer to a molecule (e.g., site-directed polypeptide, nucleic acid-targeting nucleic acid) that comprises at least 50, 60, 70, 80, 90, 95, 96, 97, 98, 99, or 100% of the composition. For example, a sample that comprises 10% of a site-directed polypeptide, but after a purification step comprises 60% of the site-directed polypeptide, then the sample can be said to be purified. A purified sample can refer to an enriched sample, or a sample that has undergone methods to remove particles other than the particle of interest.

As used herein, “recombinant” can refer to sequence that originates from a source foreign to the particular host (e.g., cell) or, if from the same source, is modified from its original form. A recombinant nucleic acid in a cell can include a nucleic acid that is endogenous to the particular cell but has been modified through, for example, the use of site-directed mutagenesis. The term can include non-naturally occurring multiple copies of a naturally occurring DNA sequence. Thus, the term can refer to a nucleic acid that is foreign or heterologous to the cell, or homologous to the cell but in a position or form within the cell in which the nucleic acid is not ordinarily found. Similarly, when used in the context of a polypeptide or amino acid sequence, an exogenous polypeptide or amino acid sequence can be a polypeptide or amino acid sequence that originates from a source foreign to the particular cell or, if from the same source, is modified from its original form.

As used herein, “non-natural nucleic acid-targeting nucleic acid” can refer to a nucleic acid-targeting nucleic acid that has one or more non-naturally occurring regions. A non-naturally occurring nucleic acid-targeting nucleic acids can be selected over naturally occurring forms because of desirable properties such as, for example, reduced off-target effects, enhanced cellular uptake, enhanced affinity for nucleic acid targets, and increased stability in the presence of nucleases. A non-natural nucleic acid-targeting nucleic acid can be a designed nucleic acid-targeting nucleic acid. A non-natural nucleic acid-targeting nucleic acid can be an engineered nucleic acid-targeting nucleic acid. A non-natural nucleic acid-targeting nucleic acid can be an isolated and/or recombinant nucleic acid-targeting nucleic acid.

As used herein, “control nucleic acid-targeting nucleic acid” can refer to a nucleic acid-targeting nucleic acid that has not been modified. A control nucleic acid-targeting nucleic acid can be a naturally occurring nucleic acid-targeting nucleic acid. A control nucleic acid-targeting nucleic acid can be a wild-type nucleic acid-targeting nucleic acid. A control nucleic acid-targeting nucleic acid can be a non-engineered form of a nucleic acid-targeting nucleic acid.

As used herein, “site-directed polypeptides” can generally refer to nucleases, site-directed nucleases, endoribonucleases, conditionally enzymatically inactive endoribonucleases, Argonaute, and nucleic acid-binding proteins. A site-directed polypeptide or protein can include nucleases such as homing endonucleases such as PI-TliII, H-DreI, I-DmoI and I-CreI, I-SceI, LAGLIDADG family nucleases, meganucleases, GIY-YIG family nucleases, His-Cys box family nucleases, Vsr-like nucleases, endoribonucleases, exoribonucleases, endonucleases, and exonucleases. A site-directed polypeptide can refer to a Cas gene member of the Type I, Type II, Type III, and/or Type U CRISPR/Cas systems. A site-directed polypeptide can refer to a member of the Repeat Associated Mysterious Protein (RAMP) superfamily (e.g., Cas5, Cas6 subfamilies). A site-directed polypeptide can refer to an Argonaute protein.

A site-directed polypeptide can be a type of protein. A site-directed polypeptide can refer to a nuclease. A site-directed polypeptide can refer to an endoribonuclease. A site-directed polypeptide can refer to any modified (e.g., shortened, mutated, lengthened) polypeptide sequence or homologue of the site-directed polypeptide. A site-directed polypeptide can be codon optimized. A site-directed polypeptide can be a codon-optimized homologue of a site-directed polypeptide. A site-directed polypeptide can be enzymatically inactive, partially active, constitutively active, fully active, inducible active and/or more active, (e.g. more than the wild type homologue of the protein or polypeptide). A site-directed polypeptide can be Cas9. A site-directed polypeptide can be Csy4. A site-directed polypeptide can be Cas5 or a Cas5 family member. A site-directed polypeptide can be Cas6 or a Cas6 family member. SEQ ID NOs: 1-256 and 795-1346 provide a non-limiting and non-exhaustive list of naturally occurring Cas9/Csn1 endonucleases that can be used as site-directed polypeptides in the wild-type, variant, or mutated form.

In some instances, the site-directed polypeptide (e.g., variant, mutated, enzymatically inactive and/or conditionally enzymatically inactive site-directed polypeptide) can target nucleic acid. The site-directed polypeptide (e.g., variant, mutated, enzymatically inactive and/or conditionally enzymatically inactive endoribonuclease) can target RNA. Endoribonucleases that can target RNA can include members of other CRISPR subfamilies such as Cas6 and Cas5.

As used herein, the term “specific” can refer to interaction of two molecules where one of the molecules through, for example chemical or physical means, specifically binds to the second molecule. Exemplary specific binding interactions can refer to antigen-antibody binding, avidin-biotin binding, carbohydrates and lectins, complementary nucleic acid sequences (e.g., hybridizing), complementary peptide sequences including those formed by recombinant methods, effector and receptor molecules, enzyme cofactors and enzymes, enzyme inhibitors and enzymes, and the like. “Non-specific” can refer to an interaction between two molecules that is not specific.

As used herein, “solid support” can generally refer to any insoluble, or partially soluble material. A solid support can refer to a test strip, a multi-well dish, and the like. The solid support can comprise a variety of substances (e.g., glass, polystyrene, polyvinyl chloride, polypropylene, polyethylene, polycarbonate, dextran, nylon, amylose, natural and modified celluloses, polyacrylamides, agaroses, and magnetite) and can be provided in a variety of forms, including agarose beads, polystyrene beads, latex beads, magnetic beads, colloid metal particles, glass and/or silicon chips and surfaces, nitrocellulose strips, nylon membranes, sheets, wells of reaction trays (e.g., multi-well plates), plastic tubes, etc. A solid support can be solid, semisolid, a bead, or a surface. The support can mobile in a solution or can be immobile. A solid support can be used to capture a polypeptide. A solid support can comprise a capture agent.

As used herein, “target nucleic acid” can generally refer to a nucleic acid to be used in the methods of the disclosure. A target nucleic acid can refer to a chromosomal sequence or an extrachromosomal sequence, (e.g. an episomal sequence, a minicircle sequence, a mitochondrial sequence, a chloroplast sequence, etc.). A target nucleic acid can be DNA. A target nucleic acid can be RNA. A target nucleic acid can herein be used interchangeably with “polynucleotide”, “nucleotide sequence”, and/or “target polynucleotide”. A target nucleic acid can be a nucleic acid sequence that may not be related to any other sequence in a nucleic acid sample by a single nucleotide substitution. A target nucleic acid can be a nucleic acid sequence that may not be related to any other sequence in a nucleic acid sample by a 2, 3, 4, 5, 6, 7, 8, 9, or 10 nucleotide substitutions. In some embodiments, the substitution cannot occur within 5, 10, 15, 20, 25, 30, or 35 nucleotides of the 5′ end of a target nucleic acid. In some embodiments, the substitution cannot occur within 5, 10, 15, 20, 25, 30, 35 nucleotides of the 3′ end of a target nucleic acid.

As used herein, “tracrRNA” can generally refer to a nucleic acid with at least about 5%, 10%, 20%, 30%, 40%, 50%, 60%, 70%, 80%, 90%, 100% sequence identity and/or sequence similarity to a wild type exemplary tracrRNA sequence (e.g., a tracrRNA from S. pyogenes (SEQ ID NO:433), SEQ ID NOs: 431-562). tracrRNA can refer to a nucleic acid with at most about 5%, 10%, 20%, 30%, 40%, 50%, 60%, 70%, 80%, 90%, 100% sequence identity and/or sequence similarity to a wild type exemplary tracrRNA sequence (e.g., a tracrRNA from S. pyogenes). tracrRNA can refer to a modified form of a tracrRNA that can comprise an nucleotide change such as a deletion, insertion, or substitution, variant, mutation, or chimera. A tracrRNA can refer to a nucleic acid that can be at least about 60% identical to a wild type exemplary tracrRNA (e.g., a tracrRNA from S. pyogenes) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical, to a wild type exemplary tracrRNA (e.g., a tracrRNA from S. pyogenes) sequence over a stretch of at least 6 contiguous nucleotides. A tracrRNA can refer to a mid-tracrRNA. A tracrRNA can refer to a minimum tracrRNA sequence.

### General Overview

The disclosure provides compositions and methods for increasing the targeting specificity of a complex comprising a nucleic acid-targeting nucleic acid and a site-directed polypeptide. A nucleic acid-targeting nucleic acid can be engineered at the 5′ end to comprise 1, 2, 3, or more additional nucleotides. In some instances, a nucleic acid-targeting nucleic acid is engineered to consist of 1 additional nucleotide. A nucleic acid-targeting nucleic acid can be engineered at the 5′ end to delete 1, 2, or 3 nucleotides. The location of 5′ engineering can be directly adjacent to the spacer. In other words, the 5′ spacer extension can be 1, 2, or 3 additional nucleotides. The 5′ engineered nucleic acid-targeting nucleic acid can retain activity at a target nucleic acid site, while decreasing off-target binding.

The disclosure provides for compositions and methods for altering the efficacy of a nucleic acid-targeting nucleic acid. A nucleic acid-targeting nucleic acid can be engineered at the 3′ end to delete one or two hairpins of the 3′ tracrRNA extension sequence (also known as, the hairpin region). The 3′ engineered nucleic acid-targeting nucleic acid can be chemically synthesized.

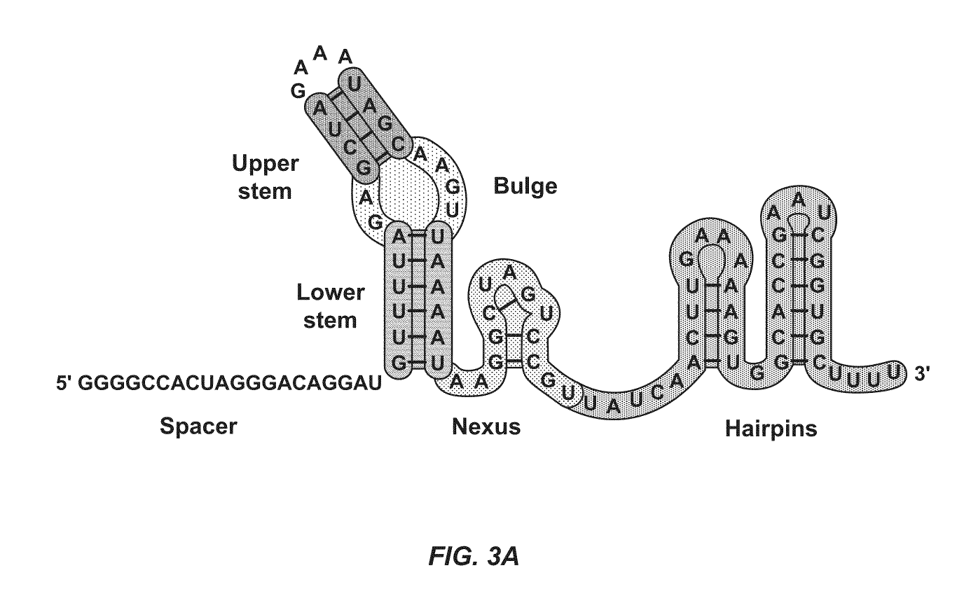

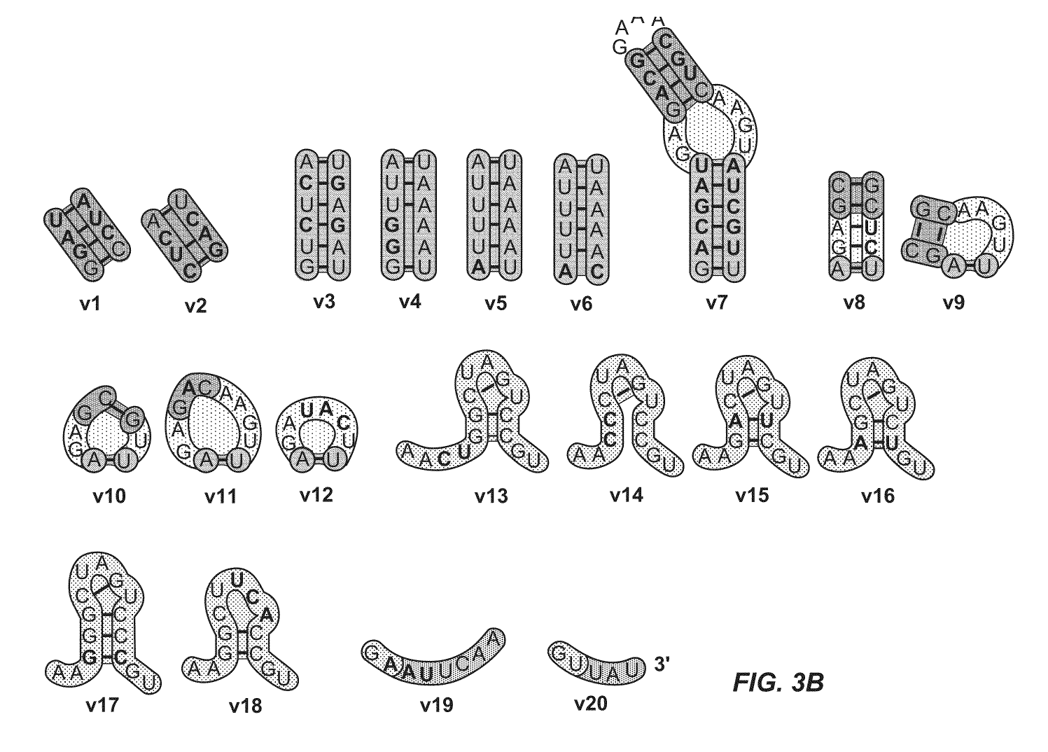

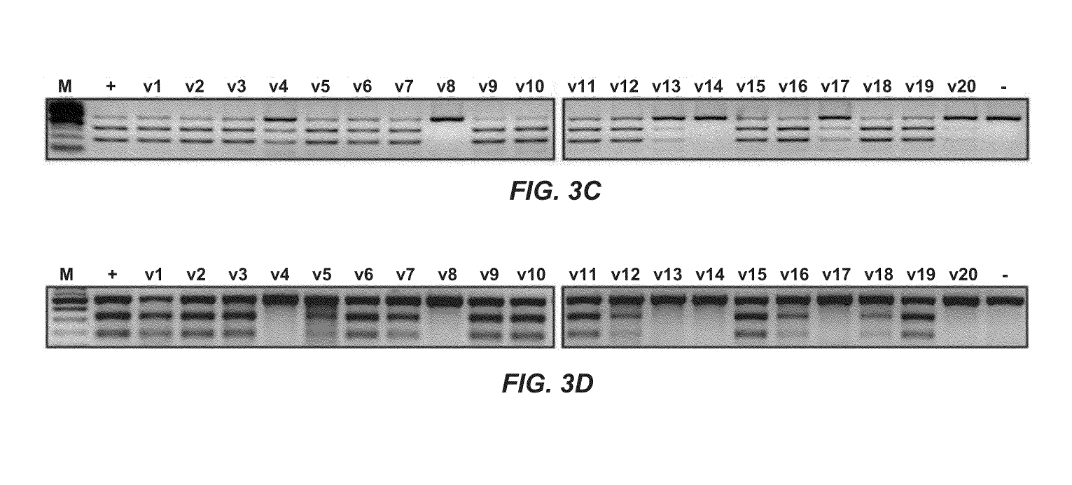

The disclosure provides for compositions and methods for generating a library of backbone variants of a nucleic acid-targeting nucleic acid (e.g., guide RNA variants). The variants in the library can be generated for any suitable region or sequence of the nucleic acid-targeting nucleic acid (FIGS. 3A and B), for example, in the 5′ spacer extension, spacer, lower stem, upper stem, bulge, nexus, loop, hairpin regions, or any combination thereof. The variants can comprise any suitable modifications of the residues, for example, deletion, insertion, substitution, variant, mutation, fusion, chimera, or any combination thereof. Modifications to nucleotides can include synthetic nucleotides, additional nucleotides, capped nucleotides, deoxyribonucleotides, or any combination thereof.

The variants in the library can be screened for characteristics such as binding efficacy, cleavage efficacy, and homologous recombination efficacy.

CRISPR Systems

A CRISPR (Clustered Regularly Interspaced Short Palindromic Repeats) can be a genomic locus found in the genomes of many prokaryotes (e.g., bacteria and archaea). CRISPR loci can provide resistance to foreign invaders (e.g., virus, phage) in prokaryotes. In this way, the CRISPR system can be thought to function as a type of immune system to help defend prokaryotes against foreign invaders. There can be three stages of CRISPR locus function: integration of new sequences into the locus, biogenesis of CRISPR RNA (crRNA), and silencing of foreign invader nucleic acid. There can be four types of CRISPR systems (e.g., Type I, Type II, Type III, TypeU).

A CRISPR locus can include a number of short repeating sequences referred to as “repeats.” Repeats can form hairpin structures and/or repeats can be unstructured single-stranded sequences. The repeats can occur in clusters. Repeats sequences can frequently diverge between species. Repeats can be regularly interspaced with unique intervening sequences referred to as “spacers,” resulting in a repeat-spacer-repeat locus architecture. Spacers can be identical to or have high homology with known foreign invader sequences. A spacer-repeat unit can encode a CRISPR RNA (crRNA). A crRNA can refer to the mature form of the spacer-repeat unit. A spacer can comprise a “seed” sequence that can be involved in targeting a target nucleic acid (e.g., possibly as a surveillance mechanism against foreign nucleic acid). A seed sequence can be located at the 5′ or 3′ end of the crRNA.

A CRISPR locus can comprise polynucleotide sequences encoding for CRISPR Associated Genes (Cas) genes. Cas genes can be involved in the biogenesis and/or the interference stages of crRNA function. Cas genes can display extreme sequence (e.g., primary sequence) divergence between species and homologues. For example, Cas1 homologues can comprise less than 10% primary sequence identity between homologues. Some Cas genes can comprise homologous secondary and/or tertiary structures. For example, despite extreme sequence divergence, many members of the Cash family of CRISPR proteins comprise a N-terminal ferredoxin-like fold. Cas genes can be named according to the organism from which they are derived. For example, Cas genes in Staphylococcus epidermidis can be referred to as Csm-type, Cas genes in Streptococcus thermophilus can be referred to as Csn-type, and Cas genes in Pyrococcus furiosus can be referred to as Cmr-type.

Integration

The integration stage of CRISPR system can refer to the ability of the CRISPR locus to integrate new spacers into the crRNA array upon being infected by a foreign invader. Acquisition of the foreign invader spacers can help confer immunity to subsequent attacks by the same foreign invader. Integration can occur at the leader end of the CRISPR locus. Cas proteins (e.g., Cas1 and Cas2) can be involved in integration of new spacer sequences. Integration can proceed similarly for some types of CRISPR systems (e.g., Type I-III).

Biogenesis

Mature crRNAs can be processed from a longer polycistronic CRISPR locus transcript (i.e., pre-crRNA array). A pre-crRNA array can comprise a plurality of crRNAs. The repeats in the pre-crRNA array can be recognized by a Cas genes. Cas genes can bind to the repeats and cleave the repeats. This action can liberate the plurality of crRNAs. crRNAs can be subjected to further events to produce the mature crRNA form such as trimming (e.g., with an exonuclese). A crRNA may comprise all, some, or none of the CRISPR repeat sequence.

Interference

Interference can refer to the stage in the CRISPR system that is functionally responsible for combating infection by a foreign invader. CRISPR interference can follow a similar mechanism to RNA interference (RNAi (e.g., wherein a target RNA is targeted (e.g., hybridized) by a short interfering RNA (siRNA)), which can result in target RNA degradation and/or destabilization. CRISPR systems can perform interference of a target nucleic acid by coupling crRNAs and Cas genes, thereby forming CRISPR ribonucleoproteins (crRNPs). crRNA of the crRNP can guide the crRNP to foreign invader nucleic acid, (e.g., by recognizing the foreign invader nucleic acid through hybridization). Hybridized target foreign invader nucleic acid-crRNA units can be subjected to cleavage by Cas proteins. Target nucleic acid interference may require a spacer adjacent motif (PAM) in a target nucleic acid.

Types of CRISPR Systems

There can be four types of CRISPR systems: Type I, Type II, Type III, and Type U. More than one CRISPR type system can be found in an organism. CRISPR systems can be complementary to each other, and/or can lend functional units in trans to facilitate CRISPR locus processing.

Type I CRISPR Systems

crRNA biogenesis in Type I CRISPR systems can comprise endoribonuclease cleavage of repeats in the pre-crRNA array, which can result in a plurality of crRNAs. crRNAs of Type I systems may not be subjected to crRNA trimming. A crRNA can be processed from a pre-crRNA array by a multi-protein complex called CASCADE (originating from CRISPR-associated complex for antiviral defense). CASCADE can comprise protein subunits (e.g., CasA-CasE). Some of the subunits can be members of the Repeat Associated Mysterious Protein (RAMP) superfamily (e.g., Cas5 and Cas6 families). The CASCADE-crRNA complex (i.e., interference complex) can recognize target nucleic acid through hybridization of the crRNA with the target nucleic acid. The CASCADE interference complex can recruit the Cas3 helicase/nuclease which can act in trans to facilitate cleavage of target nucleic acid. The Cas3 nuclease can cleave target nucleic acid (e.g., with its HD nuclease domain). Target nucleic acid in a Type I CRISPR system can comprise a PAM. Target nucleic acid in a Type I CRISPR system can be DNA.

Type I systems can be further subdivided by their species of origin. Type I systems can comprise: Types IA (Aeropyrum pernix or CASS5); IB (Thermotoga neapolitana-Haloarcula marismortui or CASS7); IC (Desulfovibrio vulgaris or CASS1); ID; IE (Escherichia coli or CASS2); and IF (Yersinia pestis or CASS3) subfamilies.

Type II CRISPR Systems

crRNA biogenesis in a Type II CRISPR system can comprise a trans-activating CRISPR RNA (tracrRNA). A tracrRNA can be modified by endogenous RNaseIII. The tracrRNA of the complex can hybridize to a crRNA repeat in the pre-crRNA array. Endogenous RnaseIII can be recruited to cleave the pre-crRNA. Cleaved crRNAs can be subjected to exoribonuclease trimming to produce the mature crRNA form (e.g., 5′ trimming). The tracrRNA can remain hybridized to the crRNA. The tracrRNA and the crRNA can associate with a site-directed polypeptide (e.g., Cas9). The crRNA of the crRNA-tracrRNA-Cas9 complex can guide the complex to a target nucleic acid to which the crRNA can hybridize. Hybridization of the crRNA to the target nucleic acid can activate Cas9 for target nucleic acid cleavage. Target nucleic acid in a Type II CRISPR system can comprise a PAM. In some embodiments, a PAM is essential to facilitate binding of a site-directed polypeptide (e.g., Cas9) to a target nucleic acid. Type II systems can be further subdivided into II-A (Nmeni or CASS4) and II-B (Nmeni or CASS4). CRISPR systems are the subject of active research and new classifications and nomenclatures appear in the art from time-to-time. Classification systems listed here will be understood by one in the art to change from time-to-time and to define related sequences more thoroughly.

Type III CRISPR Systems

crRNA biogenesis in Type III CRISPR systems can comprise a step of endoribonuclease cleavage of repeats in the pre-crRNA array, which can result in a plurality of crRNAs. Repeats in the Type III CRISPR system can be unstructured single-stranded regions. Repeats can be recognized and cleaved by a member of the RAMP superfamily of endoribonucleases (e.g., Cas6). crRNAs of Type III (e.g., Type III-B) systems may be subjected to crRNA trimming (e.g., 3′ trimming). Type III systems can comprise a polymerase-like protein (e.g., Cas10). Cas10 can comprise a domain homologous to a palm domain.

Type III systems can process pre-crRNA with a complex comprising a plurality of RAMP superfamily member proteins and one or more CRISPR polymerase-like proteins. Type III systems can be divided into III-A and III-B. An interference complex of the Type III-A system (i.e., Csm complex) can target plasmid nucleic acid. Cleavage of the plasmid nucleic acid can occur with the HD nuclease domain of a polymerase-like protein in the complex. An interference complex of the Type III-B system (i.e., Cmr complex) can target RNA.

Type U CRISPR Systems

Type U CRISPR systems may not comprise the signature genes of either of the Type I-III CRISPR systems (e.g., Cas3, Cas9, Cas6, Cas1, Cas2). Examples of Type U CRISPR Cas genes can include, but are not limited to, Csf1, Csf2, Csf3, Csf4. Type U Cas genes may be very distant homologues of Type I-III Cas genes. For example, Csf3 may be highly diverged but functionally similar to Cas5 family members. A Type U system may function complementarily in trans with a Type I-III system. In some instances, Type U systems may not be associated with processing CRISPR arrays. Type U systems may represent an alternative foreign invader defense system.

RAMP Superfamily

Repeat Associated Mysterious Proteins (RAMP proteins) can be characterized by a protein fold comprising a βαββαβ [beta-alpha-beta-beta-alpha-beta] motif of β-strands (β) and α-helices (α). A RAMP protein can comprise an RNA recognition motif (RRM) (which can comprise a ferredoxin or ferredoxin-like fold). RAMP proteins can comprise an N-terminal RRM. The C-terminal domain of RAMP proteins can vary, but can also comprise an RRM. RAMP family members can recognize structured and/or unstructured nucleic acid. RAMP family members can recognize single-stranded and/or double-stranded nucleic acid. RAMP proteins can be involved in the biogenesis and/or the interference stage of CRISPR Type I and Type III systems. RAMP superfamily members can comprise members of the Cas7, Cas6, and Cas5 families. RAMP superfamily members can be endoribonucleases.

RRM domains in the RAMP superfamily can be extremely divergent. RRM domains can comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% sequence or structural homology to a wild type exemplary RRM domain (e.g., an RRM domain from Cas7). RRM domains can comprise at most about 5%, at most about 10%, at most about 15%, at most about 20%, at most about 25%, at most about 30%, at most about 35%, at most about 40%, at most about 45%, at most about 50%, at most about 55%, at most about 60%, at most about 65%, at most about 70%, at most about 75%, at most about 80%, at most about 85%, at most about 90%, at most about 95%, or 100% sequence or structural homology to a wild type exemplary RRM domain (e.g., an RRM domain from Cas7).

Cas7 Family

Cas7 family members can be a subclass of RAMP family proteins. Cas7 family proteins can be categorized in Type I CRISPR systems. Cas7 family members may not comprise a glycine rich loop that is familiar to some RAMP family members. Cas7 family members can comprise one RRM domain. Cas7 family members can include, but are not limited to, Cas7 (COG1857), Cas7 (COG3649), Cas7 (CT1975), Csy3, Csm3, Cmr6, Csm5, Cmr4, Cmr1, Csf2, and Csc2.

Cas6 Family

The Cas6 family can be a RAMP subfamily. Cas6 family members can comprise two RNA recognition motif (RRM)-like domains. A Cas6 family member (e.g., Cas6f) can comprise a N-terminal RRM domain and a distinct C-terminal domain that may show weak sequence similarity or structural homology to an RRM domain. Cas6 family members can comprise a catalytic histidine that may be involved in endoribonuclease activity. A comparable motif can be found in Cas5 and Cas7 RAMP families. Cas6 family members can include, but are not limited to, Cas6, Cas6e, Cas6f (e.g., Csy4).

Cas5 Family

The Cas5 family can be a RAMP subfamily. The Cas5 family can be divided into two subgroups: one subgroup that can comprise two RRM domains, and one subgroup that can comprise one RRM domain. Cas5 family members can include, but are not limited to, Csm4, Csx10, Cmr3, Cas5, Cas5(BH0337), Csy2, Csc1, Csf3.

Cas Genes

Exemplary CRISPR Cas genes can include Cas1, Cas2, Cas3′ (Cas3-prime), Cas3″ (Cas3-double prime), Cas4, Cas5, Cas6, Cas6e (formerly referred to as CasE, Cse3), Cas6f (i.e., Csy4), Cas7, Cas8a1, Cas8a2, Cas8b, Cas8c, Cas9, Cas10, Cas10d, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4. Table 1 provides an exemplary categorization of CRISPR Cas genes by CRISPR system type.

The CRISPR-Cas gene naming system has undergone extensive rewriting since the Cas genes were discovered. For the purposes of this application, Cas gene names used herein are based on the naming system outlined in Makarova et al. Evolution and classification of the CRISPR-Cas systems. Nature Reviews Microbiology. 2011 June; 9(6): 467-477. Doi:10.1038/nrmicro2577.

TABLE 1
Exemplary classification of CRISPR Cas genes by CRISPR Type

| System type or | |
|---|---|
| subtype | Gene Name |
| Type I | cas1, cas2, cas3′ |
| Type II | cas1, cas2, cas9 |
| Type III | cas1, cas2, cas10 |
| Subtype I-A | cas3″, cas4, cas5, |
| | cas6, cas7, cas8a1, |
| | cas8a2, csa5 |
| Subtype I-B | cas3″, cas4, cas5, |
| | cas6, cas7, cas8b |
| Subtype I-C | cas4, cas5, cas7, |
| | cas8c |
| Subtype I-D | cas4, cas6, cas10d, |
| | csc1, csc2 |
| Subtype I-E | cas5, cas6e, cas7, |
| | cse1, cse2 |
| Subtype I-F | cas6f, csy1, csy2, |
| | csy3 |
| Subtype II-A | csn2 |
| Subtype II-B | cas4 |
| Subtype III-A | cas6, csm2, csm3, |
| | csm4, csm5, csm6 |
| Subtype III-B | cas6, cmr1, cmr3, |
| | cmr4, cmr5, cmr6 |
| Subtype I-U | csb1, csb2, csb3, |
| | csx17, csx14, csx10 |
| Subtype III-U | csx16, csaX, csx3, |
| | csx1 |
| Unknown | csx15 |
| Type U | csf1, csf2, csf3, |
| | csf4 |

Site-Directed Polypeptides

A site-directed polypeptide can be a polypeptide that can bind to a target nucleic acid. A site-directed polypeptide can be a nuclease.

A site-directed polypeptide can comprise a nucleic acid-binding domain. The nucleic acid-binding domain can comprise a region that contacts a nucleic acid. A nucleic acid-binding domain can comprise a nucleic acid. A nucleic acid-binding domain can comprise a proteinaceous material. A nucleic acid-binding domain can comprise nucleic acid and a proteinaceous material. A nucleic acid-binding domain can comprise RNA. There can be a single nucleic acid-binding domain. Examples of nucleic acid-binding domains can include, but are not limited to, a helix-turn-helix domain, a zinc finger domain, a leucine zipper (bZIP) domain, a winged helix domain, a winged helix turn helix domain, a helix-loop-helix domain, a HMG-box domain, a Wor3 domain, an immunoglobulin domain, a B3 domain, a TALE domain, a RNA-recognition motif domain, a double-stranded RNA-binding motif domain, a double-stranded nucleic acid binding domain, a single-stranded nucleic acid binding domains, a KH domain, a PUF domain, a RGG box domain, a DEAD/DEAH box domain, a PAZ domain, a Piwi domain, and a cold-shock domain.

A nucleic acid-binding domain can be a domain of an Argonaute protein. An Argonaute protein can be a eukaryotic Argonaute or a prokaryotic Argonaute. An Argonaute protein can bind RNA, DNA, or both RNA and DNA. An Argonaute protein can cleaved RNA, or DNA, or both RNA and DNA. In some instances, an Argonaute protein binds a DNA and cleaves a target DNA.

In some instances, two or more nucleic acid-binding domains can be linked together. Linking a plurality of nucleic acid-binding domains together can provide increased polynucleotide targeting specificity. Two or more nucleic acid-binding domains can be linked via one or more linkers. The linker can be a flexible linker. Linkers can comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length. Linkers can comprise at least 5%, 10%, 15%, 20%, 25%, 30%, 35%, 40%, 45%, 50%, 55%, 60%, 65%, 70%, 75%, 80%, 85%, 90%, 95%, or 100% glycine content. Linkers can comprise at most 5%, 10%, 15%, 20%, 25%, 30%, 35%, 40%, 45%, 50%, 55%, 60%, 65%, 70%, 75%, 80%, 85%, 90%, 95%, or 100% glycine content. Linkers can comprise at least 5%, 10%, 15%, 20%, 25%, 30%, 35%, 40%, 45%, 50%, 55%, 60%, 65%, 70%, 75%, 80%, 85%, 90%, 95%, or 100% serine content. Linkers can comprise at most 5%, 10%, 15%, 20%, 25%, 30%, 35%, 40%, 45%, 50%, 55%, 60%, 65%, 70%, 75%, 80%, 85%, 90%, 95%, or 100% serine content.

Nucleic acid-binding domains can bind to nucleic acid sequences. Nucleic acid binding domains can bind to nucleic acids through hybridization. Nucleic acid-binding domains can be engineered (e.g. engineered to hybridize to a sequence in a genome). A nucleic acid-binding domain can be engineered by molecular cloning techniques (e.g., directed evolution, site-specific mutation, and rational mutagenesis).

A site-directed polypeptide can comprise a nucleic acid-cleaving domain. The nucleic acid-cleaving domain can be a nucleic acid-cleaving domain from any nucleic acid-cleaving protein. The nucleic acid-cleaving domain can originate from a nuclease. Suitable nucleic acid-cleaving domains include the nucleic acid-cleaving domain of endonucleases (e.g., AP endonuclease, RecBCD enonuclease, T7 endonuclease, T4 endonuclease IV, Bal 31 endonuclease, Endonucleasel (endo I), Micrococcal nuclease, Endonuclease II (endo VI, exo III)), exonucleases, restriction nucleases, endoribonucleases, exoribonucleases, RNases (e.g., RNAse I, II, or III). In some instances, the nucleic acid-cleaving domain can originate from the FokI endonuclease. A site-directed polypeptide can comprise a plurality of nucleic acid-cleaving domains. Nucleic acid-cleaving domains can be linked together. Two or more nucleic acid-cleaving domains can be linked via a linker. In some embodiments, the linker can be a flexible linker Linkers can comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length. In some embodiments, a site-directed polypeptide can comprise the plurality of nucleic acid-cleaving domains.

A site-directed polypeptide (e.g., Cas9, Argonaute) can comprise two or more nuclease domains. Cas9 can comprise a HNH or FINE-like nuclease domain and/or a RuvC or RuvC-like nuclease domain. HNH or FINE-like domains can comprise a McrA-like fold. HNH or FINE-like domains can comprise two antiparallel β-strands and an α-helix. HNH or FINE-like domains can comprise a metal binding site (e.g., divalent cation binding site). HNH or HNH-like domains can cleave one strand of a target nucleic acid (e.g., complementary strand of the crRNA targeted strand). Proteins that comprise an HNH or HNH-like domain can include endonucleases, clicins, restriction endonucleases, transposases, and DNA packaging factors.

RuvC or RuvC-like domains can comprise an RNaseH or RNaseH-like fold. RuvC/RNaseH domains can be involved in a diverse set of nucleic acid-based functions including acting on both RNA and DNA. The RNaseH domain can comprise 5 β-strands surrounded by a plurality of α-helices. RuvC/RNaseH or RuvC/RNaseH-like domains can comprise a metal binding site (e.g., divalent cation binding site). RuvC/RNaseH or RuvC/RNaseH-like domains can cleave one strand of a target nucleic acid (e.g., non-complementary strand of the crRNA targeted strand). Proteins that comprise a RuvC, RuvC-like, or RNaseH-like domain can include RNaseH, RuvC, DNA transposases, retroviral integrases, and Argonaut proteins).

The site-directed polypeptide can be an endoribonuclease. The site-directed polypeptide can be an enzymatically inactive site-directed polypeptide. The site-directed polypeptide can be a conditionally enzymatically inactive site-directed polypeptide.

Site-directed polypeptides can introduce double-stranded breaks or single-stranded breaks in nucleic acid, (e.g. genomic DNA). The double-stranded break can stimulate a cell's endogenous DNA-repair pathways (e.g. homologous recombination and non-homologous end joining (NHEJ) or alternative non-homologous end joining (A-NHEJ)). NHEJ can repair cleaved target nucleic acid without the need for a homologous template. This can result in deletions of the target nucleic acid. Homologous recombination (HR) can occur with a homologous template. The homologous template can comprise sequences that are homologous to sequences flanking the target nucleic acid cleavage site. After a target nucleic acid is cleaved by a site-directed polypeptide the site of cleavage can be destroyed (e.g., the site may not be accessible for another round of cleavage with the original nucleic acid-targeting nucleic acid and site-directed polypeptide).

In some cases, homologous recombination can insert an exogenous polynucleotide sequence into the target nucleic acid cleavage site. An exogenous polynucleotide sequence can be called a donor polynucleotide. In some instances of the methods of the disclosure the donor polynucleotide, a portion of the donor polynucleotide, a copy of the donor polynucleotide, or a portion of a copy of the donor polynucleotide can be inserted into the target nucleic acid cleavage site. A donor polynucleotide can be an exogenous polynucleotide sequence. A donor polynucleotide can be a sequence that does not naturally occur at the target nucleic acid cleavage site. A vector can comprise a donor polynucleotide. The modifications of the target DNA due to NHEJ and/or HR can lead to, for example, mutations, deletions, alterations, integrations, gene correction, gene replacement, gene tagging, transgene insertion, nucleotide deletion, gene disruption, and/or gene mutation. The process of integrating non-native nucleic acid into genomic DNA can be referred to as genome engineering.

In some cases, the site-directed polypeptide can comprise an amino acid sequence having at most 10%, at most 15%, at most 20%, at most 30%, at most 40%, at most 50%, at most 60%, at most 70%, at most 75%, at most 80%, at most 85%, at most 90%, at most 95%, at most 99%, or 100%, amino acid sequence identity to a wild type exemplary site-directed polypeptide (e.g., Cas9 from S. pyogenes).

In some cases, the site-directed polypeptide can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100%, amino acid sequence identity to a wild type exemplary site-directed polypeptide (e.g., Cas9 from S. pyogenes).

In some cases, the site-directed polypeptide can comprise an amino acid sequence having at most 10%, at most 15%, at most 20%, at most 30%, at most 40%, at most 50%, at most 60%, at most 70%, at most 75%, at most 80%, at most 85%, at most 90%, at most 95%, at most 99%, or 100%, amino acid sequence identity to the nuclease domain of a wild type exemplary site-directed polypeptide (e.g., Cas9 from S. pyogenes).

A site-directed polypeptide can comprise at least 70, 75, 80, 85, 90, 95, 97, 99, or 100% identity to wild-type site-directed polypeptide (e.g., Cas9 from S. pyogenes) over 10 contiguous amino acids. A site-directed polypeptide can comprise at most 70, 75, 80, 85, 90, 95, 97, 99, or 100% identity to wild-type site-directed polypeptide (e.g., Cas9 from S. pyogenes) over 10 contiguous amino acids. A site-directed polypeptide can comprise at least 70, 75, 80, 85, 90, 95, 97, 99, or 100% identity to a wild-type site-directed polypeptide (e.g., Cas9 from S. pyogenes) over 10 contiguous amino acids in a HNH nuclease domain of the site-directed polypeptide. A site-directed polypeptide can comprise at most 70, 75, 80, 85, 90, 95, 97, 99, or 100% identity to a wild-type site-directed polypeptide (e.g., Cas9 from S. pyogenes) over 10 contiguous amino acids in a HNH nuclease domain of the site-directed polypeptide. A site-directed polypeptide can comprise at least 70, 75, 80, 85, 90, 95, 97, 99, or 100% identity to a wild-type site-directed polypeptide (e.g., Cas9 from S. pyogenes) over 10 contiguous amino acids in a RuvC nuclease domain of the site-directed polypeptide. A site-directed polypeptide can comprise at most 70, 75, 80, 85, 90, 95, 97, 99, or 100% identity to a wild-type site-directed polypeptide (e.g., Cas9 from S. pyogenes) over 10 contiguous amino acids in a RuvC nuclease domain of the site-directed polypeptide.

In some cases, the site-directed polypeptide can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100%, amino acid sequence identity to the nuclease domain of a wild type exemplary site-directed polypeptide (e.g., Cas9 from S. pyogenes).

The site-directed polypeptide can comprise a modified form of a wild type exemplary site-directed polypeptide. The modified form of the wild type exemplary site-directed polypeptide can comprise an amino acid change (e.g., deletion, insertion, or substitution) that reduces the nucleic acid-cleaving activity of the site-directed polypeptide. For example, the modified form of the wild type exemplary site-directed polypeptide can have less than less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide (e.g., Cas9 from S. pyogenes). The modified form of the site-directed polypeptide can have no substantial nucleic acid-cleaving activity. When a site-directed polypeptide is a modified form that has no substantial nucleic acid-cleaving activity, it can be referred to as “enzymatically inactive.”

The modified form of the wild type exemplary site-directed polypeptide can have more than 90%, more than 80%, more than 70%, more than 60%, more than 50%, more than 40%, more than 30%, more than 20%, more than 10%, more than 5%, or more than 1% of the nucleic acid-cleaving activity of the wild-type exemplary site-directed polypeptide (e.g., Cas9 from S. pyogenes).

The modified form of the site-directed polypeptide can comprise a mutation. The modified form of the site-directed polypeptide can comprise a mutation such that it can induce a single stranded break (SSB) on a target nucleic acid (e.g., by cutting only one of the sugar-phosphate backbones of the target nucleic acid). The mutation can result in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide (e.g., Cas9 from S. pyogenes). The mutation can result in one or more of the plurality of nucleic acid-cleaving domains retaining the ability to cleave the complementary strand of the target nucleic acid but reducing its ability to cleave the non-complementary strand of the target nucleic acid. The mutation can result in one or more of the plurality of nucleic acid-cleaving domains retaining the ability to cleave the non-complementary strand of the target nucleic acid but reducing its ability to cleave the complementary strand of the target nucleic acid. For example, residues in the wild type exemplary S. pyogenes Cas9 polypeptide such as Asp10, His840, Asn854 and Asn856 can be mutated to inactivate one or more of the plurality of nucleic acid-cleaving domains (e.g., nuclease domains). The residues to be mutated can correspond to residues Asp10, His840, Asn854 and Asn856 in the wild type exemplary S. pyogenes Cas9 polypeptide (e.g., as determined by sequence and/or structural alignment). Non-limiting examples of mutations can include D10A, H840A, N854A or N856A. One skilled in the art will recognize that mutations other than alanine substitutions are suitable.

A D10A mutation can be combined with one or more of H840A, N854A, or N856A mutations to produce a site-directed polypeptide substantially lacking DNA cleavage activity. A H840A mutation can be combined with one or more of Dl OA, N854A, or N856A mutations to produce a site-directed polypeptide substantially lacking DNA cleavage activity. A N854A mutation can be combined with one or more of H840A, Dl OA, or N856A mutations to produce a site-directed polypeptide substantially lacking DNA cleavage activity. A N856A mutation can be combined with one or more of H840A, N854A, or Dl OA mutations to produce a site-directed polypeptide substantially lacking DNA cleavage activity. Site-directed polypeptides that comprise one substantially inactive nuclease domain can be referred to as nickases.

Mutations of the disclosure can be produced by site-directed mutation. Mutations can include substitutions, additions, and deletions, or any combination thereof. In some instances, the mutation converts the mutated amino acid to alanine. In some instances, the mutation converts the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagines, glutamine, histidine, lysine, or arginine). The mutation can convert the mutated amino acid to a non-natural amino acid (e.g., selenomethionine). The mutation can convert the mutated amino acid to amino acid mimics (e.g., phosphomimics). The mutation can be a conservative mutation. For example, the mutation can convert the mutated amino acid to amino acids that resemble the size, shape, charge, polarity, conformation, and/or rotamers of the mutated amino acids (e.g., cysteine/serine mutation, lysine/asparagine mutation, histidine/phenylalanine mutation).

In some instances, the site-directed polypeptide (e.g., variant, mutated, enzymatically inactive and/or conditionally enzymatically inactive site-directed polypeptide) can target nucleic acid. The site-directed polypeptide (e.g., variant, mutated, enzymatically inactive and/or conditionally enzymatically inactive endoribonuclease) can target RNA. Site-directed polypeptides that can target RNA can include members of other CRISPR subfamilies such as Cas6 and Cas5.

The site-directed polypeptide can comprise one or more non-native sequences (e.g., a fusion).

Codon-Optimization

A polynucleotide encoding a site-directed polypeptide and/or an endoribonuclease can be codon-optimized. This type of optimization can entail the mutation of foreign-derived (e.g., recombinant) DNA to mimic the codon preferences of the intended host organism or cell while encoding the same protein. Thus, the codons can be changed, but the encoded protein remains unchanged. For example, if the intended target cell was a human cell, a human codon-optimized polynucleotide Cas9 could be used for producing a suitable site-directed polypeptide. As another non-limiting example, if the intended host cell were a mouse cell, then a mouse codon-optimized polynucleotide encoding Cas9 could be a suitable site-directed polypeptide. A polynucleotide encoding a site-directed polypeptide can be codon optimized for many host cells of interest. A host cell can be a cell from any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc. Codon optimization may not be required. In some instances, codon optimization can be preferable.

Nucleic Acid-Targeting Nucleic Acid

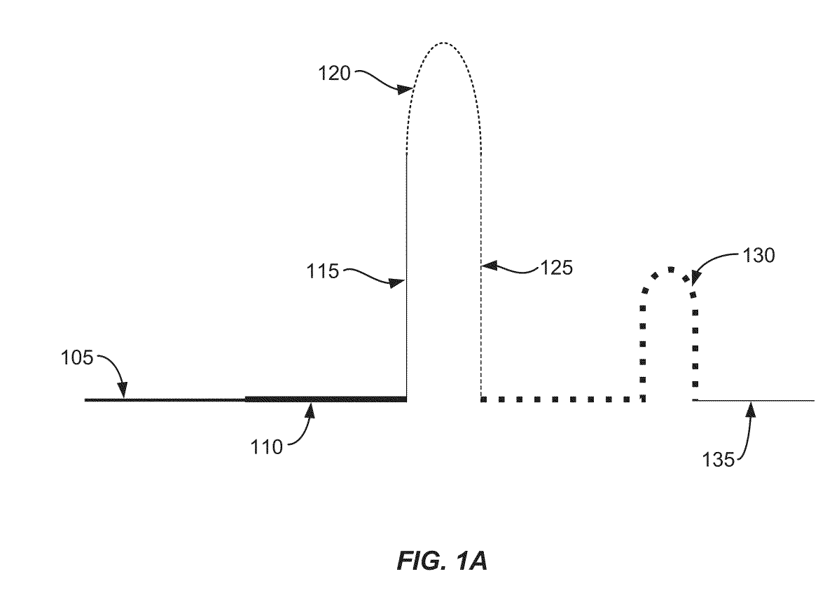

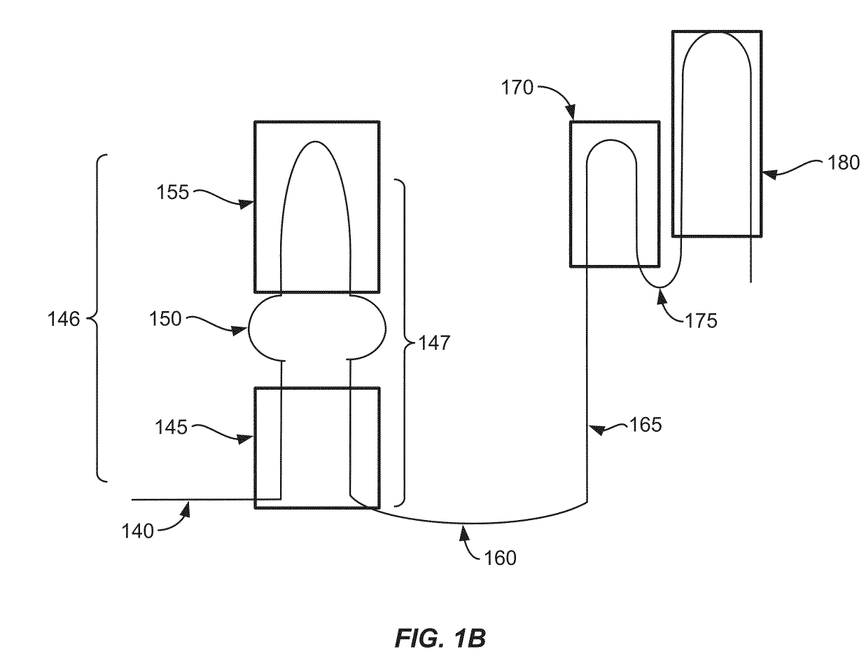

The present disclosure provides for a nucleic acid-targeting nucleic acid that can direct the activities of an associated polypeptide (e.g., a site-directed polypeptide) to a specific target sequence within a target nucleic acid. The nucleic acid-targeting nucleic acid can comprise nucleotides. The nucleic acid-targeting nucleic acid can be RNA. A nucleic acid-targeting nucleic acid can comprise a single guide nucleic acid-targeting nucleic acid. A nucleic acid-targeting nucleic acid can comprise a crRNA hybridized to a tracrRNA. An exemplary single guide nucleic acid-targeting nucleic acid is depicted in FIG. 1A. The spacer extension 105 and the tracrRNA extension 135 can comprise elements that can contribute additional functionality (e.g., stability) to the nucleic acid-targeting nucleic acid. In some embodiments the spacer extension 105 and the tracrRNA extension 135 are optional. A spacer sequence 110 can comprise a sequence that can hybridize to a target nucleic acid sequence. The spacer sequence 110 can be a variable portion of the nucleic acid-targeting nucleic acid. The sequence of the spacer sequence 110 can be engineered to hybridize to the target nucleic acid sequence. The CRISPR repeat 115 (i.e. referred to in this exemplary embodiment as a minimum CRISPR repeat) can comprise nucleotides that can hybridize to a tracrRNA sequence 125 (i.e. referred to in this exemplary embodiment as a minimum tracrRNA sequence). The minimum CRISPR repeat 115 and the minimum tracrRNA sequence 125 can interact, the interacting molecules comprising a base-paired, double-stranded structure. Together, the minimum CRISPR repeat 115 and the minimum tracrRNA sequence 125 form a stem loop duplex structure and can facilitate binding to the site-directed polypeptide. The minimum CRISPR repeat 115 and the minimum tracrRNA sequence 125 can be linked together to form a hairpin structure through the single guide connector 120. The 3′ tracrRNA sequence 130 can comprise a protospacer adjacent motif recognition sequence. The 3′ tracrRNA sequence 130 can be identical or similar to part of a tracrRNA sequence. In some embodiments, the 3′ tracrRNA sequence 130 can comprise one or more hairpins.

In some embodiments, a nucleic acid-targeting nucleic acid can comprise a single guide nucleic acid-targeting nucleic acid as depicted in FIG. 1B. A nucleic acid-targeting nucleic acid can comprise a spacer sequence 140. A spacer sequence 140 can comprise a sequence that can hybridize to the target nucleic acid sequence. The spacer sequence 140 can be a variable portion of the nucleic acid-targeting nucleic acid. The spacer sequence 140 can be 5′ of a first duplex 145. The first duplex 145 comprises a region of hybridization between a minimum CRISPR repeat 146 and minimum tracrRNA sequence 147. The first duplex 145 can be interrupted by a bulge 150. The bulge 150 can comprise unpaired nucleotides. The bulge 150 can facilitate the recruitment of a site-directed polypeptide to the nucleic acid-targeting nucleic acid. The bulge 150 can be followed by a first stem 155. The first stem 155 comprises a linker sequence linking the minimum CRISPR repeat 146 and the minimum tracrRNA sequence 147. The last paired nucleotide at the 3′ end of the first duplex 145 can be connected to a second linker sequence 160. The second linker 160 can comprise a nexus. The second linker 160 can link the first duplex 145 to a mid-tracrRNA 165. The mid-tracrRNA 165 can, in some embodiments, comprise one or more hairpin regions. For example the mid-tracrRNA 165 can comprise a second stem 170 and a third stem 175.

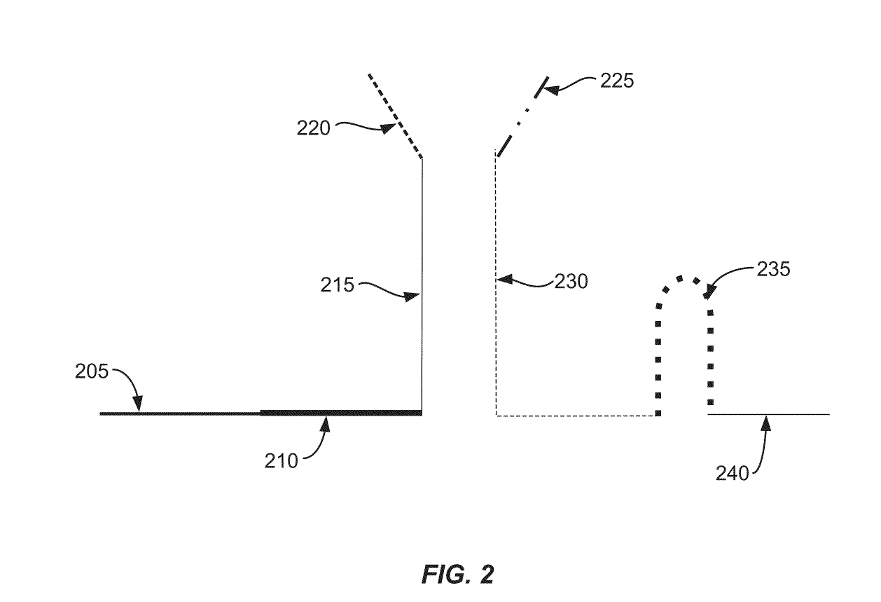

In some embodiments, the nucleic acid-targeting nucleic acid can comprise a double guide nucleic acid structure. FIG. 2 depicts an exemplary double guide nucleic acid-targeting nucleic acid structure. Similar to the single guide nucleic acid structure of FIG. 1, the double guide nucleic acid structure can comprise a spacer extension 205, a spacer 210, a minimum CRISPR repeat 215, a minimum tracrRNA sequence 230, a 3′ tracrRNA sequence 235, and a tracrRNA extension 240. However, a double guide nucleic acid-targeting nucleic acid may not comprise the single guide connector 120. Instead the minimum CRISPR repeat sequence 215 can comprise a 3′ CRISPR repeat sequence 220 which can be similar or identical to part of a CRISPR repeat. Similarly, the minimum tracrRNA sequence 230 can comprise a 5′ tracrRNA sequence 225 which can be similar or identical to part of a tracrRNA. The double guide RNAs can hybridize together via the minimum CRISPR repeat 215 and the minimum tracrRNA sequence 230.

In some embodiments, the first segment (i.e., nucleic acid-targeting segment) can comprise the spacer extension (e.g., 105/205) and the spacer (e.g., 110/210). The nucleic acid-targeting nucleic acid can guide the bound polypeptide to a specific nucleotide sequence within target nucleic acid via the above mentioned nucleic acid-targeting segment.

In some embodiments, the second segment (i.e., protein binding segment) can comprise the minimum CRISPR repeat (e.g., 115/215), the minimum tracrRNA sequence (e.g., 125/230), the 3′ tracrRNA sequence (e.g., 130/235), and/or the tracrRNA extension sequence (e.g., 135/240). The protein-binding segment of a nucleic acid-targeting nucleic acid can interact with a site-directed polypeptide. The protein-binding segment of a nucleic acid-targeting nucleic acid can comprise two stretches of nucleotides that that can hybridize to one another. The nucleotides of the protein-binding segment can hybridize to form a double-stranded nucleic acid duplex. The double-stranded nucleic acid duplex can be RNA. The double-stranded nucleic acid duplex can be DNA.

In some instances, a nucleic acid-targeting nucleic acid can comprise, in the order of 5′ to 3′, a spacer extension, a spacer, a minimum CRISPR repeat, a single guide connector, a minimum tracrRNA, a 3′ tracrRNA sequence, and a tracrRNA extension. In some instances, a nucleic acid-targeting nucleic acid can comprise, a tracrRNA extension, a 3′tracrRNA sequence, a minimum tracrRNA, a single guide connector, a minimum CRISPR repeat, a spacer, and a spacer extension in any order.

In some instances, a nucleic acid-targeting nucleic acid comprises a spacer, a lower stem, an upper stem, a bulge, a nexus, and one or more 3′ hairpins. The lower stem and the upper stem can be separated by the bulge. The lower stem and the upper stem can comprise a duplex between the minimum CRISPR repeat and the minimum tracrRNA.

A nucleic acid-targeting nucleic acid and a site-directed polypeptide can form a complex. The nucleic acid-targeting nucleic acid can provide target specificity to the complex by comprising a nucleotide sequence that can hybridize to a sequence of a target nucleic acid (e.g., a spacer). In other words, the site-directed polypeptide can be guided to a nucleic acid sequence by virtue of its association with at least the protein-binding segment of the nucleic acid-targeting nucleic acid. The nucleic acid-targeting nucleic acid can direct the activity of a Cas9 protein. The nucleic acid-targeting nucleic acid can direct the activity of an enzymatically inactive Cas9 protein.

Methods of the disclosure can provide for a genetically modified cell. A genetically modified cell can comprise an exogenous nucleic acid-targeting nucleic acid and/or an exogenous nucleic acid comprising a nucleotide sequence encoding a nucleic acid-targeting nucleic acid.

Spacer Extension Sequence

A spacer extension sequence can provide stability and/or provide a location for modifications of a nucleic acid-targeting nucleic acid. A spacer extension sequence or 5′spacer extension sequence can be 5′ to a spacer. A spacer extension sequence can have a length of from about 1 nucleotide to about 400 nucleotides. A spacer extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000 or more nucleotides. A spacer extension sequence can be less than 10 nucleotides in length. A spacer extension sequence can be between 10 and 30 nucleotides in length. A spacer extension sequence can be between 30-70 nucleotides in length.

The spacer extension sequence can comprise a moiety (e.g., a stability control sequence, an endoribonuclease binding sequence, a ribozyme). A moiety can influence the stability of a nucleic acid targeting RNA. A moiety can be a transcriptional terminator segment (i.e., a transcription termination sequence). A moiety of a nucleic acid-targeting nucleic acid can have a total length of from about 10 nucleotides to about 100 nucleotides, from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nucleotides (nt) to about 80 nt, from about 15 nt to about 50 nt, from about 15 nt to about 40 nt, from about 15 nt to about 30 nt or from about 15 nt to about 25 nt. The moiety can be one that can function in a eukaryotic cell. In some cases, the moiety can be one that can function in a prokaryotic cell. The moiety can be one that can function in both a eukaryotic cell and a prokaryotic cell.

Non-limiting examples of suitable moieties can include: 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) a modification or sequence that provides for increased, decreased, and/or controllable stability, or any combination thereof. A spacer extension sequence can comprise a primer binding site, a molecular index (e.g., barcode sequence). The spacer extension sequence can comprise a nucleic acid affinity tag.

Spacer

The nucleic acid-targeting segment of a nucleic acid-targeting nucleic acid can comprise a nucleotide sequence (e.g., a spacer) that can hybridize to a sequence in a target nucleic acid. The spacer of a nucleic acid-targeting nucleic acid can interact with a target nucleic acid in a sequence-specific manner via hybridization (i.e., base pairing). As such, the nucleotide sequence of the spacer may vary and can determine the location within the target nucleic acid that the nucleic acid-targeting nucleic acid and site-directed polypeptide can interact.

The spacer sequence can hybridize to a target nucleic acid that is located 5′ of protospacer adjacent motif (PAM). Different organisms may comprise different PAM sequences. For example, in S. pyogenes, the PAM can be a sequence in the target nucleic acid that comprises the sequence 5′-XRR-3′, where R can be either A or G, where X is any nucleotide and X is immediately 3′ of the target nucleic acid sequence targeted by the spacer sequence.

The target nucleic acid sequence can be 20 nucleotides. The target nucleic acid can be less than 20 nucleotides. The target nucleic acid can be at least 5, 10, 15, 16, 17, 18, 19, 20, 21, 22, 23, 24, 25, 30 or more nucleotides. The target nucleic acid can be at most 5, 10, 15, 16, 17, 18, 19, 20, 21, 22, 23, 24, 25, 30 or more nucleotides. The target nucleic acid sequence can be 20 bases immediately 5′ of the first nucleotide of the PAM. For example, in a sequence comprising 5′-NNNNNNNNNNNNNNNNNNNNXRR-3′, the target nucleic acid can be the sequence that corresponds to the N's, wherein N is any nucleotide.

The nucleic acid-targeting sequence of the spacer that can hybridize to the target nucleic acid can have a length at least about 6 nt. For example, the spacer sequence that can hybridize the target nucleic acid can have a length at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the spacer sequence that can hybridize the target nucleic acid can be 20 nucleotides in length. The spacer that can hybridize the target nucleic acid can be 19 nucleotides in length.

The percent complementarity between the spacer sequence the target nucleic acid can be at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. The percent complementarity between the spacer sequence the target nucleic acid can be at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some cases, the percent complementarity between the spacer sequence and the target nucleic acid can be 100% over the six contiguous 5′-most nucleotides of the target sequence of the complementary strand of the target nucleic acid. In some cases, the percent complementarity between the spacer sequence and the target nucleic acid can be at least 60% over about 20 contiguous nucleotides. In some cases, the percent complementarity between the spacer sequence and the target nucleic acid can be 100% over the fourteen contiguous 5′-most nucleotides of the target sequence of the complementary strand of the target nucleic acid and as low as 0% over the remainder. In such a case, the spacer sequence can be considered to be 14 nucleotides in length. In some cases, the percent complementarity between the spacer sequence and the target nucleic acid can be 100% over the six contiguous 5′-most nucleotides of the target sequence of the complementary strand of the target nucleic acid and as low as 0% over the remainder. In such a case, the spacer sequence can be considered to be 6 nucleotides in length. The target nucleic acid can be more than about 50%, 60%, 70%, 80%, 90%, or 100% complementary to the seed region of the crRNA. The target nucleic acid can be less than about 50%, 60%, 70%, 80%, 90%, or 100% complementary to the seed region of the crRNA.

The spacer segment of a nucleic acid-targeting nucleic acid can be modified (e.g., by genetic engineering) to hybridize to any desired sequence within a target nucleic acid. For example, a spacer can be engineered (e.g., designed, programmed) to hybridize to a sequence in target nucleic acid that is involved in cancer, cell growth, DNA replication, DNA repair, HLA genes, cell surface proteins, T-cell receptors, immunoglobulin superfamily genes, tumor suppressor genes, microRNA genes, long non-coding RNA genes, transcription factors, globins, viral proteins, mitochondrial genes, and the like.

A spacer sequence can be identified using a computer program (e.g., machine readable code). The computer program can use variables such as predicted melting temperature, secondary structure formation, and predicted annealing temperature, sequence identity, genomic context, chromatin accessibility, % GC, frequency of genomic occurrence, methylation status, presence of SNPs, and the like.

Minimum CRISPR Repeat Sequence

A minimum CRISPR repeat sequence can be a sequence at least about 30%, 40%, 50%, 60%, 65%, 70%, 75%, 80%, 85%, 90%, 95%, or 100% sequence identity and/or sequence homology with a reference CRISPR repeat sequence (e.g., crRNA from S. pyogenes). A minimum CRISPR repeat sequence can be a sequence with at most about 30%, 40%, 50%, 60%, 65%, 70%, 75%, 80%, 85%, 90%, 95%, or 100% sequence identity and/or sequence homology with a reference CRISPR repeat sequence (e.g., crRNA from S. pyogenes). A minimum CRISPR repeat can comprise nucleotides that can hybridize to a minimum tracrRNA sequence. A minimum CRISPR repeat and a minimum tracrRNA sequence can form a base-paired, double-stranded structure. Together, the minimum CRISPR repeat and the minimum tracrRNA sequence can facilitate binding to the site-directed polypeptide. A part of the minimum CRISPR repeat sequence can hybridize to the minimum tracrRNA sequence. A part of the minimum CRISPR repeat sequence can be at least about 30%, 40%, 50%, 60%, 65%, 70%, 75%, 80%, 85%, 90%, 95%, or 100% complementary to the minimum tracrRNA sequence. A part of the minimum CRISPR repeat sequence can be at most about 30%, 40%, 50%, 60%, 65%, 70%, 75%, 80%, 85%, 90%, 95%, or 100% complementary to the minimum tracrRNA sequence.

The minimum CRISPR repeat sequence can have a length of from about 6 nucleotides to about 100 nucleotides. For example, the minimum CRISPR repeat sequence can have a length of from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt or from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt. In some embodiments, the minimum CRISPR repeat sequence has a length of approximately 12 nucleotides.

The minimum CRISPR repeat sequence can be at least about 60% identical to a reference minimum CRISPR repeat sequence (e.g., wild type crRNA from S. pyogenes) over a stretch of at least 6, 7, or 8 contiguous nucleotides. The minimum CRISPR repeat sequence can be at least about 60% identical to a reference minimum CRISPR repeat sequence (e.g., wild type crRNA from S. pyogenes) over a stretch of at least 6, 7, or 8 contiguous nucleotides. For example, the minimum CRISPR repeat sequence can be at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum CRISPR repeat sequence over a stretch of at least 6, 7, or 8 contiguous nucleotides.

Minimum tracrRNA Sequence

A minimum tracrRNA sequence can be a sequence with at least about 30%, 40%, 50%, 60%, 65%, 70%, 75%, 80%, 85%, 90%, 95%, or 100% sequence identity and/or sequence homology to a reference tracrRNA sequence (e.g., wild type tracrRNA from S. pyogenes). A minimum tracrRNA sequence can be a sequence with at most about 30%, 40%, 50%, 60%, 65%, 70%, 75%, 80%, 85%, 90%, 95%, or 100% sequence identity and/or sequence homology to a reference tracrRNA sequence (e.g., wild type tracrRNA from S. pyogenes). A minimum tracrRNA sequence can comprise nucleotides that can hybridize to a minimum CRISPR repeat sequence. A minimum tracrRNA sequence and a minimum CRISPR repeat sequence can form a base-paired, double-stranded structure. Together, the minimum tracrRNA sequence and the minimum CRISPR repeat can facilitate binding to the site-directed polypeptide. A part of the minimum tracrRNA sequence can hybridize to the minimum CRISPR repeat sequence. A part of the minimum tracrRNA sequence can be 30%, 40%, 50%, 60%, 65%, 70%, 75%, 80%, 85%, 90%, 95%, or 100% complementary to the minimum CRISPR repeat sequence.

The minimum tracrRNA sequence can have a length of from about 6 nucleotides to about 100 nucleotides. For example, the minimum tracrRNA sequence can have a length of from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt or from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt. In some embodiments, the minimum tracrRNA sequence has a length of approximately 14 nucleotides.

The minimum tracrRNA sequence can be at least about 60% identical to a reference minimum tracrRNA (e.g., wild type, tracrRNA from S. pyogenes) sequence over a stretch of at least 6, 7, or 8 contiguous nucleotides. The minimum tracrRNA sequence can be at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence can be at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum tracrRNA sequence over a stretch of at least 6, 7, or 8 contiguous nucleotides.

The duplex (i.e., first duplex in FIG. 1B) between the minimum CRISPR RNA and the minimum tracrRNA can comprise a double helix. The first base of the first strand of the duplex (e.g., the minimum CRISPR repeat in FIG. 1B) can be a guanine. The first base of the first strand of the duplex (e.g., the minimum CRISPR repeat in FIG. 1B) can be an adenine. The duplex (i.e., first duplex in FIG. 1B) between the minimum CRISPR RNA and the minimum tracrRNA can comprise at least about 1, 2, 3, 4, 5, 6, 7, 8, 9, or 10 or more nucleotides. The duplex (i.e., first duplex in FIG. 1B) between the minimum CRISPR RNA and the minimum tracrRNA can comprise at most about 1, 2, 3, 4, 5, 6, 7, 8, 9, or 10 or more nucleotides.

The duplex can comprise a mismatch. The duplex can comprise at least about 1, 2, 3, 4, or 5 or mismatches. The duplex can comprise at most about 1, 2, 3, 4, or 5 or mismatches. In some instances, the duplex comprises no more than 2 mismatches.

Bulge

A bulge can refer to an unpaired region of nucleotides within the duplex made up of the minimum CRISPR repeat and the minimum tracrRNA sequence. The bulge can be important in the binding to the site-directed polypeptide. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

For example, the bulge can comprise an unpaired purine (e.g., adenine) on the minimum CRISPR repeat strand of the bulge. In some embodiments, a bulge can comprise an unpaired 5′-AAGY-3′ of the minimum tracrRNA sequence strand of the bulge, where Y can be a nucleotide that can form a wobble pairing with a nucleotide on the minimum CRISPR repeat strand.

A bulge on a first side of the duplex (e.g., the minimum CRISPR repeat side) can comprise at least 1, 2, 3, 4, or 5 or more unpaired nucleotides. A bulge on a first side of the duplex (e.g., the minimum CRISPR repeat side) can comprise at most 1, 2, 3, 4, or 5 or more unpaired nucleotides. A bulge on the first side of the duplex (e.g., the minimum CRISPR repeat side) can comprise 1 unpaired nucleotide.

A bulge on a second side of the duplex (e.g., the minimum tracrRNA sequence side of the duplex) can comprise at least 1, 2, 3, 4, 5, 6, 7, 8, 9, or 10 or more unpaired nucleotides. A bulge on a second side of the duplex (e.g., the minimum tracrRNA sequence side of the duplex) can comprise at most 1, 2, 3, 4, 5, 6, 7, 8, 9, or 10 or more unpaired nucleotides. A bulge on a second side of the duplex (e.g., the minimum tracrRNA sequence side of the duplex) can comprise 4 unpaired nucleotides.

Regions of different numbers of unpaired nucleotides on each strand of the duplex can be paired together. For example, a bulge can comprise 5 unpaired nucleotides from a first strand and 1 unpaired nucleotide from a second strand. A bulge can comprise 4 unpaired nucleotides from a first strand and 1 unpaired nucleotide from a second strand. A bulge can comprise 3 unpaired nucleotides from a first strand and 1 unpaired nucleotide from a second strand. A bulge can comprise 2 unpaired nucleotides from a first strand and 1 unpaired nucleotide from a second strand. A bulge can comprise 1 unpaired nucleotide from a first strand and 1 unpaired nucleotide from a second strand. A bulge can comprise 1 unpaired nucleotide from a first strand and 2 unpaired nucleotides from a second strand. A bulge can comprise 1 unpaired nucleotide from a first strand and 3 unpaired nucleotides from a second strand. A bulge can comprise 1 unpaired nucleotide from a first strand and 4 unpaired nucleotides from a second strand. A bulge can comprise 1 unpaired nucleotide from a first strand and 5 unpaired nucleotides from a second strand.

In some instances a bulge can comprise at least one wobble pairing. In some instances, a bulge can comprise at most one wobble pairing. A bulge sequence can comprise at least one purine nucleotide. A bulge sequence can comprise at least 3 purine nucleotides. A bulge sequence can comprise at least 5 purine nucleotides. A bulge sequence can comprise at least one guanine nucleotide. A bulge sequence can comprise at least one adenine nucleotide.

Nexus

The nexus can be located downstream of (i.e., located in the 3′ direction from) the first stem-loop duplex element. An exemplary location of the nexus is illustrated in the single-guide RNA (sgRNA) nucleic acid targeting nucleic acid used to support activity with the S. pyogenes Cas9 protein shown in FIG. 1A (130).

A nexus can start at least about 1, 2, 3, 4, 5, 6, 7, 8, 9, 10, 15, or 20 or more nucleotides 3′ of the last paired nucleotide in the minimum CRISPR repeat and minimum tracrRNA sequence duplex. A nexus can start at most about 1, 2, 3, 4, 5, 6, 7, 8, 9 or 10 or more nucleotides 3′ of the last paired nucleotide in the minimum CRISPR repeat and minimum tracrRNA sequence duplex.

A nexus can comprise at least about 1, 2, 3, 4, 5, 6, 7, 8, 9, 10, 15, or 20 or more consecutive nucleotides. A nexus can comprise at most about 1, 2, 3, 4, 5, 6, 7, 8, 9, 10, 15, or 20 or more consecutive nucleotides.

A nexus can comprise a hairpin. The stem duplex of the hairpin can comprise a dinucleotide duplex (e.g., two stacked base-paired nucleotides). The stem duplex of the hairpin can comprise a tri-nucleotide duplex (e.g., three stacked base-paired nucleotides). The stem duplex of the hairpin can comprise a quattro-nucleotide duplex (e.g., four stacked base-paired nucleotides). The hairpin can comprise a stem loop. The stem loop structure of the hairpin of the nexus can comprise at least 1, 2, 3, 4, 5, 6, 7, 8, 9, or 10 or more nucleotides. The stem loop structure of the hairpin of the nexus can comprise at most 1, 2, 3, 4, 5, 6, 7, 8, 9, or 10 or more nucleotides.

A nexus can be a nucleotide sequence located in the 3′ tracrRNA sequence (i.e., mid-tracrRNA sequence). A nexus can comprise duplexed nucleotides (e.g., nucleotides in a hairpin, hybridized together. For example, a nexus can comprise a CC dinucleotide that is hybridized to a GG dinucleotide in a hairpin duplex of the 3′ tracrRNA sequence (i.e., mid-tracrRNA sequence).

The nexus can interact with nexus interacting regions within the site-directed polypeptide. The nexus can interact with an arginine-rich basic patch in the site-directed polypeptide. The nexus interacting regions can interact with a PAM sequence. The nexus can comprise a stem loop. The nexus can comprise a bulge.

3′ tracrRNA Sequence or Hairpins

As used herein, the terms “3′ tracrRNA sequence” and “hairpins,” “3′ hairpins,” or “hairpins region” can be used interchangeably. A 3′ tracr RNA sequence can be a sequence with at least about 30%, 40%, 50%, 60%, 65%, 70%, 75%, 80%, 85%, 90%, 95%, or 100% sequence identity and/or sequence homology with a reference tracrRNA sequence (e.g., a tracrRNA from S. pyogenes). A 3′tracr RNA sequence can be a sequence with at most about 30%, 40%, 50%, 60%, 65%, 70%, 75%, 80%, 85%, 90%, 95%, or 100% sequence identity and/or sequence homology with a reference tracrRNA sequence (e.g., tracrRNA from S. pyogenes).

The 3′ tracrRNA sequence can have a length of from about 6 nucleotides to about 100 nucleotides. For example, the 3′ tracrRNA sequence can have a length of from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt or from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt. In some embodiments, the 3′ tracrRNA sequence has a length of approximately 14 nucleotides.

The 3′ tracrRNA sequence can be at least about 60% identical to a reference 3′ tracrRNA sequence (e.g., wild type 3′ tracrRNA sequence from S. pyogenes) over a stretch of at least 6, 7, or 8 contiguous nucleotides. For example, the 3′ tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical, to a reference 3′ tracrRNA sequence (e.g., wild type 3′ tracrRNA sequence from S. pyogenes) over a stretch of at least 6, 7, or 8 contiguous nucleotides.

A 3′ tracrRNA sequence can comprise more than one duplexed region (e.g., hairpin, hybridized region). A 3′ tracrRNA sequence can comprise two duplexed regions.

The 3′ tracrRNA sequence can also be referred to as the mid-tracrRNA (See FIG. 1B). The mid-tracrRNA sequence can comprise a stem loop structure. In other words, the mid-tracrRNA sequence can comprise a hairpin that is different than a second or third stems, as depicted in FIG. 1B. A stem loop structure in the mid-tracrRNA (i.e., 3′ tracrRNA) can comprise at least 1, 2, 3, 4, 5, 6, 7, 8, 9, 10, 15 or 20 or more nucleotides. A stem loop structure in the mid-tracrRNA (i.e., 3′ tracrRNA) can comprise at most 1, 2, 3, 4, 5, 6, 7, 8, 9 or 10 or more nucleotides. The stem loop structure can comprise a functional moiety. For example, the stem loop structure can comprise an aptamer, a ribozyme, a protein-interacting hairpin, a CRISPR array, an intron, and an exon. The stem loop structure can comprise at least about 1, 2, 3, 4, or 5 or more functional moieties. The stem loop structure can comprise at most about 1, 2, 3, 4, or 5 or more functional moieties.

Loop

A nucleic acid-targeting nucleic acid of the disclosure can comprise a loop region. The loop region can separate the nexus from the 3′ tracrRNA (e.g., hairpins) sequence. The loop region can refer to consecutive single-stranded nucleotides between the nexus and the hairpins of the 3′ tracrRNA. The 3′ tracrRNA sequence can comprise the loop. The loop region can be at least 1, 2, 3, 4, 5, 6, 7, 8, 9, or 10 or more nucleotides in length. The loop region can be at most 1, 2, 3, 4, 5, 6, 7, 8, 9, or 10 or more nucleotides in length.

A loop region can be a sequence with at least about 30%, 40%, 50%, 60%, 65%, 70%, 75%, 80%, 85%, 90%, 95%, or 100% sequence identity and/or sequence homology with a reference loop region (e.g., a loop region from S. pyogenes). A loop region can be a sequence with at most about 30%, 40%, 50%, 60%, 65%, 70%, 75%, 80%, 85%, 90%, 95%, or 100% sequence identity and/or sequence homology with a reference loop region (e.g., a loop region from S. pyogenes).

tracrRNA Extension Sequence

A tracrRNA extension sequence can provide stability and/or provide a location for modifications of a nucleic acid-targeting nucleic acid. A tracrRNA extension sequence can have a length of from about 1 nucleotide to about 400 nucleotides. A tracrRNA extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 or more nucleotides. A tracrRNA extension sequence can have a length from about 20 to about 5000 or more nucleotides. A tracrRNA extension sequence can have a length of more than 1000 nucleotides. A tracrRNA extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 nucleotides. A tracrRNA extension sequence can have a length of less than 1000 nucleotides. A tracrRNA extension sequence can be less than 10 nucleotides in length. A tracrRNA extension sequence can be between 10 and 30 nucleotides in length. A tracrRNA extension sequence can be between 30-70 nucleotides in length.

The tracrRNA extension sequence can comprise a moiety (e.g., stability control sequence, ribozyme, endoribonuclease binding sequence). A moiety can influence the stability of a nucleic acid targeting RNA. A moiety can be a transcriptional terminator segment (i.e., a transcription termination sequence). A moiety of a nucleic acid-targeting nucleic acid can have a total length of from about 10 nucleotides to about 100 nucleotides, from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nucleotides (nt) to about 80 nt, from about 15 nt to about 50 nt, from about 15 nt to about 40 nt, from about 15 nt to about 30 nt or from about 15 nt to about 25 nt. The moiety can be one that can function in a eukaryotic cell. In some cases, the moiety can be one that can function in a prokaryotic cell. The moiety can be one that can function in both a eukaryotic cell and a prokaryotic cell.

Non-limiting examples of suitable tracrRNA extension moieties include: a 3′ poly-adenylated tail, a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) a modification or sequence that provides for increased, decreased, and/or controllable stability, or any combination thereof. A tracrRNA extension sequence can comprise a primer binding site, a molecular index (e.g., barcode sequence). In some embodiments of the disclosure, the tracrRNA extension sequence can comprise one or more affinity tags.

Single Guide Nucleic Acid

The nucleic acid-targeting nucleic acid can be a single guide nucleic acid. The single guide nucleic acid can be RNA (e.g., sgRNA). A single guide nucleic acid can comprise a linker (i.e. item 120 from FIG. 1A) between the minimum CRISPR repeat sequence and the minimum tracrRNA sequence that can be called a single guide connector sequence.

The single guide connector of a single guide nucleic acid can have a length of from about 3 nucleotides to about 100 nucleotides. For example, the linker can have a length of from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt or from about 3 nt to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a single guide nucleic acid is between 4 and 40 nucleotides. A linker can have a length at least about 100, 500, 1000, 1500, 2000, 2500, 3000, 3500, 4000, 4500, 5000, 5500, 6000, 6500, or 7000 or more nucleotides. A linker can have a length at most about 100, 500, 1000, 1500, 2000, 2500, 3000, 3500, 4000, 4500, 5000, 5500, 6000, 6500, or 7000 or more nucleotides.

The linker sequence can comprise a functional moiety. For example, the linker sequence can comprise an aptamer, a ribozyme, a protein-interacting hairpin, a CRISPR array, an intron, and an exon. The linker sequence can comprise at least about 1, 2, 3, 4, or 5 or morefunctional moieties. The linker sequence can comprise at most about 1, 2, 3, 4, or 5 or more functional moieties.

In some embodiments, the single guide connector can connect the 3′ end of the minimum CRISPR repeat to the 5′ end of the minimum tracrRNA sequence. Alternatively, the single guide connector can connect the 3′ end of the tracrRNA sequence to the 5′end of the minimum CRISPR repeat. That is to say, a single guide nucleic acid can comprise a 5′ DNA-binding segment linked to a 3′ protein-binding segment. A single guide nucleic acid can comprise a 5′ protein-binding segment linked to a 3′ DNA-binding segment.

A nucleic acid-targeting nucleic acid can comprise a spacer extension sequence from 10-5000 nucleotides in length; a spacer sequence of 12-30 nucleotides in length, wherein the spacer is at least 50% complementary to a target nucleic acid; a minimum CRISPR repeat comprising at least 60% identity to a crRNA from a prokaryote (e.g., S. pyogenes) or phage over 6, 7, or 8 contiguous nucleotides and wherein the minimum CRISPR repeat has a length from 5-30 nucleotides; a minimum tracrRNA sequence comprising at least 60% identity to a tracrRNA from a bacterium (e.g., S. pyogenes) over 6, 7, or 8 contiguous nucleotides and wherein the minimum tracrRNA sequence has a length from 5-30 nucleotides; a linker sequence that links the minimum CRISPR repeat and the minimum tracrRNA and comprises a length from 3-5000 nucleotides; a 3′ tracrRNA that comprises at least 60% identity to a tracrRNA from a prokaryote (e.g., S. pyogenes) or phage over 6, 7, or 8 contiguous nucleotides and wherein the 3′ tracrRNA comprises a length from 10-20 nucleotides, and comprises a duplexed region; and/or a tracrRNA extension comprising 10-5000 nucleotides in length, or any combination thereof. This nucleic acid-targeting nucleic acid can be referred to as a single guide nucleic acid-targeting nucleic acid.

A nucleic acid-targeting nucleic acid can comprise a spacer extension sequence from 10-5000 nucleotides in length; a spacer sequence of 12-30 nucleotides in length, wherein the spacer is at least 50% complementary to a target nucleic acid; a duplex comprising 1) a minimum CRISPR repeat comprising at least 60% identity to a crRNA from a prokaryote (e.g., S. pyogenes) or phage over 6 contiguous nucleotides and wherein the minimum CRISPR repeat has a length from 5-30 nucleotides, 2) a minimum tracrRNA sequence comprising at least 60% identity to a tracrRNA from a bacterium (e.g., S. pyogenes) over 6 contiguous nucleotides and wherein the minimum tracrRNA sequence has a length from 5-30 nucleotides, and 3) a bulge wherein the bulge comprises at least 3 unpaired nucleotides on the minimum CRISPR repeat strand of the duplex and at least 1 unpaired nucleotide on the minimum tracrRNA sequence strand of the duplex; a linker sequence that links the minimum CRISPR repeat and the minimum tracrRNA and comprises a length from 3-5000 nucleotides; a 3′ tracrRNA that comprises at least 60% identity to a tracrRNA from a prokaryote (e.g., S. pyogenes) or phage over 6 contiguous nucleotides, wherein the 3′ tracrRNA comprises a length from 10-20 nucleotides and comprises a duplexed region; a nexus that starts from 1-5 nucleotides downstream of the duplex comprising the minimum CRISPR repeat and the minimum tracrRNA, comprises 1-10 nucleotides, can form a hairpin, and is located in the 3′ tracrRNA region; and/or a tracrRNA extension comprising 10-5000 nucleotides in length, or any combination thereof

Double Guide Nucleic Acid

A nucleic acid-targeting nucleic acid can be a double guide nucleic acid. The double guide nucleic acid can be RNA. The double guide nucleic acid can comprise two separate nucleic acid molecules (i.e. polynucleotides). Each of the two nucleic acid molecules of a double guide nucleic acid-targeting nucleic acid can comprise a stretch of nucleotides that can hybridize to one another such that the complementary nucleotides of the two nucleic acid molecules hybridize to form the double stranded duplex of the protein-binding segment. If not otherwise specified, the term “nucleic acid-targeting nucleic acid” can be inclusive, referring to both single-molecule nucleic acid-targeting nucleic acids and double-molecule nucleic acid-targeting nucleic acids.

A double-guide nucleic acid-targeting nucleic acid can comprise 1) a first nucleic acid molecule comprising a spacer extension sequence from 10-5000 nucleotides in length; a spacer sequence of 12-30 nucleotides in length, wherein the spacer is at least 50% complementary to a target nucleic acid; and a minimum CRISPR repeat comprising at least 60% identity to a crRNA from a prokaryote (e.g., S. pyogenes) or phage over 6 contiguous nucleotides and wherein the minimum CRISPR repeat has a length from 5-30 nucleotides; and 2) a second nucleic acid molecule of the double-guide nucleic acid-targeting nucleic acid can comprise a minimum tracrRNA sequence comprising at least 60% identity to a tracrRNA from a prokaryote (e.g., S. pyogenes) or phage over 6 contiguous nucleotides and wherein the minimum tracrRNA sequence has a length from 5-30 nucleotides; a 3′ tracrRNA that comprises at least 60% identity to a tracrRNA from a bacterium (e.g., S. pyogenes) over 6 contiguous nucleotides and wherein the 3′ tracrRNA comprises a length from 10-20 nucleotides, and comprises a duplexed region; and/or a tracrRNA extension comprising 10-5000 nucleotides in length, or any combination thereof

In some instances, a double-guide nucleic acid-targeting nucleic acid can comprise 1) a first nucleic acid molecule comprising a spacer extension sequence from 10-5000 nucleotides in length; a spacer sequence of 12-30 nucleotides in length, wherein the spacer is at least 50% complementary to a target nucleic acid; a minimum CRISPR repeat comprising at least 60% identity to a crRNA from a prokaryote (e.g., S. pyogenes) or phage over 6 contiguous nucleotides and wherein the minimum CRISPR repeat has a length from 5-30 nucleotides, and at least 3 unpaired nucleotides of a bulge; and 2) a second nucleic acid molecule of the double-guide nucleic acid-targeting nucleic acid can comprise a minimum tracrRNA sequence comprising at least 60% identity to a tracrRNA from a prokaryote (e.g., S. pyogenes) or phage over 6 contiguous nucleotides and wherein the minimum tracrRNA sequence has a length from 5-30 nucleotides and at least 1 unpaired nucleotide of a bulge, wherein the lunpaired nucleotide of the bulge is located in the same bulge as the 3 unpaired nucleotides of the minimum CRISPR repeat; a 3′ tracrRNA that comprises at least 60% identity to a tracrRNA from a prokaryote (e.g., S. pyogenes) or phage over 6 contiguous nucleotides and wherein the 3′ tracrRNA comprises a length from 10-20 nucleotides, and comprises a duplexed region; a nexus that starts from 1-5 nucleotides downstream of the duplex comprising the minimum CRISPR repeat and the minimum tracrRNA, comprises 1-10 nucleotides, comprises a sequence that can hybridize to a protospacer adjacent motif in a target nucleic acid, can form a hairpin, and is located in the 3′ tracrRNA region; and/or a tracrRNA extension comprising 10-5000 nucleotides in length, or any combination thereof

Complex of a Nucleic Acid-Targeting Nucleic Acid and a Site-Directed Polypeptide

A nucleic acid-targeting nucleic acid can interact with a site-directed polypeptide (e.g., a nucleic acid-guided nucleases, Cas9), thereby forming a complex. The nucleic acid-targeting nucleic acid can guide the site-directed polypeptide to a target nucleic acid.

In some embodiments, a nucleic acid-targeting nucleic acid can be engineered such that the complex (e.g., comprising a site-directed polypeptide and a nucleic acid-targeting nucleic acid) can bind outside of the cleavage site of the site-directed polpeptide. In this case, the target nucleic acid may not interact with the complex and the target nucleic acid can be excised (e.g., free from the complex).

In some embodiments, a nucleic acid-targeting nucleic acid can be engineered such that the complex can bind inside of the cleavage site of the site-directed polpeptide. In this case, the target nucleic acid can interact with the complex and the target nucleic acid can be bound (e.g., bound to the complex).

Any nucleic acid-targeting nucleic acid of the disclosure, a site-directed polypeptide of the disclosure, a donor polynucleotide, and/or any nucleic acid or proteinaceous molecule necessary to carry out the embodiments of the methods of the disclosure may be recombinant, purified and/or isolated.

Nucleic Acids Encoding a Nucleic Acid-Targeting Nucleic Acid and/or a Site-Directed Polypeptide

The present disclosure provides for a nucleic acid comprising a nucleotide sequence encoding a nucleic acid-targeting nucleic acid of the disclosure, a site-directed polypeptide of the disclosure, a donor polynucleotide, and/or any nucleic acid or proteinaceous molecule necessary to carry out the embodiments of the methods of the disclosure. In some embodiments, the nucleic acid encoding a nucleic acid-targeting nucleic acid of the disclosure, a site-directed polypeptide of the disclosure, a donor polynucleotide, and/or any nucleic acid or proteinaceous molecule necessary to carry out the embodiments of the methods of the disclosure can be a vector (e.g., a recombinant expression vector).

In some embodiments, the recombinant expression vector can be a viral construct, (e.g., a recombinant adeno-associated virus construct), a recombinant adenoviral construct, a recombinant lentiviral construct, a recombinant retroviral construct, etc.

Suitable expression vectors can include, but are not limited to, viral vectors (e.g. viral vectors based on vaccinia virus, poliovirus, adenovirus, adeno-associated virus, SV40, herpes simplex virus, human immunodeficiency virus, a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus), plant vectors (e.g., T-DNA vector), and the like. The following vectors can be provided by way of non-limiting example, for eukaryotic host cells: pXT1, pSG5, pSVK3, pBPV, pMSG, and pSVLSV40 (Pharmacia). Other vectors may be used so long as they are compatible with the host cell.

In some instances, a vector can be a linearized vector. A linearized vector can comprise a site-directed polypeptide and/or a nucleic acid-targeting nucleic acid. A linearized vector may not be a circular plasmid. A linearized vector can comprise a double-stranded break. A linearized vector may comprise a sequence encoding a fluorescent protein (e.g., orange fluorescent protein (OFP)). A linearized vector may comprise a sequence encoding an antigen (e.g., CD4). A linearized vector can be linearized (e.g., cut) in a region of the vector encoding parts of the nucleic acid-targeting nucleic acid. For example a linearized vector can be linearized (e.g., cut) in a region of the nucleic acid-targeting nucleic acid 5′ to the crRNA portion of the nucleic acid-targeting nucleic acid. A linearized vector can be linearized (e.g., cut) in a region of the nucleic acid-targeting nucleic acid 3′ to the spacer extension sequence of the nucleic acid-targeting nucleic acid. A linearized vector can be linearized (e.g., cut) in a region of the nucleic acid-targeting nucleic acid encoding the crRNA sequence of the nucleic acid-targeting nucleic acid. In some instances, a linearized vector or a closed supercoiled vector comprises a sequence encoding a site-directed polypeptide (e.g., Cas9), a promoter driving expression of the sequence encoding the site-directed polypeptide (e.g., CMV promoter), a sequence encoding a linker (e.g., 2A), a sequence encoding a marker (e.g., CD4 or OFP), a sequence encoding portion of a nucleic acid-targeting nucleic acid, a promoter driving expression of the sequence encoding a portion of the nucleic acid-targeting nucleic acid, and a sequence encoding a selectable marker (e.g., ampicillin), or any combination thereof.

A vector can comprise a transcription and/or translation control element. Depending on the host/vector system utilized, any of a number of suitable transcription and translation control elements, including constitutive and inducible promoters, transcription enhancer elements, transcription terminators, etc. may be used in the expression vector.

In some embodiments, a nucleotide sequence encoding a nucleic acid-targeting nucleic acid of the disclosure, a site-directed polypeptide of the disclosure, a donor polynucleotide, and/or any nucleic acid or proteinaceous molecule necessary to carry out the embodiments of the methods of the disclosure can be operably linked to a control element (e.g., a transcriptional control element), such as a promoter. The transcriptional control element may be functional in a eukaryotic cell, (e.g., a mammalian cell), a prokaryotic cell (e.g., bacterial or archaeal cell). In some embodiments, a nucleotide sequence encoding a nucleic acid-targeting nucleic acid of the disclosure, a site-directed polypeptide of the disclosure, a donor polynucleotide, and/or any nucleic acid or proteinaceous molecule necessary to carry out the embodiments of the methods of the disclosure can be operably linked to multiple control elements. Operable linkage to multiple control elements can allow expression of the nucleotide sequence encoding a nucleic acid-targeting nucleic acid of the disclosure, a site-directed polypeptide of the disclosure, a donor polynucleotide, and/or any nucleic acid or proteinaceous molecule necessary to carry out the embodiments of the methods of the disclosure in either prokaryotic or eukaryotic cells.

Non-limiting examples of suitable eukaryotic promoters (i.e. promoters functional in a eukaryotic cell) can include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-active promoter (CAG), murine stem cell virus promoter (MSCV), phosphoglycerate kinase-1 locus promoter (PGK) and mouse metallothionein-I. The promoter can be a fungi promoter. The promoter can be a plant promoter. A database of plant promoteres can be found (e.g., PlantProm). The expression vector may also contain a ribosome binding site for translation initiation and a transcription terminator. The expression vector may also include appropriate sequences for amplifying expression. The expression vector may also include nucleotide sequences encoding non-native tags (e.g., 6×His tag, hemagglutinin tag, green fluorescent protein, etc.) that are fused to the site-directed polypeptide, thus resulting in a fusion protein.

In some embodiments, a nucleotide sequence or sequences encoding a nucleic acid-targeting nucleic acid of the disclosure, a site-directed polypeptide of the disclosure, a donor polynucleotide, and/or any nucleic acid or proteinaceous molecule necessary to carry out the embodiments of the methods of the disclosure can be operably linked to an inducible promoter (e.g., heat shock promoter, tetracycline-regulated promoter, steroid-regulated promoter, metal-regulated promoter, estrogen receptor-regulated promoter, etc.). In some embodiments, a nucleotide sequence encoding a nucleic acid-targeting nucleic acid of the disclosure, a site-directed polypeptide of the disclosure, a donor polynucleotide, and/or any nucleic acid or proteinaceous molecule necessary to carry out the embodiments of the methods of the disclosure can be operably linked to a constitutive promoter (e.g., CMV promoter, UBC promoter). In some embodiments, the nucleotide sequence can be operably linked to a spatially restricted and/or temporally restricted promoter (e.g., a tissue specific promoter, a cell type specific promoter, etc.).

A nucleotide sequence or sequences encoding a nucleic acid-targeting nucleic acid of the disclosure, a site-directed polypeptide of the disclosure, a donor polynucleotide, and/or any nucleic acid or proteinaceous molecule necessary to carry out the embodiments of the methods of the disclosure can be packaged into or on the surface of biological compartments for delivery to cells. Biological compartments can include, but are not limited to, viruses (lentivirus, adenovirus), nanospheres, liposomes, quantum dots, nanoparticles, polyethylene glycol particles, hydrogels, and micelles.

Introduction of the complexes, polypeptides, and nucleic acids of the disclosure into cells can occur by viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Transgenic Cells and Organisms

The disclosure provides for transgenic cells and organisms generated or modified using methods and compositions of the disclosure. The nucleic acid of a genetically modified host cell and/or transgenic organism can be targeted for genome engineering.

Exemplary cells that can be used to generate transgenic cells according to the methods of the disclosure can include, but are not limited to, HeLa cell, Chinese Hamster Ovary cell, 293-T cell, a pheochromocytoma, a neuroblastomas fibroblast, a rhabdomyosarcoma, a dorsal root ganglion cell, a NSO cell, Tobacco BY-2, CV-I (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C 1271 (ATCC CRL 1616), BS-C-1 (ATCC, CCL 26), MRC-5 (ATCC CCL 171), L-cells, HEK-293 (ATCC CRL1 573) and PC 12 (ATCC CRL-1721), HEK293T (ATCC CRL-11268), (ATCC CRL-1378), SH-SY5Y (ATCC CRL-2266), MDCK (ATCC CCL-34), RH30 (ATCC CRL-2061), HepG2 (ATCC HB-8065), ND7/23 (ECACC 92090903), CHO (ECACC 85050302), Vera (ATCC CCL 81), Caco-2 (ATCC HTB 37), K562 (ATCC CCL 243), Jurkat (ATCC TIB-152), Per.Có, Huvec (ATCC Human Primary PCS 100-010, Mouse CRL 2514, CRL 2515, CRL 2516), HuH-1-7D12 (ECACC 01042712), 293 (ATCC CRL 10852), A549 (ATCC CCL 185), IMR-90 (ATCC CCL 186), MCF-7 (ATC HTB-22), U-2 OS (ATCC HTB-96), and T84 (ATCC CCL 248), or any cell available at American Type Culture Collection (ATCC), or any combination thereof.

Organisms that can be transgenic can include bacteria, archaea, single-cell eukaryotes, plants, algae, fungi (e.g., yeast), invertebrates (e.g., fruit fly, cnidarian, echinoderm, nematode, etc), vertebrates (e.g., fish, amphibian, reptile, bird, mammal), mammals (e.g., a pig, a cow, a goat, a sheep, a rodent, a rat, a mouse, a non-human primate, a human, etc.), etc.

Transgenic organisms can comprise genetically modified cells. Transgenic organisms and/or genetically modified cells can comprise organisms and/or cells that have been genetically modified with an exogenous nucleic acid comprising a nucleotide sequence encoding nucleic acid-targeting nucleic acid of the disclosure, an effector protein, and/or a site-directed polypeptide, or any combination thereof.

A genetically modified cell can comprise an exogenous site-directed polypeptide and/or an exogenous nucleic acid comprising a nucleotide sequence encoding a site-directed polypeptide. Expression of the site-directed polypeptide in the cell may take 0.1, 0.2, 0.5, 1, 2, 3, 4, 5, 6, or more days. Cells, introduced with the site-directed polypeptide, may be grown for 0.1, 0.2, 0.5, 1, 2, 3, 4, 5, 6, 7, 8, 9, 10 or even more days before the cells can be removed from cell culture and/or host organism.

Subjects

The disclosure provides for performing the methods of the disclosure in a subject. A subject can be a human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician). A subject can be a plant or a crop.

Kits

The present disclosure provides kits for carrying out the methods of the disclosure. A kit can include one or more of: A nucleic acid-targeting nucleic acid of the disclosure, a polynucleotide encoding a nucleic acid-targeting nucleic acid, a site-directed polypeptide of the disclosure, a polynucleotide encoding a site-directed polypeptide, a donor polynucleotide, and/or any nucleic acid or proteinaceous molecule necessary to carry out the embodiments of the methods of the disclosure, or any combination thereof

A nucleic acid-targeting nucleic acid of the disclosure, a polynucleotide encoding a nucleic acid-targeting nucleic acid, a site-directed polypeptide of the disclosure, a polynucleotide encoding a site-directed polypeptide, a donor polynucleotide, and/or any nucleic acid or proteinaceous molecule necessary to carry out the embodiments of the methods of the disclosure are described in detail above.

A kit can comprise: (1) a vector comprising a nucleotide sequence encoding a nucleic acid-targeting nucleic acid of the disclosure, and (2) a vector comprising a nucleotide sequence encoding the site-directed polypeptide of the disclosure and (2) a reagent for reconstitution and/or dilution of the vectors.

A kit can comprise: (1) a vector comprising (i) a nucleotide sequence encoding a nucleic acid-targeting nucleic acid of the disclosure, and (ii) a nucleotide sequence encoding the site-directed polypeptide of the disclosure and (2) a reagent for reconstitution and/or dilution of the vector.

A kit can comprise: (1) a vector comprising a nucleotide sequence encoding a nucleic acid-targeting nucleic acid of the disclosure, (2) a vector comprising a nucleotide sequence encoding the site-directed polypeptide of the disclosure, (3) a vector comprising a nucleotide sequence encoding a donor polynucleotide, and/or any nucleic acid or proteinaceous molecule necessary to carry out the embodiments of the methods of the disclosure, and (4) a reagent for reconstitution and/or dilution of the vectors.

A kit can comprise: (1) a vector comprising (i) a nucleotide sequence encoding a nucleic acid-targeting nucleic acid of the disclosure, (ii) a nucleotide sequence encoding the site-directed polypeptide of the disclosure, (2) a vector comprising a nucleotide sequence encoding a donor polynucleotide, and/or any nucleic acid or proteinaceous molecule necessary to carry out the embodiments of the methods of the disclosure, and (3) a reagent for reconstitution and/or dilution of the recombinant expression vectors.

In some embodiments of any of the above kits, the kit can comprise a single guide nucleic acid-targeting nucleic acid. In some embodiments of any of the above kits, the kit can comprise a double guide nucleic acid-targeting nucleic acid. In some embodiments of any of the above kits, the kit can comprise two or more double guide or single guide nucleic acid-targeting nucleic acids. In some embodiments, a vector may encode for a nucleic acid targeting nucleic acid.

In some embodiments of any of the above kits, the kit can further comprise a donor polynucleotide, or a polynucleotide sequence encoding the donor polynucleotide, to effect the desired genetic modification. Components of a kit can be in separate containers; or can be combined in a single container.

A kit described above further comprise one or more additional reagents, where such additional reagents can be selected from: a buffer, a buffer for introducing the a polypeptide or polynucleotide item of the kit into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, or a diluting buffer.

In some instances, a kit can comprise one or more additional reagents specific for plants and/or fungi. One or more additional reagents for plants and/or fungi can include, for example, soil, nutrients, plants, seeds, spores, Agrobacterium, T-DNA vector, and a pBINAR vector.

In addition to above-mentioned components, a kit can further include instructions for using the components of the kit to practice the methods. The instructions for practicing the methods are generally recorded on a suitable recording medium. For example, the instructions may be printed on a substrate, such as paper or plastic, etc. The instructions may be present in the kits as a package insert, in the labeling of the container of the kit or components thereof (i.e., associated with the packaging or subpackaging) etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this embodiment is a kit that includes a web address where the instructions can be viewed and/or from which the instructions can be downloaded. As with the instructions, this means for obtaining the instructions can be recorded on a suitable substrate.

Pharmaceutical Compositions

Molecules, such as a nucleic acid-targeting nucleic acid of the disclosure as described herein, a polynucleotide encoding a nucleic acid-targeting nucleic acid, a site-directed polypeptide of the disclosure, a polynucleotide encoding a site-directed polypeptide, a donor polynucleotide, and/or any nucleic acid or proteinaceous molecule necessary to carry out the embodiments of the methods of the disclosure, can be formulated in a pharmaceutical composition.

A pharmaceutical composition can comprise a combination of any molecules described herein with other chemical components, such as carriers, stabilizers, diluents, dispersing agents, suspending agents, thickening agents, and/or excipients. The pharmaceutical composition can facilitate administration of the molecule to an organism. Pharmaceutical compositions can be administered in therapeutically-effective amounts as pharmaceutical compositions by various forms and routes including, for example, intravenous, subcutaneous, intramuscular, oral, rectal, aerosol, parenteral, ophthalmic, pulmonary, transdermal, vaginal, otic, nasal, and topical administration.

A pharmaceutical composition can be administered in a local or systemic manner, for example, via injection of the molecule directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated readily by combining the molecules with pharmaceutically-acceptable carriers or excipients. Such carriers can be used to formulate tablets, powders, pills, dragees, capsules, liquids, gels, syrups, elixirs, slurries, suspensions and the like, for oral ingestion by a subject.

Pharmaceutical preparations for oral use can be obtained by mixing one or more solid excipient with one or more of the molecules described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used, which can contain an excipient such as gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations which can be used orally can include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. A gelatin can be alkaline-processed. The push-fit capsules can comprise the active ingredients in admixture with filler such as lactose, binders such as starches, and/or lubricants such as talc or magnesium stearate and, stabilizers. In soft capsules, the molecule can be dissolved or suspended in suitable liquids, such as fatty oils, liquid paraffin, or liquid polyethylene glycols. Stabilizers can be added. All formulations for oral administration are provided in dosages suitable for such administration.

For buccal or sublingual administration, the compositions can be tablets, lozenges, or gels.

Parental injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration can include aqueous solutions of the active compounds in water-soluble form.

Suspensions of molecules can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the molecules to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can comprise solubilizers, stabilizers, tonicity enhancing agents, buffers and preservatives.

Formulations suitable for transdermal administration of the molecules can employ transdermal delivery devices and transdermal delivery patches, and can be lipophilic emulsions or buffered aqueous solutions, dissolved and/or dispersed in a polymer or an adhesive. Such patches can be constructed for continuous, pulsatile, or on demand delivery of molecules. Transdermal delivery can be accomplished by means of iontophoretic patches and the like. Additionally, transdermal patches can provide controlled delivery. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin.

For administration by inhalation, the molecule can be in a form as an aerosol, a mist, or a powder. Pharmaceutical compositions can be delivered in the form of an aerosol spray presentation from pressurized packs or a nebuliser, with the use of a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compounds and a suitable powder base such as lactose or starch.

The molecules can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone and PEG. In suppository forms of the compositions, a low-melting wax such as a mixture of fatty acid glycerides or cocoa butter can be used.

In practicing the methods of the disclosure, therapeutically-effective amounts of the compounds described herein can be administered in pharmaceutical compositions to a subject having a disease or condition to be treated. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the molecule into preparations that can be used pharmaceutically. Formulation can be modified depending upon the route of administration chosen. Pharmaceutical compositions comprising a molecule described herein can be manufactured, for example, by mixing, dissolving, granulating, dragee-making, levigating, emulsifying, encapsulating, entrapping, or compression processes.

The pharmaceutical compositions can include at least one pharmaceutically acceptable carrier, diluent, or excipient and molecule described herein as free-base or pharmaceutically-acceptable salt form. The methods and pharmaceutical compositions described herein include the use crystalline forms (also known as polymorphs), and active metabolites of these compounds having the same type of activity.

Methods for the preparation of compositions comprising the compounds described herein can include formulating the molecule with one or more inert, pharmaceutically-acceptable excipients or carriers to form a solid, semi-solid, or liquid composition. Solid compositions can include, for example, powders, tablets, dispersible granules, capsules, cachets, and suppositories. Liquid compositions can include, for example, solutions in which a compound is dissolved, emulsions comprising a compound, or a solution containing liposomes, micelles, or nanoparticles comprising a compound as disclosed herein. Semi-solid compositions can include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms can include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, and nutraceuticals, or any combination thereof.

Non-limiting examples of pharmaceutically-acceptable excipients can include granulating agents, binding agents, lubricating agents, disintegrating agents, sweetening agents, glidants, anti-adherents, anti-static agents, surfactants, anti-oxidants, gums, coating agents, coloring agents, flavouring agents, coating agents, plasticizers, preservatives, suspending agents, emulsifying agents, plant cellulosic material, and spheronization agents, or any combination thereof

A composition can be, for example, an immediate release form or a controlled release formulation. An immediate release formulation can be formulated to allow the molecules to act rapidly. Non-limiting examples of immediate release formulations can include readily dissolvable formulations. A controlled release formulation can be a pharmaceutical formulation that has been adapted such that drug release rates and drug release profiles can be matched to physiological and chronotherapeutic requirements or, alternatively, has been formulated to effect release of a drug at a programmed rate. Non-limiting examples of controlled release formulations can include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, granular masses, and the like.

A controlled release formulation can be a delayed release form. A delayed release form can be formulated to delay a molecule's action for an extended period of time. A delayed release form can be formulated to delay the release of an effective dose of one or more molecules, for example, for about 4, about 8, about 12, about 16, or about 24 hours.

A controlled release formulation can be a sustained release form. A sustained release form can be formulated to sustain, for example, the molecule's action over an extended period of time. A sustained release form can be formulated to provide an effective dose of any molecule described herein (e.g., provide a physiologically-effective blood profile) over about 4, about 8, about 12, about 16 or about 24 hours.

Methods of Administration and Treatment Methods.

Pharmaceutical compositions containing molecules described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Amounts effective for this use can vary based on the severity and course of the disease or condition, previous therapy, the subject's health status, weight, and response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills. The molecules can be packed together or separately, in a single package or in a plurality of packages. One or all of the therapeutic agents can be given in multiple doses. If not simultaneous, the timing between the multiple doses may vary to as much as about a month.

Molecules described herein can be administered before, during, or after the occurrence of a disease or condition, and the timing of administering the composition containing a compound can vary. For example, the pharmaceutical compositions can be used as a prophylactic and can be administered continuously to subjects with a propensity to conditions or diseases in order to prevent the occurrence of the disease or condition. The molecules and pharmaceutical compositions can be administered to a subject during or as soon as possible after the onset of the symptoms. The administration of the molecules can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A molecule can be administered as soon as is practicable after the onset of a disease or condition is detected or suspected, and for a length of time necessary for the treatment of the disease, such as, for example, from about 1 month to about 3 months. The length of treatment can vary for each subject.

A molecule can be packaged into a biological compartment. A biological compartment comprising the molecule can be administered to a subject. Biological compartments can include, but are not limited to, viruses (lentivirus, adenovirus), nanospheres, liposomes, quantum dots, nanoparticles, microparticles, nanocapsules, vesicles, polyethylene glycol particles, hydrogels, and micelles.

For example, a biological compartment can comprise a liposome. A liposome can be a self-assembling structure comprising one or more lipid bilayers, each of which can comprise two monolayers containing oppositely oriented amphipathic lipid molecules. Amphipathic lipids can comprise a polar (hydrophilic) headgroup covalently linked to one or two or more non-polar (hydrophobic) acyl or alkyl chains. Energetically unfavorable contacts between the hydrophobic acyl chains and a surrounding aqueous medium induce amphipathic lipid molecules to arrange themselves such that polar headgroups can be oriented towards the bilayer's surface and acyl chains are oriented towards the interior of the bilayer, effectively shielding the acyl chains from contact with the aqueous environment.

Examples of preferred amphipathic compounds used in liposomes can include phosphoglycerides and sphingolipids, representative examples of which include phosphatidylcholine, phosphatidylethanolamine, phosphatidylserine, phosphatidylinositol, phosphatidic acid, phoasphatidylglycerol, palmitoyloleoyl phosphatidylcholine, lysophosphatidylcholine, lysophosphatidylethanolamine, dimyristoylphosphatidylcholine (DMPC), dipalmitoylphosphatidylcholine (DPPC), dioleoylphosphatidylcholine, distearoylphosphatidylcholine (DSPC), dilinoleoylphosphatidylcholine and egg sphingomyelin, or any combination thereof

A biological compartment can comprise a nanoparticle. A nanoparticle can comprise a diameter of from about 40 nanometers to about 1.5 micrometers, from about 50 nanometers to about 1.2 micrometers, from about 60 nanometers to about 1 micrometer, from about 70 nanometers to about 800 nanometers, from about 80 nanometers to about 600 nanometers, from about 90 nanometers to about 400 nanometers, from about 100 nanometers to about 200 nanometers.

In some instances, as the size of the nanoparticle increases, the release rate can be slowed or prolonged and as the size of the nanoparticle decreases, the release rate can be increased.

The amount of albumin in the nanoparticles can range from about 5% to about 85% albumin (v/v), from about 10% to about 80%, from about 15% to about 80%, from about 20% to about 70% albumin (v/v), from about 25% to about 60%, from about 30% to about 50%, or from about 35% to about 40%. The pharmaceutical composition can comprise up to 30, 40, 50, 60, 70 or 80% or more of the nanoparticle. In some instances, the nucleic acid molecules of the disclosure can be bound to the surface of the nanoparticle.

A biological compartment can comprise a virus. The virus can be a delivery system for the pharmaceutical compositions of the disclosure. Exemplary viruses can include lentivirus, retrovirus, adenovirus, herpes simplex virus I or II, parvovirus, reticuloendotheliosis virus, and adeno-associated virus (AAV). Pharmaceutical compositions of the disclosure can be delivered to a cell using a virus. The virus can infect and transduce the cell in vivo, ex vivo, or in vitro. In ex vivo and in vitro delivery, the transduced cells can be administered to a subject in need of therapy.

Pharmaceutical compositions can be packaged into viral delivery systems. For example, the compositions can be packaged into virions by a HSV-1 helper virus-free packaging system.

Viral delivery systems (e.g., viruses comprising the pharmaceutical compositions of the disclosure) can be administered by direct injection, stereotaxic injection, intracerebroventricularly, by minipump infusion systems, by convection, catheters, intravenous, parenteral, intraperitoneal, and/or subcutaenous injection, to a cell, tissue, or organ of a subject in need. In some instances, cells can be transduced in vitro or ex vivo with viral delivery systems. The transduced cells can be administered to a subject having a disease. For example, a stem cell can be transduced with a viral delivery system comprising a pharmaceutical composition and the stem cell can be implanted in the patient to treat a disease. In some instances, the dose of transduced cells given to a subject can be about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single dose.

Pharmaceutical compositions in biological compartments can be used to treatinflammatory diseases such as arthritis, cancers, such as, for example, bone cancer, breast cancer, skin cancer, prostate cancer, liver cancer, lung cancer, throat cancer and kidney cancer, bacterial infections, to treat nerve damage, lung, liver and kidney diseases, eye treatment, spinal cord injuries, heart disease, arterial disease.

Introduction of the biological compartments into cells can occur by viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Dosage

Pharmaceutical compositions described herein can be in unit dosage forms suitable for single administration of precise dosages. In unit dosage form, the formulation can be divided into unit doses containing appropriate quantities of one or more compounds. The unit dosage can be in the form of a package containing discrete quantities of the formulation. Non-limiting examples can include packaged tablets or capsules, and powders in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A molecule described herein can be present in a composition in a range of from about 1 mg to about 2000 mg; from about 5 mg to about 1000 mg, from about 10 mg to about 25 mg to 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A molecule described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

A molecule (e.g., site-directed polypeptide, nucleic acid-targeting nucleic acid and/or complex of a site-directed polypeptide and a nucleic acid-targeting nucleic acid) described herein can be present in a composition that provides at least 0.1, 0.5, 1, 1.5, 2, 2.5, 3, 3.5, 4, 4.5, 5, 5.5, 6, 6.5, 10 or more units of activity/mg molecule. In some embodiments, the total number of units of activity of the molecule delivered to a subject is at least 25,000, 30,000, 35,000, 40,000, 45,000, 50,000, 60,000, 70,000, 80,000, 90,000, 110,000, 120,000, 130,000, 140,000, 150,000, 160,000, 170,000, 180,000, 190,000, 200,000, 210,000, 220,000, 230,000, or 250,000 or more units. In some embodiments, the total number of units of activity of the molecule delivered to a subject is at most 25,000, 30,000, 35,000, 40,000, 45,000, 50,000, 60,000, 70,000, 80,000, 90,000, 110,000, 120,000, 130,000, 140,000, 150,000, 160,000, 170,000, 180,000, 190,000, 200,000, 210,000, 220,000, 230,000, or 250,000 or more units.

In some embodiments, at least about 10,000 units of activity is delivered to a subject, normalized per 50 kg body weight. In some embodiments, at least about 10,000, 15,000, 25,000, 30,000, 35,000, 40,000, 45,000, 50,000, 60,000, 70,000, 80,000, 90,000, 110,000, 120,000, 130,000, 140,000, 150,000, 160,000, 170,000, 180,000, 190,000, 200,000, 210,000, 220,000, 230,000, or 250,000 units or more of activity of the molecule is delivered to the subject, normalized per 50 kg body weight. In some embodiments, a therapeutically effective dose comprises at least 5×10⁵, 1×10⁶, 2×10⁶, 3×10⁶, 4, 10⁶, 5×10⁶, 6×10⁶, 7×10⁶, 8×10⁶, 9×10⁶, 1×10⁷, 1.1×10⁷, 1.2×10⁷, 1.5×10⁷, 1.6×10⁷, 1.7×10⁷, 1.8×10⁷, 1.9×10⁷, 2×10⁷, 2.1×10⁷, or 3×10⁷ or more units of activity of the molecule. In some embodiments, a therapeutically effective dose comprises at most 5×10⁵ 1×10⁶, 2×10⁶, 3×10⁶, 4, 10⁶, 5×10⁶, 6×10⁶, 7×10⁶, 8×10⁶, 9×10⁶, 1×10⁷, 1.1×10⁷, 1.2×10⁷, 1.5×10⁷, 1.6×10⁷, 1.7×10⁷, 1.8×10⁷, 1.9×10⁷, 2×10⁷, 2.1×10⁷, or 3×10⁷ or more units of activity of the molecule.

In some embodiments, a therapeutically effective dose is at least about 10,000, 15,000, 20,000, 22,000, 24,000, 25,000, 30,000, 40,000, 50,000, 60,000, 70,000, 80,000, 90,000, 100,000, 125,000, 150,000, 200,000, or 500,000 units/kg body weight. In some embodiments, a therapeutically effective dose is at most about 10,000, 15,000, 20,000, 22,000, 24,000, 25,000, 30,000, 40,000, 50,000, 60,000, 70,000, 80,000, 90,000, 100,000, 125,000, 150,000, 200,000, or 500,000 units/kg body weight.

In some embodiments, the activity of the molecule delivered to a subject is at least 10,000, 11,000, 12,000, 13,000, 14,000, 20,000, 21,000, 22,000, 23,000, 24,000, 25,000, 26,000, 27,000, 28,000, 30,000, 32,000, 34,000, 35,000, 36,000, 37,000, 40,000, 45,000, or 50,000 or more U/mg of molecule. In some embodiments, the activity of the molecule delivered to a subject is at most 10,000, 11,000, 12,000, 13,000, 14,000, 20,000, 21,000, 22,000, 23,000, 24,000, 25,000, 26,000, 27,000, 28,000, 30,000, 32,000, 34,000, 35,000, 36,000, 37,000, 40,000, 45,000, or 50,000 or more U/mg of molecule.

Pharmacokinetic and Pharmacodynamic Measurements

Pharmacokinetic and pharmacodynamic data can be obtained by various experimental techniques. Appropriate pharmacokinetic and pharmacodynamic profile components describing a particular composition can vary due to variations in drug metabolism in human subjects. Pharmacokinetic and pharmacodynamic profiles can be based on the determination of the mean parameters of a group of subjects. The group of subjects includes any reasonable number of subjects suitable for determining a representative mean, for example, 5 subjects, 10 subjects, 15 subjects, 20 subjects, 25 subjects, 30 subjects, 35 subjects, or more. The mean can be determined by calculating the average of all subject's measurements for each parameter measured. A dose can be modulated to achieve a desired pharmacokinetic or pharmacodynamics profile, such as a desired or effective blood profile, as described herein.

The pharmacokinetic parameters can be any parameters suitable for describing a molecule. For example, the Cₘₐₓ can be, for example, not less than about 25 ng/mL; not less than about 50 ng/mL; not less than about 75 ng/mL; not less than about 100 ng/mL; not less than about 200 ng/mL; not less than about 300 ng/mL; not less than about 400 ng/mL; not less than about 500 ng/mL; not less than about 600 ng/mL; not less than about 700 ng/mL; not less than about 800 ng/mL; not less than about 900 ng/mL; not less than about 1000 ng/mL; not less than about 1250 ng/mL; not less than about 1500 ng/mL; not less than about 1750 ng/mL; not less than about 2000 ng/mL; or any other Cₘₐₓ appropriate for describing a pharmacokinetic profile of a molecule described herein.

The Tₘₐₓ of a molecule described herein can be, for example, not greater than about 0.5 hours, not greater than about 1 hours, not greater than about 1.5 hours, not greater than about 2 hours, not greater than about 2.5 hours, not greater than about 3 hours, not greater than about 3.5 hours, not greater than about 4 hours, not greater than about 4.5 hours, not greater than about 5 hours, or any other Tₘₐₓ appropriate for describing a pharmacokinetic profile of a molecule described herein.

The AUC_{(0-inf)} of a molecule described herein can be, for example, not less than about 50 ng·hr/mL, not less than about 100 ng/hr/mL, not less than about 150 ng/hr/mL, not less than about 200 ng·hr/mL, not less than about 250 ng/hr/mL, not less than about 300 ng/hr/mL, not less than about 350 ng/hr/mL, not less than about 400 ng/hr/mL, not less than about 450 ng/hr/mL, not less than about 500 ng/hr/mL, not less than about 600 ng/hr/mL, not less than about 700 ng/hr/mL, not less than about 800 ng/hr/mL, not less than about 900 ng/hr/mL, not less than about 1000 ng·hr/mL, not less than about 1250 ng/hr/mL, not less than about 1500 ng/hr/mL, not less than about 1750 ng/hr/mL, not less than about 2000 ng/hr/mL, not less than about 2500 ng/hr/mL, not less than about 3000 ng/hr/mL, not less than about 3500 ng/hr/mL, not less than about 4000 ng/hr/mL, not less than about 5000 ng/hr/mL, not less than about 6000 ng/hr/mL, not less than about 7000 ng/hr/mL, not less than about 8000 ng/hr/mL, not less than about 9000 ng/hr/mL, not less than about 10,000 ng/hr/mL, or any other AUC_{(0-inf)} appropriate for describing a pharmacokinetic profile of a molecule described herein.

The plasma concentration of a molecule described herein about one hour after administration can be, for example, not less than about 25 ng/mL, not less than about 50 ng/mL, not less than about 75 ng/mL, not less than about 100 ng/mL, not less than about 150 ng/mL, not less than about 200 ng/mL, not less than about 300 ng/mL, not less than about 400 ng/mL, not less than about 500 ng/mL, not less than about 600 ng/mL, not less than about 700 ng/mL, not less than about 800 ng/mL, not less than about 900 ng/mL, not less than about 1000 ng/mL, not less than about 1200 ng/mL, or any other plasma concentration of a molecule described herein.

The pharmacodynamic parameters can be any parameters suitable for describing pharmaceutical compositions of the disclosure. For example, the pharmacodynamic profile can exhibit decreases in factors associated with inflammation after, for example, about 2 hours, about 4 hours, about 8 hours, about 12 hours, or about 24 hours.

Pharmaceutically-Acceptable Salts

The disclosure provides the use of pharmaceutically-acceptable salts of any molecule described herein. Pharmaceutically-acceptable salts can include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt, or any combination thereof

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine, or any combination thereof.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt, or any combination thereof

Acid addition salts can arise from the addition of an acid to a molecule of the disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid, or any combination thereof

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt, or any combination thereof

3′ Engineered Nucleic Acid Targeting Nucleic Acids

The nucleic acid-targeting nucleic acids of the disclosure can be modified to delete the 3′ hairpin region. At least 5, 10, 15, 20, 25, 30, 35, 40, 45, 50, 55, 60, 65, 70, 75, 80, 85, 90, 95, or 100% of the 3′ hairpin regions can be deleted from the nucleic acid-targeting nucleic acid. At most 5, 10, 15, 20, 25, 30, 35, 40, 45, 50, 55, 60, 65, 70, 75, 80, 85, 90, 95, or 100% of the 3′ hairpin regions can be deleted from the nucleic acid-targeting nucleic acid. The first hairpin can be deleted from the nucleic acid-targeting nucleic acid. The second hairpin can be deleted from the nucleic acid-targeting nucleic acid. Both the first and second 3′ hairpins can be deleted from the nucleic acid-targeting nucleic acid from the nucleic acid-targeting nucleic acid.

A nucleic acid-targeting nucleic acid can be chemically synthesized. A nucleic acid-targeting nucleic acid with a deletion in the 3′ end can be chemically synthesized. Oligonucleotide synthesis can occur with solid phase chemistry (e.g., the phosphoramidite method). For example, a phosphoramidite can be reacted with a support-bound nucleotide, or oligonucleotide, in the presence of an activator. The phosphoroamidite coupling-product can be oxidized to afford a protected phosphate. An example of a phosphoramidite derivative is 1H-tetrazole. In some instances, oligonucleotide synthesis is performed in solution.

A 3′ engineered nucleic acid-targeting nucleic acid can bind a target nucleic acid with a greater or lesser binding constant than a wild-type nucleic acid-targeting nucleic acid (e.g., without a 3′ engineered deletion). A 3′ engineered nucleic acid-targeting nucleic acid can bind a target nucleic acid with at least 1-fold, 2-fold, 3-fold, 4-fold, 5-fold, 6-fold, 7-fold, 8-fold, 9-fold, or 10-fold or more greater or lesser binding affinity than a nucleic acid-targeting nucleic acid without a 3′ engineered deletion. A 3′ engineered nucleic acid-targeting nucleic acid can bind a target nucleic acid with at most 1-fold, 2-fold, 3-fold, 4-fold, 5-fold, 6-fold, 7-fold, 8-fold, 9-fold, or 10-fold or more greater or lesser binding affinity than a nucleic acid-targeting nucleic acid without a 3′ engineered deletion.

A 3′ engineered nucleic acid-targeting nucleic acid can reduce off-target binding compared to a nucleic acid-targeting nucleic acid without a 3′ engineered deletion. A 3′ engineered nucleic acid-targeting nucleic acid can reduce off-targeting binding by at least 1-fold, 2-fold, 3-fold, 4-fold, 5-fold, 6-fold, 7-fold, 8-fold, 9-fold, or 10-fold or more than an un-engineered nucleic acid-targeting nucleic acid with a 3′ engineered deletion. A 3′ engineered nucleic acid-targeting nucleic acid can reduce off-targeting binding by at most 1-fold, 2-fold, 3-fold, 4-fold, 5-fold, 6-fold, 7-fold, 8-fold, 9-fold, or 10-fold or more than an un-engineered nucleic acid-targeting nucleic acid with a 3′ engineered deletion.

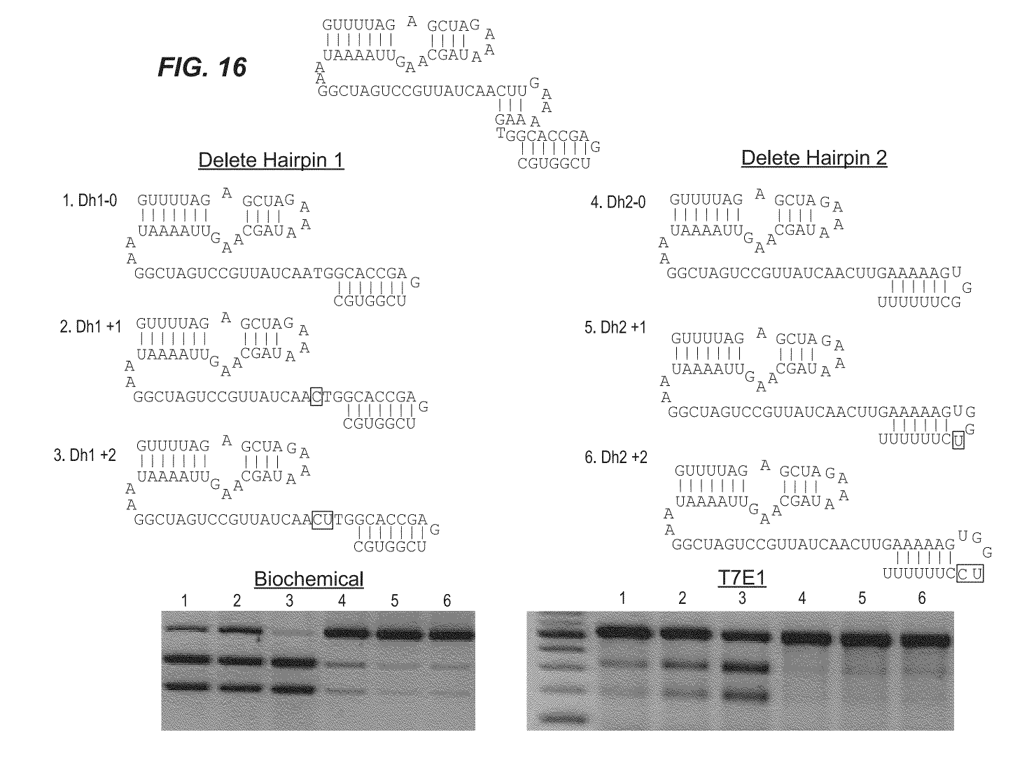

FIG. 16 illustrates the activity of nucleic acid-targeting nucleic acids with either the first or second hairpin of the 3′ tracrRNA sequence deleted. The activity assays were performed in an in vitro (biochemical) setting, or in vivo (cell-based) (T7E1).

Nucleic Acid-Targeting Nucleic Acids with an Engineered Loop and Nexus

The disclosure provides for nucleic acid-targeting nucleic acids with modifications to the loop and/or nexus region. An engineered nucleic acid-targeting nucleic acid can comprise a non-natural spacer and a natural nexus. An engineered nucleic acid-targeting nucleic acid can comprise a non-natural spacer and a non-natural nexus. An engineered nucleic acid-targeting nucleic acid can comprise a natural spacer and a non-natural nexus.

An engineered nexus can comprise at least 1, 2, 3, 4, 5, 6, 7, 8, 9, or 10 or more mutations. An engineered nexus can comprise at most 1, 2, 3, 4, 5, 6, 7, 8, 9, or 10 or more mutations. An engineered nexus can comprise at least 1, 2, 3, 4, 5, 6, 7, 8, 9, or 10 or more additional nucleotides inserted into the nexus. An engineered nexus can comprise at most 1, 2, 3, 4, 5, 6, 7, 8, 9, or 10 or more additional nucleotides inserted into the nexus. An engineered nexus can comprise at least 1, 2, 3, 4, 5, 6, 7, 8, 9, or 10 or more additional nucleotides deleted from the nexus. An engineered nexus can comprise at most 1, 2, 3, 4, 5, 6, 7, 8, 9, or 10 or more additional nucleotides deleted from the nexus.

An engineered nexus can comprise an engineered hairpin duplex. The engineered hairpin duplex can comprise at least 1, 2, 3, 4, 5, or more stacked base-paired nucleotides of the duplex. The engineered hairpin duplex can comprise at most 1, 2, 3, 4, 5, or more stacked base-paired nucleotides of the duplex.

An engineered nexus can comprise an engineered loop of the nexus. The engineered loop of the nexus can comprise at least 1, 2, 3, 4, 5, 6, 7, 8, 9, or 10 or more additional nucleotides. The engineered loop of the nexus can comprise at most 1, 2, 3, 4, 5, 6, 7, 8, 9, or 10 or more additional nucleotides.

An engineered loop can comprise at least 1, 2, 3, 4, 5, 6, 7, 8, 9, or 10 or more mutations. An engineered loop can comprise at most 1, 2, 3, 4, 5, 6, 7, 8, 9, or 10 or more mutations. An engineered loop can comprise at least 1, 2, 3, 4, 5, 6, 7, 8, 9, or 10 or more additional nucleotides inserted into the loop. An engineered nexus can comprise at most 1, 2, 3, 4, 5, 6, 7, 8, 9, or 10 or more additional nucleotides inserted into the v. An engineered loop can comprise at least 1, 2, 3, 4, 5, 6, 7, 8, 9, or 10 or more additional nucleotides deleted from the loop. An engineered loop can comprise at most 1, 2, 3, 4, 5, 6, 7, 8, 9, or 10 or more additional nucleotides deleted from the loop.

Generation of Libraries of Nucleic Acid-Targeting Nucleic Acids

In some embodiments, a library of nucleic acid-targeting nucleic acids can be generated which comprise different engineered backbones of nucleic acid-targeting nucleic acids. For example, a library of nucleic acid-targeting nucleic acids can comprise mutated nucleic acid-targeting nucleic acids in which each mutated nucleic acid-targeting nucleic acid can comprise a different mutation. The mutation can comprise at least 1, 2, 3, 4, 5, or more nucleotides. The mutation can comprise at most 1, 2, 3, 4, or 5 or more nucleotides.

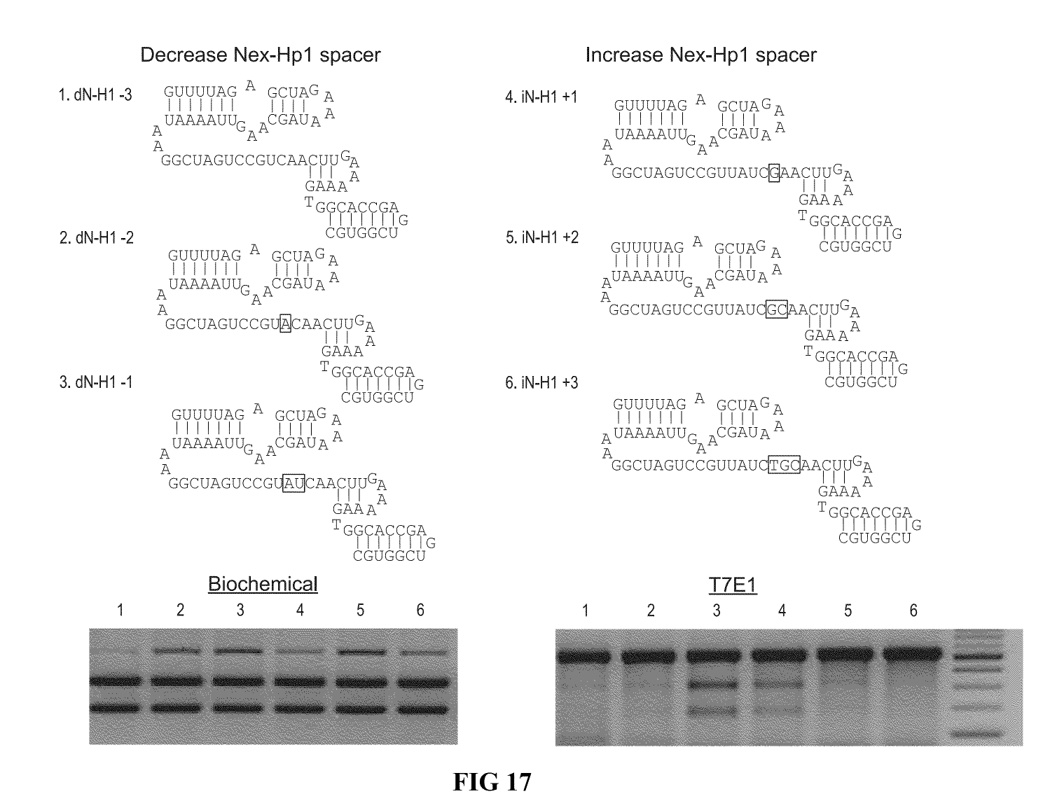

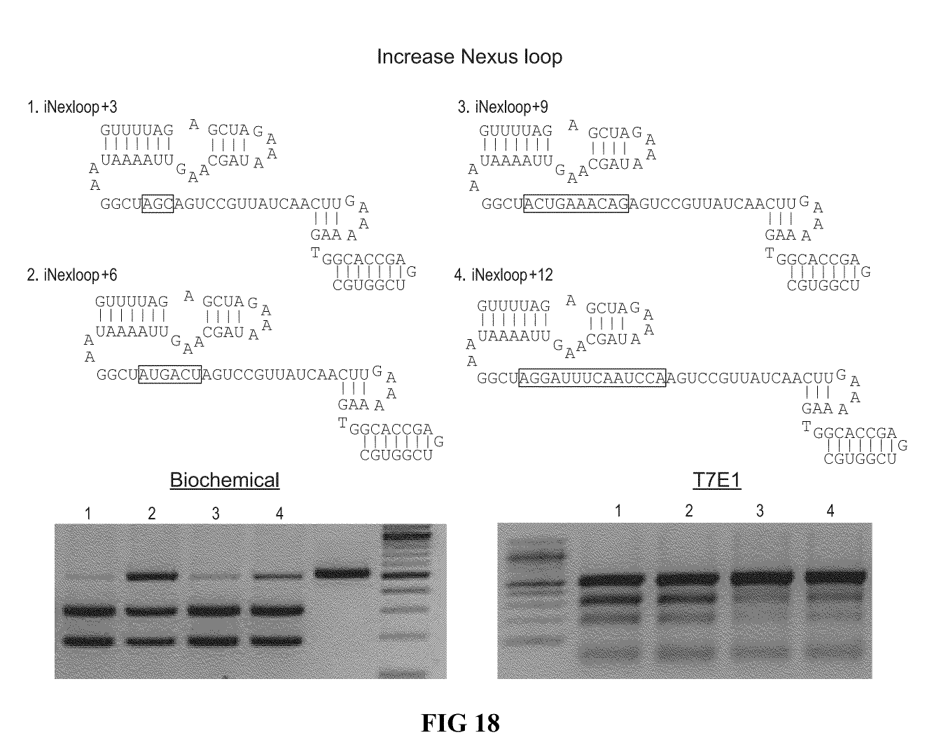

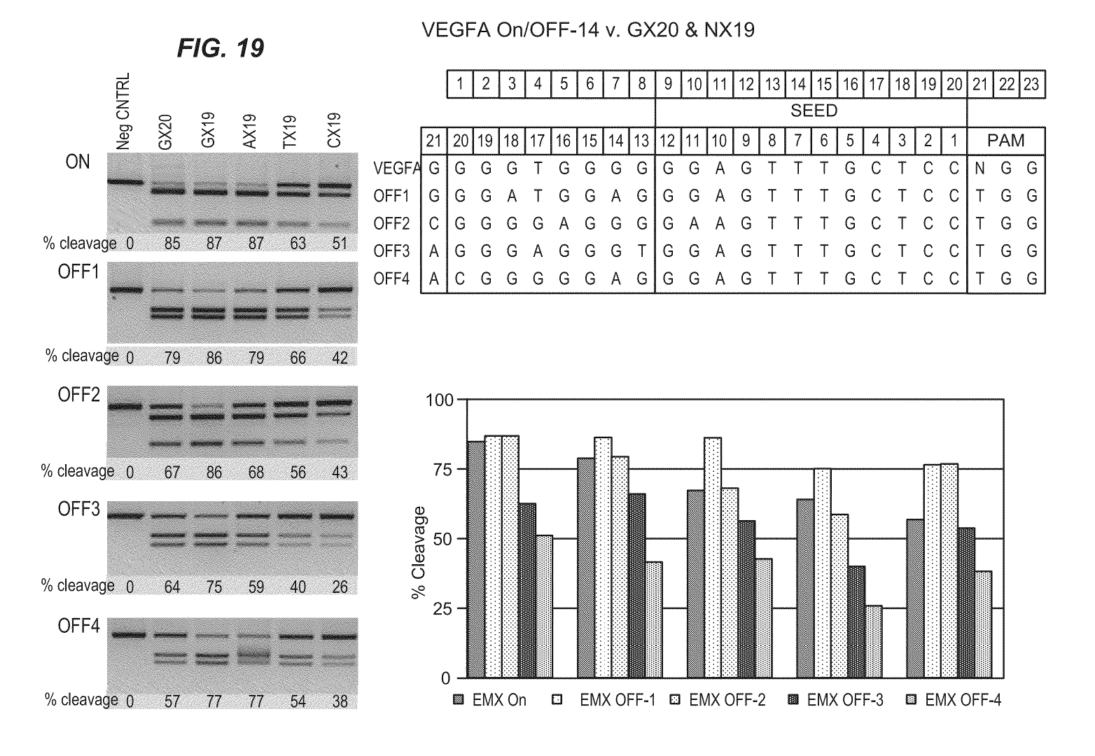

The variants of the nucleic acid-targeting nucleic acids in the library can have variable nexus and/or loop regions of the nucleic acid-targeting nucleic acid. The variants can be tested for activity or effect of the variants in biochemical and cellular assays. FIG. 17 shows the activity of variants to the loop between the nexus and the first hairpin of the hairpin region (e.g., 3′ tracrRNA extension) both in vitro and in vivo. FIG. 18 shows the activity of variants to the nexus loop in vitro and in vivo. Sequences of variants used in these experiments are outlined in Table 5. FIG. 19 shows activity of nucleic acid-targeting nucleic acids engineered in the nexus region, for example, NX19 sgRNA variant.

The libraries can be screened for different parameters such as binding affinity, binding specificity, nucleic acid cleavage activity, homologous recombination activity, and the like.

Mutated nucleic acid-targeting nucleic acids of the library that are selected for an initial property can be further mutated, screened, and selected. Exemplary properties that can be selected for include binding affinity, structural conformation, stability, degradation, cleavage efficiency, and off-target binding. The process of selection can be repeated at least 2, 3, 4, 5, 6, 7, 8, 9, or 10 or more times. The process of selection can be repeated at most 2, 3, 4, 5, 6, 7, 8, 9, or 10 or more times. The process of selection can include, for example, SELEX, directed evolution, and combinatorial biochemistry.

Off-Target Nucleic Acids

The disclosure provides for methods and compositions for reducing off-targeting binding and/or cleavage of target nucleic acids. An off-target nucleic acid can refer to a nucleic acid that is not intended to be bound by a designed or a non-natural nucleic acid-targeting nucleic acid. An off-target region can refer to any region of a nucleic acid, for example, genomic DNA, that is not the target region. An off-target region can refer to any region of a nucleic acid, for example, genomic DNA, other than the target region. An off-target nucleic acid can be a nucleic acid with at least 1, 2, 3, 4, 5, 6, 7, 8, 9, or 10 or more mismatched nucleotides between the off-target nucleic acid and the spacer sequence of a nucleic acid-targeting nucleic acid. An off-target nucleic acid can be a nucleic acid with at most 1, 2, 3, 4, 5, 6, 7, 8, 9, or 10 or more mismatched nucleotides between the off-target nucleic acid and the spacer sequence of a nucleic acid-targeting nucleic acid.

In some embodiments, an off-target nucleic acid can be identical to a target nucleic acid except for, for example, from 1-5 nucleotide substitutions, mutations, and/or deletions compared to the target nucleic acid.

An off-target nucleic acid can be bound by a nucleic acid-targeting nucleic acid of the disclosure with a lower or higher affinity than a target nucleic acid. For example, an off-target nucleic acid can be bound by a nucleic acid-targeting nucleic acid with at least 1-fold, 2-fold, 3-fold, 4-fold, 5-fold, 6-fold, 7-fold, 8-fold, 9-fold, or 10-fold or more lower or higher affinity than a target nucleic acid. An off-target nucleic acid can be bound by a nucleic acid-targeting nucleic acid with at least 1-fold, 2-fold, 3-fold, 4-fold, 5-fold, 6-fold, 7-fold, 8-fold, 9-fold, or 10-fold or more lower or higher affinity than a target nucleic acid.

A nucleic acid-targeting nucleic acid can bind to an on-target or a set of one or more off-target nucleic acids. The set of off-target nucleic acids can be unique for the given nucleic acid-targeting nucleic acid. The off-target nucleic acid for a given nucleic acid-targeting nucleic acid may be the same as an off-target nucleic acid for a different nucleic acid-targeting nucleic acid. The off-target nucleic acid for a given nucleic acid-targeting nucleic acid may be different from an off-target nucleic acid for a different nucleic acid-targeting nucleic acid. The off-target nucleic acid for a given nucleic acid-targeting nucleic acid may overlap with an off-target nucleic acid for a different nucleic acid-targeting nucleic acid.

The percent complementarity between an off-target nucleic acid site and an on-target nucleic acid site can be at least about 1%, at least about 5%, at least about 10%, at least about 20%, at least about 30%, at least about 40%, at least about 50%, at least about 60%, at least about 70%, at least about 80%, at least about 90% or more. The percent complementarity between an off-target nucleic acid and a nucleic acid-targeting nucleic acid can be at most about 1%, at most about 5%, at most about 10%, at most about 20%, at most about 30%, at most about 40%, at most about 50%, at most about 60%, at most about 70%, at least most 80%, at most about 90% or more.

A nucleic acid-targeting nucleic acid can bind with more binding affinity to an on-target nucleic acid than to an off-target nucleic acid. A nucleic acid-targeting nucleic acid can bind with at least 10%, at least 20%, at least 30%, at least 40%, at least 50%, at least 60%, at least 70%, at least 80%, at least 90% or at least 100% more binding affinity to an on-target nucleic acid than to an off-target nucleic acid. A nucleic acid-targeting nucleic acid can bind with at most 10%, at most 20%, at most 30%, at most 40%, at most 50%, at most 60%, at most 70%, at most 80%, at most 90% or at most 100% more binding affinity to an on-target nucleic acid than to an off-target nucleic acid.

A nucleic acid-targeting nucleic acid can bind with less binding affinity to an off-target nucleic acid than to a target nucleic acid. A nucleic acid-targeting nucleic acid can bind with at least 10%, at least 20%, at least 30%, at least 40%, at least 50%, at least 60%, at least 70%, at least 80%, at least 90% or at least 100% less binding affinity to an off-target nucleic acid than to a target nucleic acid. A nucleic acid-targeting nucleic acid can bind with at most 10%, at most 20%, at most 30%, at most 40%, at most 50%, at most 60%, at most 70%, at most 80%, at most 90% or at most 100% less binding affinity to an off-target nucleic acid than to a target nucleic acid.

A complex comprising a site-directed polypeptide and a nucleic acid-targeting nucleic acid can bind with a greater or lesser binding constant to an off-target nucleic acid than to a target nucleic acid. An off-target nucleic acid can bind to a complex comprising a site-directed polypeptide and a nucleic acid-targeting nucleic acid with at least 1-fold, 2-fold, 3-fold, 4-fold, 5-fold, 6-fold, 7-fold, 8-fold, 9-fold, or 10-fold or more greater or lesser binding affinity than a target nucleic acid. An off-target nucleic acid can bind to a complex comprising a site-directed polypeptide and a nucleic acid-targeting nucleic acid with at most 1-fold, 2-fold, 3-fold, 4-fold, 5-fold, 6-fold, 7-fold, 8-fold, 9-fold, or 10-fold or more greater or lesser binding affinity than a target nucleic acid.

Off-Target Activity Measurement

The disclosure provides for methods for determining off-target activity (e.g. number of off-target nucleic acids for a given nucleic acid-targeting nucleic acid). Off-target activity can be determined by computational methods and/or experimental methods.

Computational methods can be used to determine an off-target nucleic acid for a given nucleic acid-targeting nucleic acid. Computational methods can comprise scanning the genomic sequence of a subject. The genomic sequence can be segmented in silico into a plurality of nucleic acid sequences. The segmented nucleic acid sequences can be aligned with the nucleic acid-targeting nucleic acid sequence. A sequence search algorithm can determine one or more off-target nucleic acid sequences by identifying segmented genomic sequences with alignments comprising a defined number of base-pair mismatches with the nucleic acid-targeting nucleic acid. The number of base-pair mismatches between a genomic sequence and a nucleic acid-targeting nucleic acid selected by an algorithm can be user-defined, for example, the algorithm can be programmed to identify off-target sequences with mismatches of up to five base pairs between the genomic sequence and the nucleic acid-targeting nucleic acid. In silico binding algorithms can be used to calculate binding and/or cleavage efficiency of each predicted off-target nucleic acid sequence by a site-directed polypeptide using a weighting scheme. This data can be used to calculate off-target activity for a given nucleic acid-targeting nucleic acid and/or site-directed polypeptide.

Off-target binding activity can be determined by experimental methods. The experimental methods can comprise sequencing a nucleic acid sample contacted by a complex comprising a site-directed polypeptide and a nucleic acid-targeting nucleic acid. The contacted nucleic acid sample can be fixed or crosslinked to stabilize protein-DNA complex. The complex comprising the site-directed polypeptide, the nucleic acid (e.g., target nucleic acid, off-target nucleic acid), and/or the nucleic acid-targeting nucleic acid can be captured from the nucleic acid sample with an affinity tag and/or capture agents. Nucleic acid purification techniques can be used to separate the target nucleic acid from the complex. Nucleic acid purification techniques can include spin column separation, precipitation, and electrophoresis. The nucleic acid can be prepared for sequencing analysis by shearing and ligation of adaptors. Preparation for sequencing analysis can include the generation of sequencing libraries of the eluted target nucleic acid.

Sequence determination methods can include but are not limited to pyrosequencing (for example, as commercialized by 454 Life Sciences, Inc., Branford, Conn.); sequencing by ligation (for example, as commercialized in the SOLiD™ technology, Life Technology, Inc., Carlsbad, Calif.); sequencing by synthesis using modified nucleotides (such as commercialized in TruSeq™ and HiSeg™ technology by Illumina, Inc., San Diego, Calif., HeliScope™ by Helicos Biosciences Corporation, Cambridge, Mass., and PacBio RS by Pacific Biosciences of California, Inc., Menlo Park, Calif.), sequencing by ion detection technologies (Ion Torrent, Inc., South San Francisco, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), capillary sequencing (e.g., such as commercialized in MegaBACE by Molecular Dynamics), electronic sequencing, single molecule sequencing (e.g., such as commercialized in SMRT™ technology by Pacific Biosciences, Menlo Park, Calif.), droplet microfluidic sequencing, sequencing by hybridization (such as commercialized by Affymetrix, Santa Clara, Calif.), bisulfate sequencing, and other known highly parallelized sequencing methods. In some aspects, sequencing is performed by microarray analysis. Sequencing analysis can determine the identity and frequency of an off-target binding site for a given nucleic acid-targeting nucleic acid, by counting the number of times a particular binding site is read. The library of sequenced nucleic acids can include target nucleic acids and off-target nucleic acids.

5′ Engineered Nucleic Acid-Targeting Nucleic Acids

Addition

A 5′ engineered nucleic acid-targeting nucleic acid can comprise 1, 2, 3, 4, 5, or more additional nucleotides on the 5′ end of the nucleic acid-targeting nucleic acid. The additional 5′ nucleotide can be located adjacent to the 5′ end of the spacer of the nucleic acid-targeting nucleic acid.

The 5′ engineered nucleic acid-targeting nucleic acid can comprise 1 additional nucleotide on the 5′ end of the nucleic acid-targeting nucleic acid. The 5′ engineered nucleic acid-targeting nucleic acid can comprise 2 additional nucleotides on the 5′ end of the nucleic acid-targeting nucleic acid. The 5′ engineered nucleic acid-targeting nucleic acid can comprise 3 additional nucleotides on the 5′ end of the nucleic acid-targeting nucleic acid.

The 5′ additional nucleotide can be an adenine. The 5′ additional nucleotide can be a guanine. The 5′ additional nucleotide can be a thymine. The 5′ additional nucleotide can be a cytosine. When there are more than one 5′ additional nucleotides, they can be any type of nucleotide and/or modified nucleotide.

The additional nucleotides can be part of the 5′ spacer extension sequence of the engineered nucleic acid-targeting nucleic acid. In other words, the additional nucleotide can be outside of the spacer, or immediately adjacent to the spacer. The spacer region can be 21 nucleotides in length. The spacer region can be 20 nucleotides in length. The spacer region can be 19 nucleotides in length. The length of both the spacer and the 5′ additional nucleotide can be 22 nucleotides in length. The length of both the spacer and the 5′ additional nucleotide can be 21 nucleotides in length. The length of both the spacer and the 5′ additional nucleotide can be 20 nucleotides in length. For example, an engineered nucleic acid-targeting nucleic acid termed GX19 can refer to a 19 nucleotide spacer plus an additional 5′ nucleotide (G). An engineered nucleic acid-targeting nucleic acid termed GX20 can refer to a 20 nucleotide spacer plus an additional 5′ nucleotide (G), for a total of 21 nucleotides.

The 5′ additional nucleotide of the 5′ engineered nucleic acid-targeting nucleic acid can be complementary to the target nucleic acid. In other words, the one or more 5′ additional nucleotides can be complementary to the one or more nucleotides adjacent to the region to which the spacer hybridizes. The one or more 5′ additional nucleotides may not be complementary to the target nucleic acid.

The one or more 5′ additional nucleotides may decrease the conformational flexibility of the nucleic acid-targeting nucleic acid and site-directed polypeptide complex. A decrease in conformational flexibility may increase specificity and/or decreasing off-targeting binding of the nucleic acid-targeting nucleic acid-site directed polypeptide complex.

A 5′ engineered nucleic acid-targeting nucleic acid comprising one or more 5′ additional nucleotides can bind a target nucleic acid with a greater or lesser binding constant than a wild-type nucleic acid-targeting nucleic acid (e.g., without one or more 5′ additional nucleotides). A 5′ engineered nucleic acid-targeting nucleic acid comprising one or more 5′ additional nucleotides can bind a target nucleic acid with at least 1-fold, 2-fold, 3-fold, 4-fold, 5-fold, 6-fold, 7-fold, 8-fold, 9-fold, or 10-fold or more greater or lesser binding affinity than a nucleic acid-targeting nucleic acid lacking the one or more 5′ additional nucleotides. A 5′ engineered nucleic acid-targeting nucleic acid comprising one or more 5′ additional nucleotides can bind a target nucleic acid with at most 1-fold, 2-fold, 3-fold, 4-fold, 5-fold, 6-fold, 7-fold, 8-fold, 9-fold, or 10-fold or more greater or lesser binding affinity than a nucleic acid-targeting nucleic acid lacking the one or more 5′ additional nucleotides.

A 5′ engineered nucleic acid-targeting nucleic acid comprising one or more 5′ additional nucleotides can reduce off-target binding compared to a nucleic acid-targeting nucleic acid without one or more 5′ additional nucleotides. A 5′ engineered nucleic acid-targeting nucleic acid comprising one or more 5′ additional nucleotides can reduce off-targeting binding by at least 1-fold, 2-fold, 3-fold, 4-fold, 5-fold, 6-fold, 7-fold, 8-fold, 9-fold, or 10-fold or more than a nucleic acid-targeting nucleic acid without one or more 5′ additional nucleotides. A 5′ engineered nucleic acid-targeting nucleic acid comprising one or more 5′ additional nucleotides can reduce off-targeting binding by at most 1-fold, 2-fold, 3-fold, 4-fold, 5-fold, 6-fold, 7-fold, 8-fold, 9-fold, or 10-fold or more than a nucleic acid-targeting nucleic acid without one or more 5′ additional nucleotides.

A 5′ engineered nucleic acid-targeting nucleic acid comprising one or more additional nucleotides can reduce off-target binding and/or cleavage by at least 10, 20, 30, 40, 50, 60, 70, 80, 90 or 100% compared to a nucleic acid-targeting nucleic acid lacking one or more 5′ additional nucleotides. A 5′ engineered nucleic acid-targeting nucleic acid comprising one or more additional nucleotides can reduce off-target binding and/or cleavage by at most 10, 20, 30, 40, 50, 60, 70, 80, 90 or 100% compared to a nucleic acid-targeting nucleic acid lacking one or more 5′ additional nucleotides. A 5′ engineered nucleic acid-targeting nucleic acid comprising one or more additional nucleotides can bind to and/or cleave a target nucleic acid by at least 10, 20, 30, 40, 50, 60, 70, 80, 90 or 100% more compared to an off-target nucleic acid. A 5′ engineered nucleic acid-targeting nucleic acid comprising one or more additional nucleotides can bind to and/or cleave a target nucleic acid by at most 10, 20, 30, 40, 50, 60, 70, 80, 90 or 100% more compared to an off-target nucleic acid.

Deletion

A 5′ engineered nucleic acid-targeting nucleic acid can comprise 1, 2 or 3 deleted nucleotides on the 5′ end of the nucleic acid-targeting nucleic acid. The deleted 5′ nucleotide can be located adjacent to the 5′ end of the spacer of the nucleic acid-targeting nucleic acid.

The 5′ engineered nucleic acid-targeting nucleic acid can comprise 1 deleted nucleotide on the 5′ end of the nucleic acid-targeting nucleic acid. The 5′ engineered nucleic acid-targeting nucleic acid can comprise 2 deleted nucleotides on the 5′ end of the nucleic acid-targeting nucleic acid. The 5′ engineered nucleic acid-targeting nucleic acid can comprise 3 deleted nucleotides on the 5′ end of the nucleic acid-targeting nucleic acid.

The 5′ deleted nucleotide can be an adenine. The 5′ deleted nucleotide can be a guanine. The 5′ deleted nucleotide can be a thymine. The 5′ deleted nucleotide can be a cytosine. When there are more than one 5′ deleted nucleotides, they can be any type of nucleotide and/or modified nucleotide.

The 5′ deleted nucleotide of the 5′ engineered nucleic acid-targeting nucleic acid can be complementary to the target nucleic acid. In other words, the one or more 5′ deleted nucleotides can be complementary to the one or more nucleotides adjacent to the region to which the spacer hybridizes. The one or more 5′ deleted nucleotides may not be complementary to the target nucleic acid.

The one or more 5′ deleted nucleotides may decrease the conformational flexibility of the nucleic acid-targeting nucleic acid and site-directed polypeptide complex. A decrease in conformational flexibility may increase specificity and/or decreasing off-targeting binding of the nucleic acid-targeting nucleic acid-site directed polypeptide complex.

A 5′ engineered nucleic acid-targeting nucleic acid comprising one or more 5′ nucleotide deletions can bind a target nucleic acid with a greater or lesser binding constant than a wild-type nucleic acid-targeting nucleic acid (e.g., without one or more 5′ deleted nucleotides). A 5′ engineered nucleic acid-targeting nucleic acid comprising one or more 5′ nucleotide deletions can bind a target nucleic acid with at least 1-fold, 2-fold, 3-fold, 4-fold, 5-fold, 6-fold, 7-fold, 8-fold, 9-fold, or 10-fold or more greater or lesser binding affinity than a nucleic acid-targeting nucleic acid lacking the one or more 5′ deleted nucleotides. A 5′ engineered nucleic acid-targeting nucleic acid comprising one or more 5′ nucleotide deletions can bind a target nucleic acid with at most 1-fold, 2-fold, 3-fold, 4-fold, 5-fold, 6-fold, 7-fold, 8-fold, 9-fold, or 10-fold or more greater or lesser binding affinity than a nucleic acid-targeting nucleic acid lacking the one or more 5′ deleted nucleotides.

A 5′ engineered nucleic acid-targeting nucleic acid comprising one or more 5′ nucleotide deletions can reduce off-target binding compared to a nucleic acid-targeting nucleic acid without one or more 5′ deleted nucleotides. A 5′ engineered nucleic acid-targeting nucleic acid comprising one or more 5′ nucleotide deletions can reduce off-targeting binding by at least 1-fold, 2-fold, 3-fold, 4-fold, 5-fold, 6-fold, 7-fold, 8-fold, 9-fold, or 10-fold or more than a nucleic acid-targeting nucleic acid without one or more 5′ deleted nucleotides. A 5′ engineered nucleic acid-targeting nucleic acid comprising one or more 5′ nucleotide deletions can reduce off-targeting binding by at most 1-fold, 2-fold, 3-fold, 4-fold, 5-fold, 6-fold, 7-fold, 8-fold, 9-fold, or 10-fold or more than a nucleic acid-targeting nucleic acid without one or more 5′ deleted nucleotides.

A 5′ engineered nucleic acid-targeting nucleic acid comprising a 5′ nucleotide deletion can reduce off-target binding and/or cleavage by at least 10, 20, 30, 40, 50, 60, 70, 80, 90 or 100% compared to a nucleic acid-targeting nucleic acid lacking a 5′ nucleotide deletion. A 5′ engineered nucleic acid-targeting nucleic acid comprising a 5′ nucleotide deletion can reduce off-target binding and/or cleavage by at most 10, 20, 30, 40, 50, 60, 70, 80, 90 or 100% compared to a nucleic acid-targeting nucleic acid lacking a 5′ nucleotide deletion. A 5′ engineered nucleic acid-targeting nucleic acid comprising a 5′ nucleotide deletion can bind to and/or cleave a target nucleic acid by at least 10, 20, 30, 40, 50, 60, 70, 80, 90 or 100% more compared to an off-target nucleic acid. A 5′ engineered nucleic acid-targeting nucleic acid comprising a 5′ nucleotide deletion can bind to and/or cleave a target nucleic acid by at most 10, 20, 30, 40, 50, 60, 70, 80, 90 or 100% more compared to an off-target nucleic acid.

Methods

The disclosure provides for methods for increasing specificity and/or reducing off-targeting binding and modification events by complexes comprising engineered site-directed polypeptide (e.g., Cas9) and an engineered nucleic acid-targeting nucleic acid of the disclosure. A non-natural or engineered nucleic acid-targeting nucleic acid of the disclosure can have a decreased ability to modify, for example, genomic DNA in regions that are not the on-target region.

The methods of the disclosure can include contacting a complex comprising a site-directed polypeptide and a nucleic acid-targeting nucleic acid of the disclosure to a target nucleic acid, wherein the complex contacts the target nucleic acid at least 10, 20, 30, 40, 50, 60, 70, 80, 90 or 100% or more than an off-target nucleic acid. The complex can contact the target nucleic acid at most 10, 20, 30, 40, 50, 60, 70, 80, 90 or 100% or more than an off-target nucleic acid. The complex can bind to a target nucleic acid with a binding affinity at least 1, 2, 3, 4, 5, 6, 7, 8, 9, or 10 or more fold greater or lesser than to an off-target nucleic acid. The complex can bind to a target nucleic acid with a binding affinity at most 1, 2, 3, 4, 5, 6, 7, 8, 9, or 10 or more fold greater or lesser than to an off-target nucleic acid. The complex can contact the target nucleic acid and/or off-target nucleic acid by hybridization between the target nucleic acid and/or off-target nucleic acid and the nucleic acid-targeting nucleic acid of the complex.

The disclosure provides for methods to modify a target nucleic acid using the nucleic acid-targeting nucleic acid of the disclosure. The method can be performed using any of the site-directed polypeptides, nucleic acid-targeting nucleic acids, and complexes of site-directed polypeptides and nucleic acid-targeting nucleic acids as described herein. For example, a target nucleic acid can be contacted with a complex comprising a site-directed polypeptide, an engineered nucleic acid-targeting nucleic acid. The site-directed polypeptide can site-specifically modify the target nucleic acid at and/or around the location targeted by the engineered nucleic acid-targeting nucleic acid. For example, the site-directed polypeptide can cleave the target nucleic acid. The site-directed polypeptide can introduce a double-stranded break into the target nucleic acid. The site-directed polypeptide can introduce a single-stranded break into the target nucleic acid.

The site-directed polypeptide can be a fusion protein that exhibits an enzymatic activity on the target nucleic acid. Exemplary enzymatic activities can include methylation, demethylation, acetylation, deacetylation, ubiquitination, deubiquitination, deamination, alkylation, depurination, oxidation, pyrimidine dimer formation, transposition, recombination, chain elongation, ligation, glycosylation. Phosphorylation, dephosphorylation, adenylation, deadenylation, SUMOylation, deSUMOylation, ribosylation, deribosylation, myristoylation, remodelling, cleavage, oxidoreduction, hydrolation, and isomerization. The site-directed polypeptide can increase transcription of the target nucleic acid. The site-directed polypeptide can decrease transcription of the target nucleic acid.

Non-limiting examples of modifications of a target nucleic acid include double-strand break, single-strand break, insertion of one or more nucleotide, deletion of one or more nucleotide, mutation of one or more nucleotide, insertion of a donor polynucleotide, increase in transcription, decrease in transcription, transgene insertion, and enzymatic modification. Exemplary modifications can include methylation, demethylation, acetylation, deacetylation, ubiquitination, deubiquitination, deamination, alkylation, depurination, oxidation, pyrimidine dimer formation, transposition, recombination, chain elongation, ligation, glycosylation, phosphorylation, dephosphorylation, adenylation, deadenylation, SUMOylation, deSUMOylation, ribosylation, deribosylation, myristoylation, remodelling, cleavage, oxidoreduction, hydrolation, and isomerization. In some embodiments, the modification is cleavage of a target nucleic acid. In some embodiments, the modification is double-strand break. In some embodiments, the modification is deletion of a nucleotide. In some embodiments, the modification is increase or decrease in transcription.

The modification of the target nucleic acid may occur at least 1, 2, 3, 4, 5, 6, 7, 8, 9, 10, 20, 30, 40, 50, 60, 70, 80, 90, 100 or more nucleotides away from the either the 5′ or 3′ end of the target nucleic acid. The modification of the target nucleic acid may occur at most 1, 2, 3, 4, 5, 6, 7, 8, 9, 10, 20, 30, 40, 50, 60, 70, 80, 90, 100 or more nucleotides away from the either the 5′ or 3′ end of the target nucleic acid. The modification can occur on a separate nucleic acid that does not comprise the target nucleic acid (e.g., another chromosome).

In some instances, a donor polynucleotide can be inserted into the target nucleic acid, when the target nucleic acid is cleaved. Donor polynucleotide insertion can be performed by the homologous recombination machinery of the cell. The donor polynucleotide may comprise homology arms that are partially or fully complementary to the regions of the target nucleic acid outside of the break point. The homology arms can be at least 1, 2, 3, 4, 5, 6, 7, 8, 9, 10, 20, 30, 40, 50, 60, 70, 80, 90, or 100 or more nucleotides in length. The homology arms can be at least 1, 2, 3, 4, 5, 6, 7, 8, 9, 10, 20, 30, 40, 50, 60, 70, 80, 90, or 100 or more nucleotides in length. The homology arms can be at least 10, 20, 30, 40, 50, 60, 70, 80, 90 or 100% complementary to the target nucleic acid on either side of the location in which the donor polynucleotide will be inserted.

A non-natural nucleic acid targeting nucleic acid of the disclosure can reduce off-target nucleic acid binding by at least about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid. A non-natural nucleic acid targeting nucleic acid of the disclosure can reduce off-target nucleic acid binding by at most about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid.

A non-natural nucleic acid targeting nucleic acid of the disclosure can reduce off-target nucleic acid cleavage by at least about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid. A non-natural nucleic acid targeting nucleic acid of the disclosure can reduce off-target nucleic acid cleavage by at most about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid.

A non-natural nucleic acid targeting nucleic acid of the disclosure can reduce off-target nucleic acid modification by at least about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid. A non-natural nucleic acid targeting nucleic acid of the disclosure can reduce off-target nucleic acid modification by at most about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid.

A non-natural nucleic acid targeting nucleic acid of the disclosure can increase site-specific binding to a target nucleic acid by at least about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid. A non-natural nucleic acid targeting nucleic acid of the disclosure can increase site-specific binding to a target nucleic acid by at most about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid.

A non-natural nucleic acid targeting nucleic acid of the disclosure can increase site-specific cleavage of a target nucleic acid by at least about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid. A non-natural nucleic acid targeting nucleic acid of the disclosure can increase site-specific cleavage of a target nucleic acid by at most about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid.

A non-natural nucleic acid targeting nucleic acid of the disclosure can increase site-specific modification of a target nucleic acid by at least about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid. A non-natural nucleic acid targeting nucleic acid of the disclosure can increase site-specific modification of a target nucleic acid by at most about 1, about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, or about 100% compared with a control nucleic acid-targeting nucleic acid.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Guide RNA Generation

Guide RNAs were produced by in vitro transcription from double-stranded DNA templates incorporating a T7 promoter at the 5′ end of the spacer sequence.

### Example 2

### DNA Template Generation

Double stranded DNA templates for the production of guide RNAs were assembled by PCR using internal assembly oligonucleotides containing the specific variant sequences and universal outer primer sequences corresponding to the T7 promoter (forward) and the 3′end of the tracrRNA (reverse). Three different assembly reactions were used for the DNA templates. In all cases, the outer primers were used at 640 nM. Inner primer concentrations were used as defined in supplementary table. PCR reactions were set up with Kapa HiFi Hot Start Polymerase and contained 0.5 U of polymerase, 1× reaction buffer, and 0.4 uM dNTPs. PCR assembly reactions were carried out using the following thermal cycling conditions: 95° C. for 2 minutes, 30 cycles of 20 seconds at 98° C., 20 seconds at 62° C., 20 s at 72° C., and a final extension at 72° C. for 2 min. DNA quality was evaluated by agarose gel electrophoresis.

### Example 3

### In Vitro Transcription

Between 0.25-0.5 ug of each DNA template was transcribed using T7 High Yield RNA synthesis Kit (NEB) for ˜16 hours at 37° C. The quality of the transcribed guide RNA was checked by agarose gel electrophoresis (2%, SYBR safe) and guide RNA were diluted 30 fold in water prior to use.

### Example 4

### Target dsDNA Generation

Double-stranded DNA target and/or off-target regions for biochemical assays were amplified by PCR from HEK-293 (ATCC) genomic DNA (gDNA) prepared using QuickExtract DNA Extraction solution (Epicentre). PCR reactions were set up with Kapa HiFi Hot Start polymerase and contained 0.5 U of Polymerase, 1× reaction buffer, 0.4 uM dNTPs, 200 nM forward and reverse primers (see Table S4 for details). 20 ng/uL gDNA in a final volume of 25 uL were used to amplify the target region under the following conditions: 95° C. for 2 minutes, 4 cycles of 20 s at 98° C., 20 s at 70° C., (−2° C./cycle), 20 s at 72° C., followed by 25 cycles of 20 s at 98° C., 20 s at 62° C., 20 s at 72° C., and a final extension at 72° C. for 2 min. PCR products were cleaned up using Spin Smart PCR purification tubes (Denville Scientific) and quantified using NanoDrop 2000 UV-Vis spectrophotometer (Thermo Scientific).

### Example 5

### CAS9 Protein Production

Cas9 protein was produced according to the protocol described in Jinek et al., 2012, concentrated to 2.5 mg/mL and flash frozen in liquid Nitrogen, then stored at −80° C.

### Example 6

### CAS9 Cleavage Assays

Prior to carrying out cleavage assays, guide RNAs were incubated for 2 minutes at 95° C., removed from thermocycler and allowed to equilibrate to room temperature. Cas9 was diluted to a final concentration of 200 nM in reaction buffer (20 mM HEPES, 100 mM KCl, 5 mM MgCl₂, 1 mM DTT, and 5% glycerol at pH 7.4). 1.5 uL of each guide RNA was added to Cas9 and incubated at 37° C. for 10 minutes. Cleavage reactions were initiated by the addition of target and/or off-target DNA to a final concentration of 12.5 nM. Samples were mixed and centrifuged briefly before being incubated for 15 minutes at 37° C. Cleavage reactions were terminated by the addition of Proteinase K (Denville Scientific) at a final concentration of 0.2 ug/uL and 0.44 uL RNase A Solution (SigmaAldrich). Samples were incubated for 20 minutes at 37° C. then 20 minutes at 55° C. 8 uL of the total reaction were evaluated for cleavage activity by agarose gel electrophoresis (2%, SYBR Gold). In the specific case of target DNA used to assess the activity of AAVS1 guide RNAs, the appearance of DNA bands at ˜320 bp and ˜180 bp indicated that cleavage had occurred.

### Example 7

### Cell Culture and Cell Line Generation

HEK-293 cells were purchased from ATCC and cultured in DMEM growth medium (Life Technologies) supplemented with 10% FBS (Fisher Scientific), penicillin and streptomycin (Life Technologies). Cells were maintained at 37° C. in 5% CO₂ in a humidified incubator.

Cas9-expressing cell lines (HEK-293-spCas9) were generated by transfecting HEK-293 cells with a linearized plasmid containing a Cas9-GFP fusion gene expressed under the control of the CMV promoter and a neomycin resistance gene. Cas9-expressing cell lines were generated by transfecting HEK-293 cells (ATCC) in 6-well plates with linearized plasmid using Lipofectamine 2000 (Life Technologies) following the manufacturer's recommended protocol.

Stable Cas9-expressing cells were isolated by culturing cells in the presence of Geneticin (Life Technologies) at 300 ug/ml. Clonal cell lines were generated by culturing drug resistant cells at low density in 10 cm plates and picking individual colonies into a 96-well plate. Clonal cell lines were expanded and assessed for Cas9-GFP expression by visualization of GFP using a fluorescent microscope and by measuring Cas9 cleavage activity of target DNA following transfection with an appropriate engineered nucleic acid-targeting nucleic acids.

### Example 8

### Cell Transfections

Engineered nucleic acid-targeting nucleic acids were transfected into HEK-293-spCas9 cells using the following protocol. Engineered nucleic acid-targeting nucleic acids were diluted 150 fold and 2 uL along with 100 ng copGFP reporter plasmid (Santa Cruz Biotechnology), and 350 ng pBluescript plasmid were mixed with 0.5 uL Lipofectamine 2000 in a total volume of 50 uL serum-free DMEM and incubated for 30 minutes at room temperature in wells of a 96-well plate coated with collagen I. 1×10⁵ Cas9-HEK-293 cells in 100 uL growth medium were added to individual wells containing the transfection complexes. The plate was briefly vortexed and then maintained in a tissue culture incubator for 48 hours.

### Example 9

### Target dsDNA Generation for T7E1 Assay

Genomic DNA (gDNA) was isolated from HEK-293-spCas9 cells 48 hours after engineered nucleic acid-targeting nucleic acid transfection using 100 μL QuickExtract DNA Extraction solution (Epicentre) per well followed by incubation at 37° C. for 10 minutes, 55° C. for 6 minutes and 95° C. for 3 minutes to stop the reaction. gDNA samples were stored at −80° C. DNA for T7E1 assays was generated by PCR amplification of the target AAVS1 locus from isolated gDNA. PCR reactions were set up using 1 uL gDNA as template with Kapa HiFi Hot Start polymerase and containing 0.5 U of Polymerase, 1× reaction buffer, 0.4 uM dNTPs and 300 nM forward and reverse primers in a total volume of 25 uL. Target DNA was amplified using the following conditions: 95° C. for 5 minutes, 4 cycles of 20 s at 98° C., 20 s at 70° C., (−2° C./cycle), 30 s at 72° C., followed by 30 cycles of 15 s at 98° C., 20 s at 62° C., 20 s at 72° C., and a final extension at 72° C. for 1 min.

### Example 10

### T7E1 Assay

PCR amplified target DNA for T7E1 assays was denatured at 95° C. for 10 minutes and then allowed to re-anneal by cooling to 25° C. at −0.5° C./s in a thermal cycler. The re-annealed DNA was incubated with 0.5 uL T7 Endonuclease I in 1×NEBuffer 2 buffer (New England Biolabs) in a total volume of 15 uL for 25 minutes at 37° C. The reaction was stopped by the addition of DNA sample loading buffer and the samples were electrophoresed on a 2% agarose gel. DNA bands were visualized using SYBR® Safe (Life Technologies) and UV illumination.

### Example 11

### Off-Target Activity Measurements

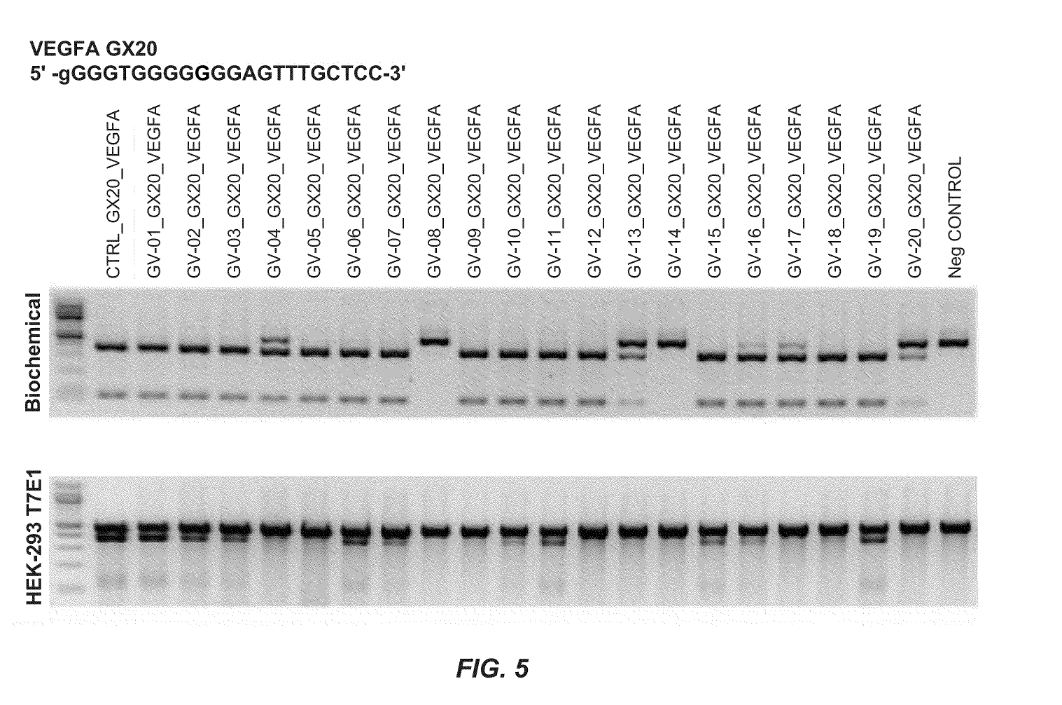

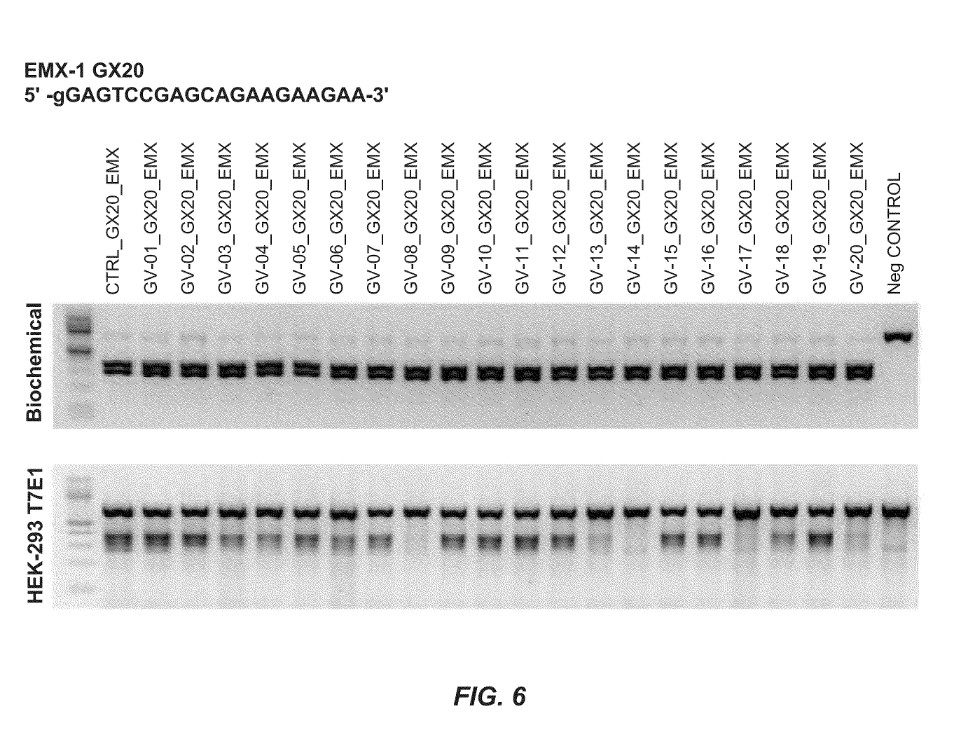

Engineered nucleic acid-targeting nucleic acids to test the effect of structure variants were produced by T7 RNA polymerase-based transcription from a double-stranded DNA template containing a T7 promoter. T7 RNA polymerase prefers a G as the first base of the transcribed RNA (at the 5′ end of the RNA). PCR primers used to construct all the templates for these experiments used a T7 promoter with a G positioned at the 5′ end of the spacer regardless of whether the G is present in the sequence targeted by the spacer or not. In other words, all RNAs transcribed from these templates can generate 21 base spacers with a 5′G (known as GX20 spacers). An alternative nomenclature is that the spacer comprises 20 bases and the 5′ spacer extension comprises a 5′ G. A set of 20 variant nucleic acid-targeting nucleic acids were also synthesized for two additional spacers, one targeting the human VEGFA gene as shown in FIG. 5, and one targeting the human EMX-1 gene as shown in FIG. 6.

The VEGFA engineered nucleic acid-targeting nucleic acids variants showed higher activity in the biochemical assay. Some guide variants were inactive as shown in FIG. 5. Cell-based activity for the VEGFA guide variants followed a similar pattern to the AAVS1 guide variants (FIG. 5). Nearly all EMX-1 guide variants were active biochemically, and most were active in cells (FIG. 6). Data from the EMX-1 nucleic acid-targeting nucleic acid variants indicates that different spacers can affect the ability of Cas9 to bind different nucleic acid-targeting nucleic acid variants and to modulate activity.

Both of the EMX-1 and VEGFA spacers contain a G as the 5′ nucleotide (position 20) within the spacer. By removing the 5′ G from the RNA polymerase template, a version of the spacer can be synthesized that does not have an additional 5′ G, and is 20 nucleotides long (GX19) rather than 21 nucleotides long (GX20).

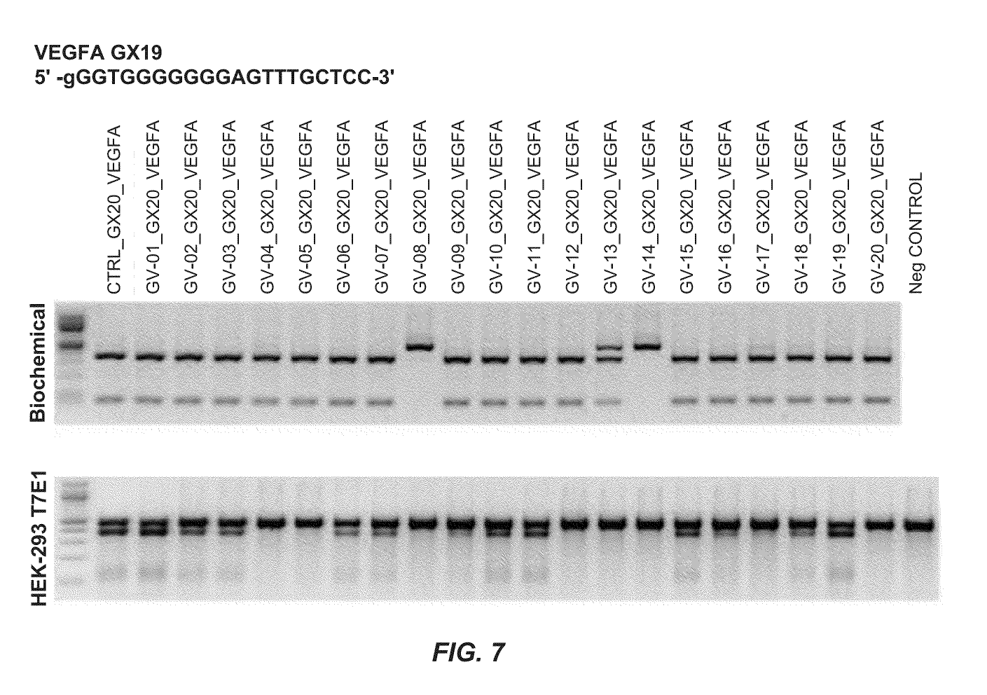

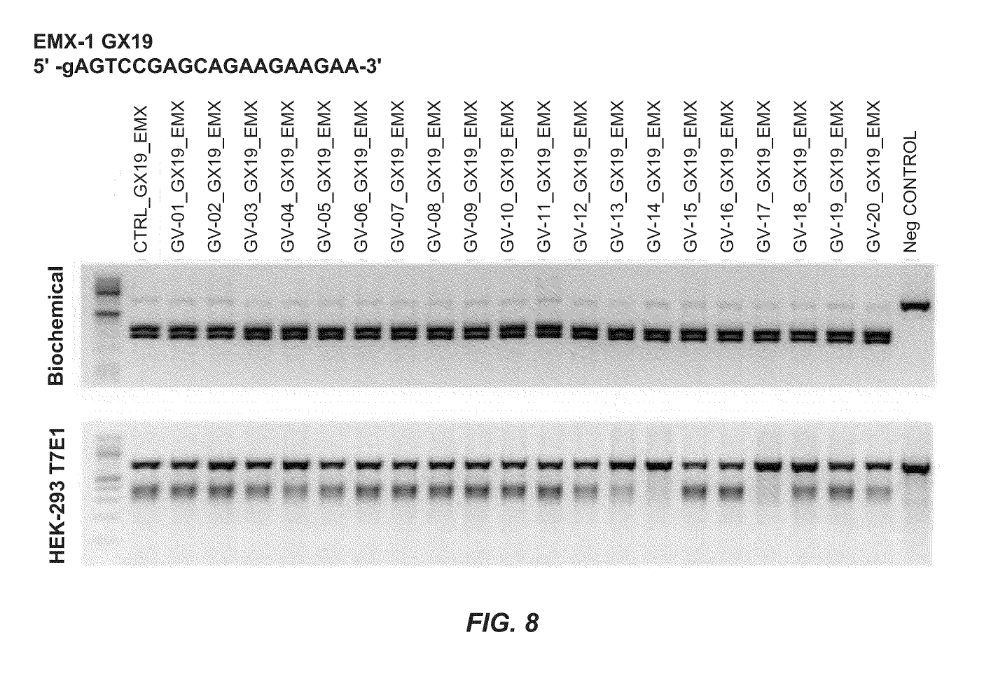

The GX19 nucleic acid-targeting nucleic acids were more active both in biochemical assays and in cells, for both VEGFA and EMX-1 spacers (FIG. 7 and FIG. 8). Some guide variants (e.g. GV-4) that were inactive in all cell-based assays for all spacers demonstrate activity for EMX-1 GX19.

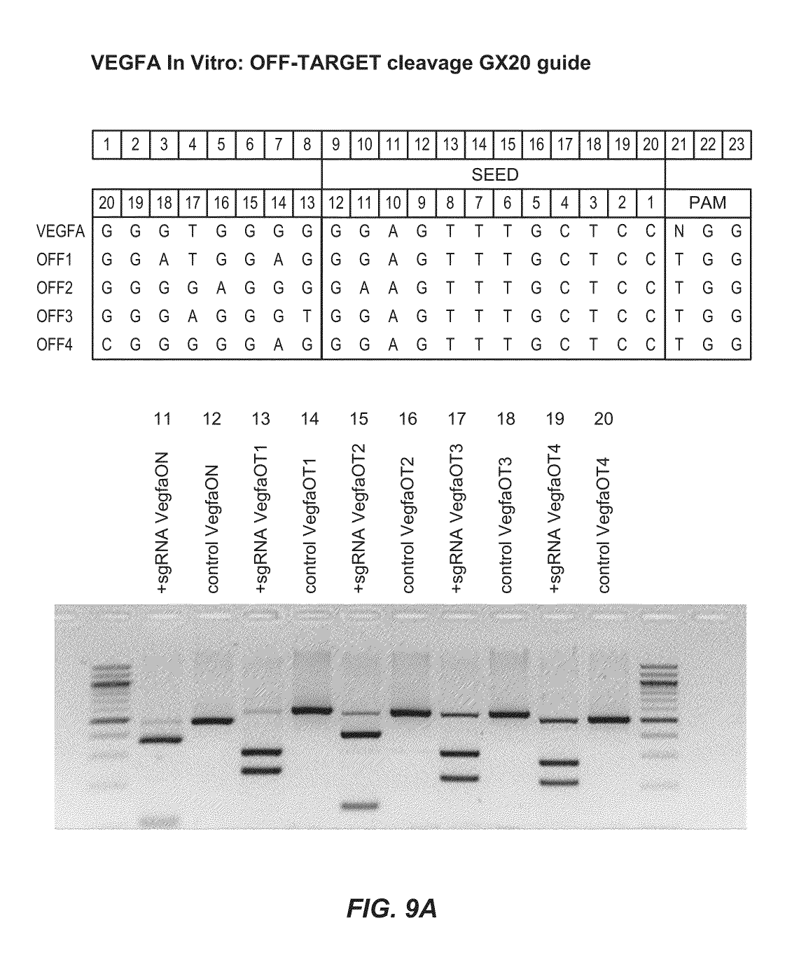

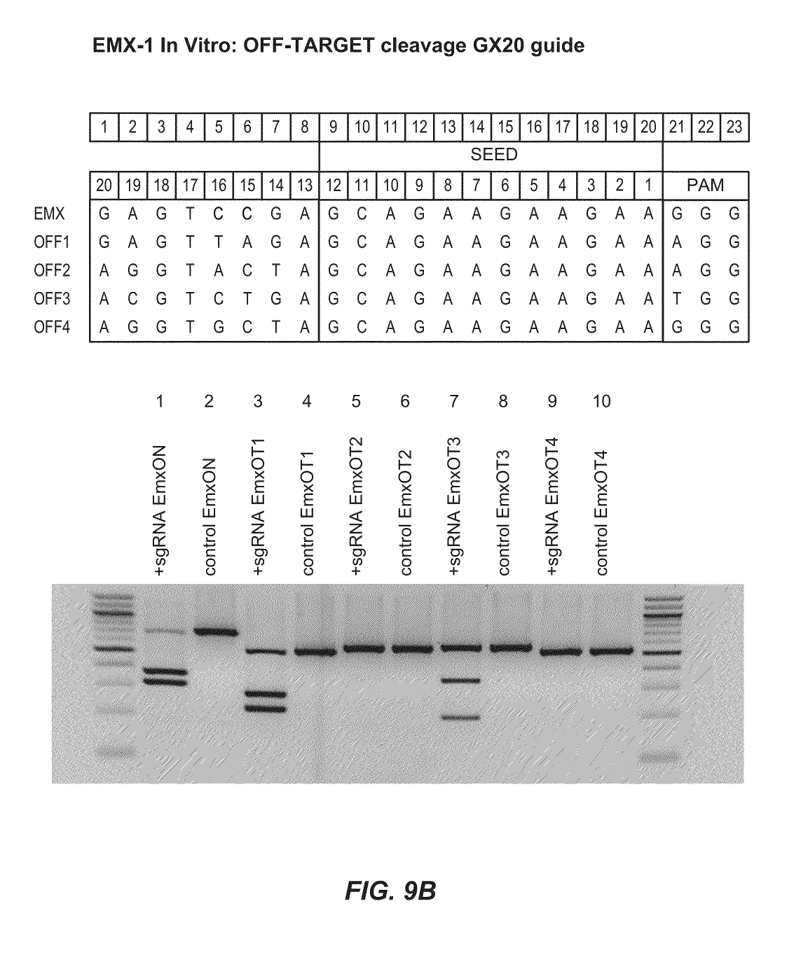

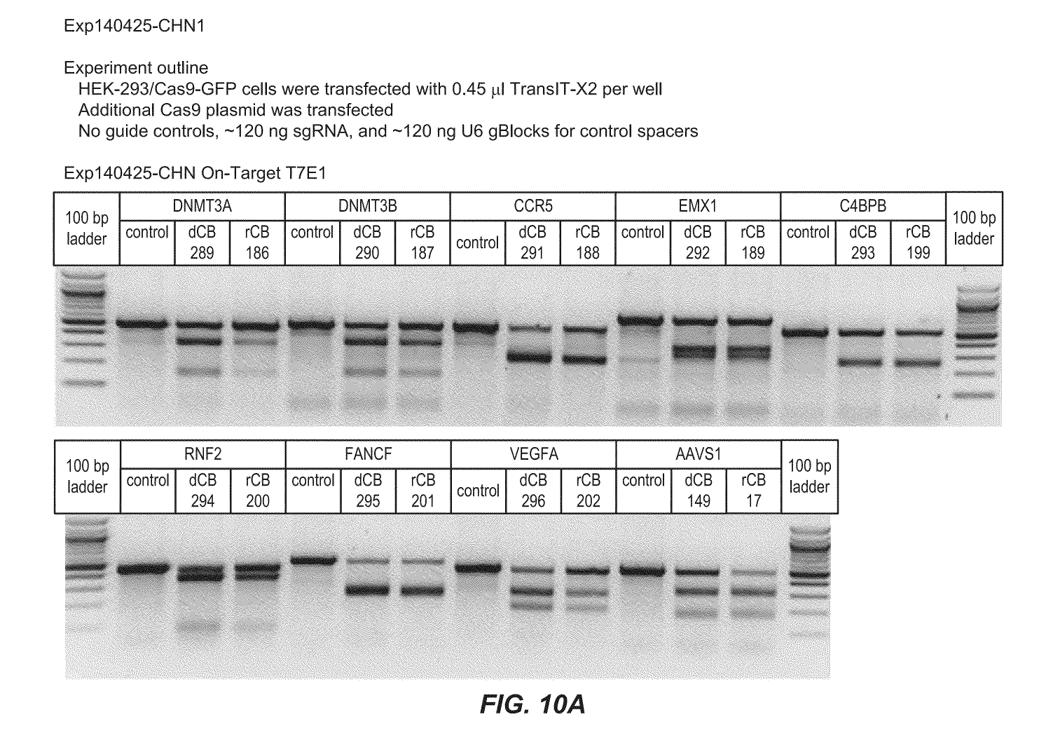

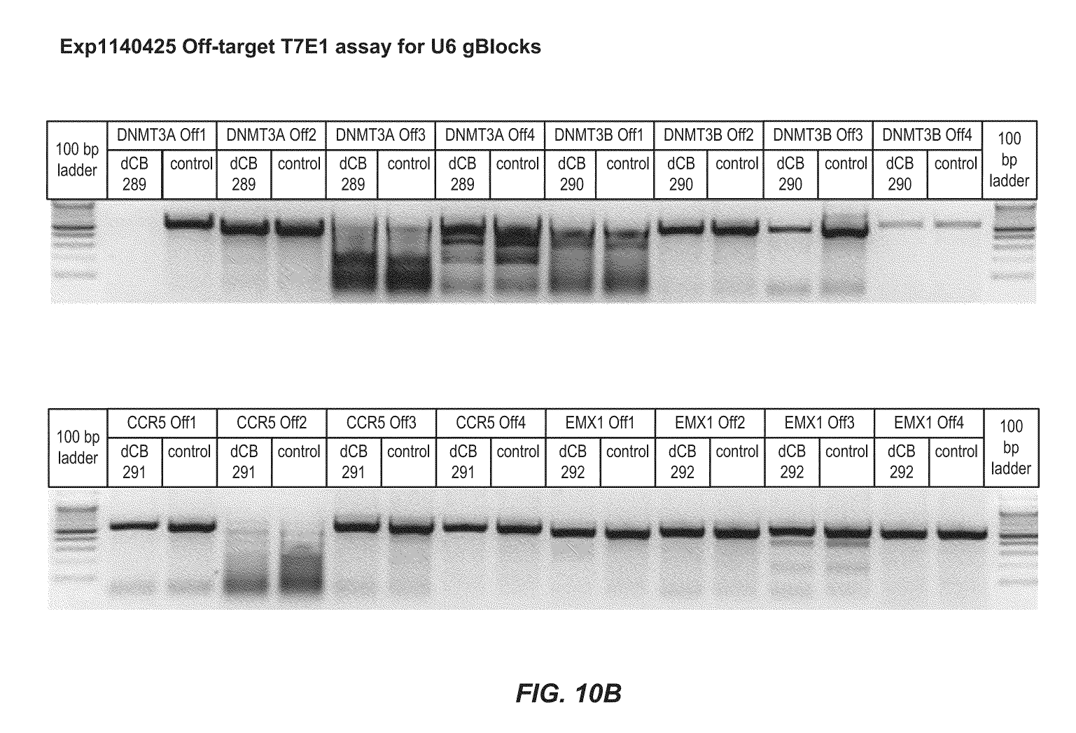

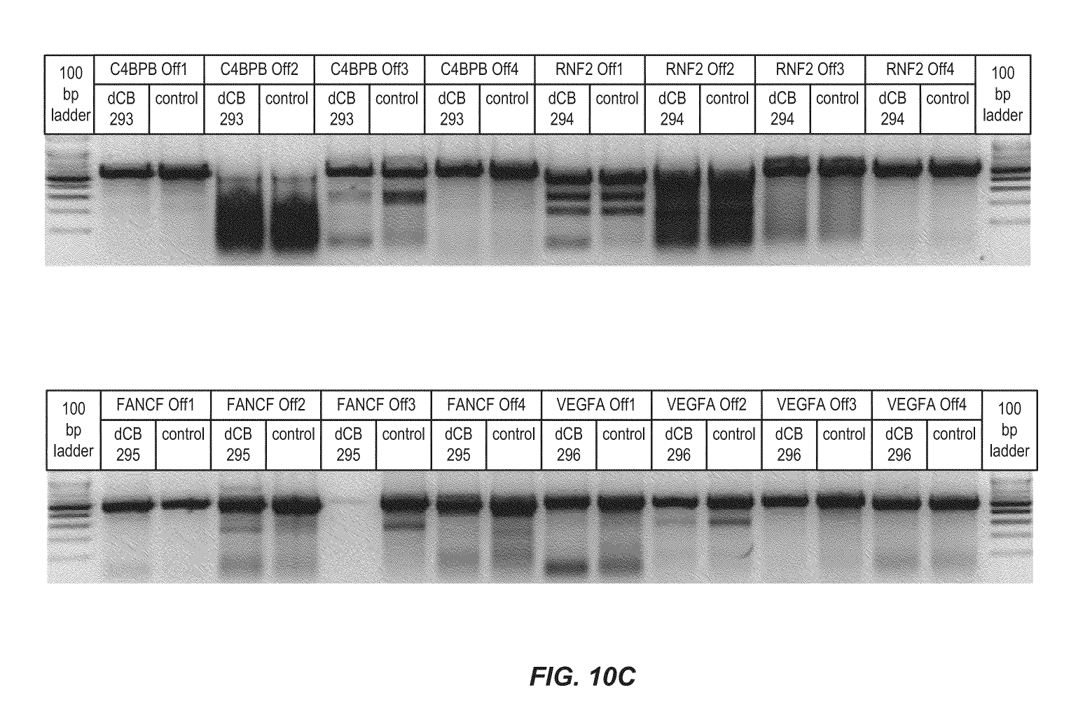

Both VEGFA and EMX-1 target sites are similar in sequence to other sites in the genome. The sequence specificity of a site-directed polypeptide, for example, Cas9, can be imperfect. Off-target test sites were determined by polymerase chain amplification of fragments that contained protospacer sequences similar and/or identical to the spacers of the nucleic acid-targeting nucleic acid of interest. Those sites can be cut biochemically by Cas9 and the GX20 engineered nucleic acid-targeting nucleic acids (FIG. 9A-B). When tested in cells, no off-target activity could be detected for the GX20 engineered nucleic acid-targeting nucleic acids (FIG. 9A-B). This was true for EMX-1, VEGFA, and other sites. Both transcribed RNA and DNA expression cassettes were tested for activity in HEK-293 cells, and while on-target activity remained significant, no activity at off-target sites could be detected by T7E1 assay (FIG. 10A-C). 6 additional sites were also tested and revealed no off target activity (FIGS. 10A-C). In this figure, the target nucleic acid is in the top box (e.g., DNMT3A). dCB refers to DNA expression of the nucleic acid-targeting nucleic acid variant. rCB refers to direct RNA transfection of the nucleic acid-targeting nucleic acid variant. The variant is the number in the box. In some of these experiments, for example FIG. 10B the spacer of the nucleic acid-targeting nucleic acid remains the same but the target nucleic acid is variable (e.g., Off1, Off2, Off3, etc).

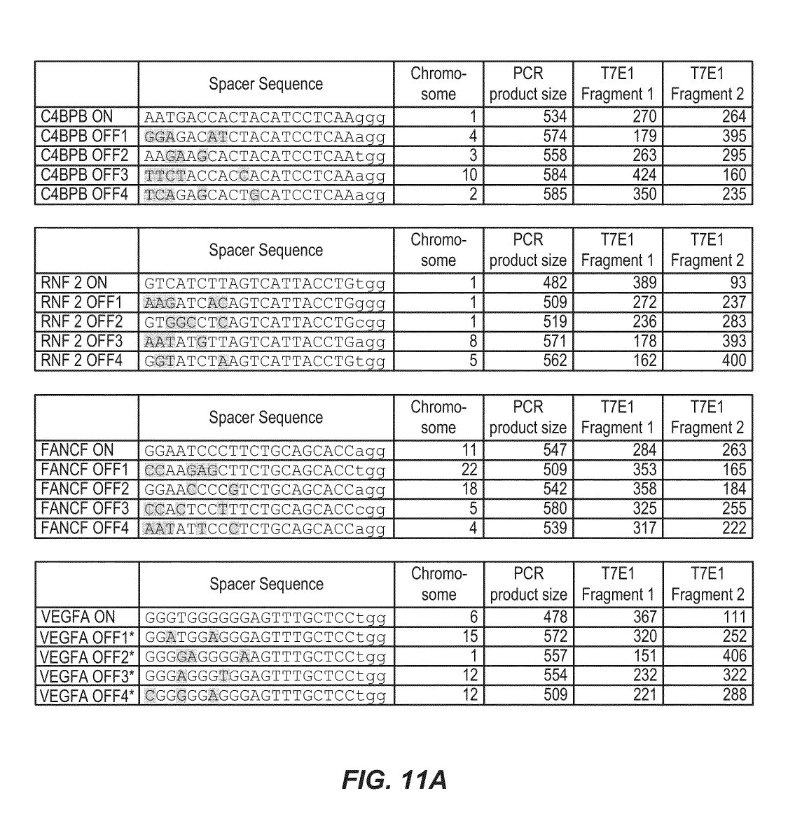

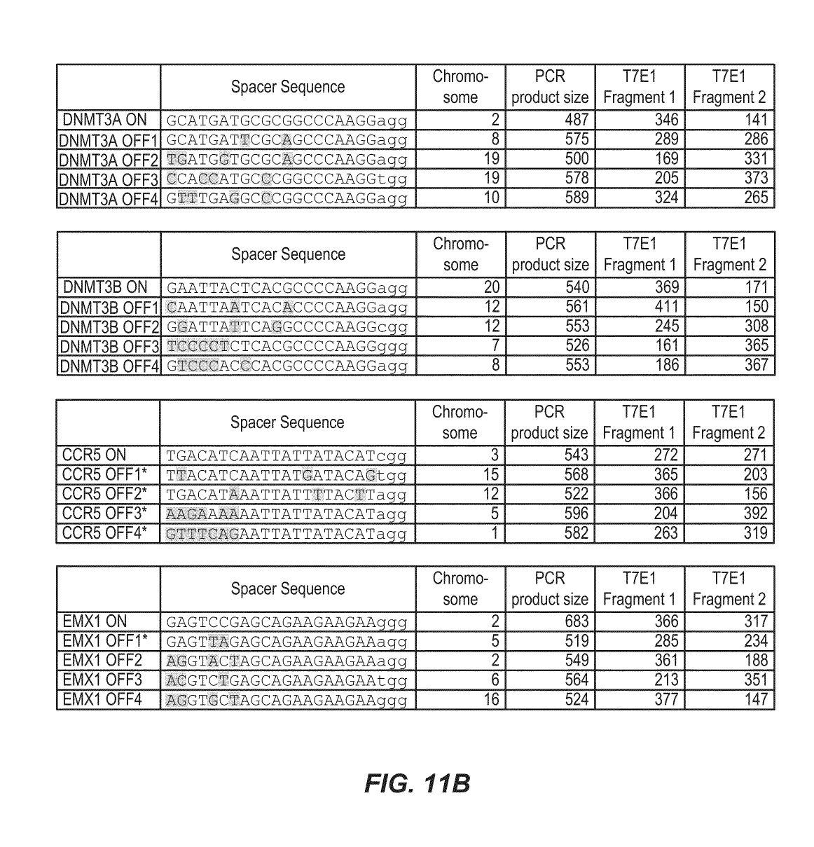

FIGS. 11A and B show on- and off-target sequences for targets in human genome (DNMT3A, DNMT3B, CCR5, EMX-1, C4BPB, RNF2, FANCF, and VEGFA).

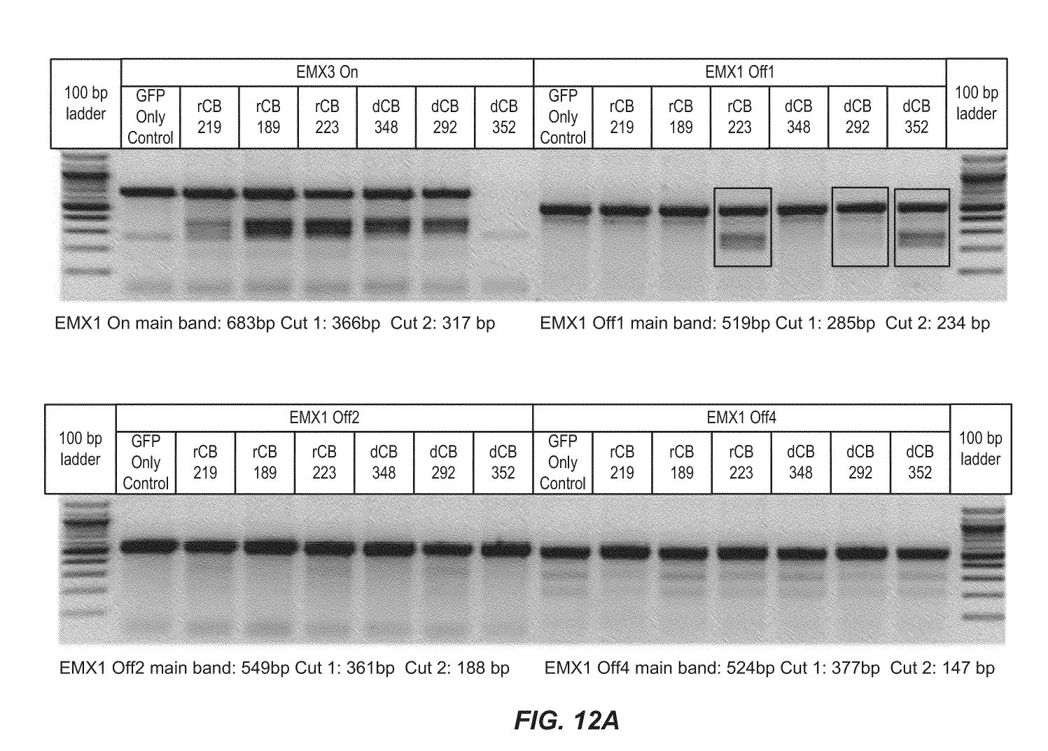

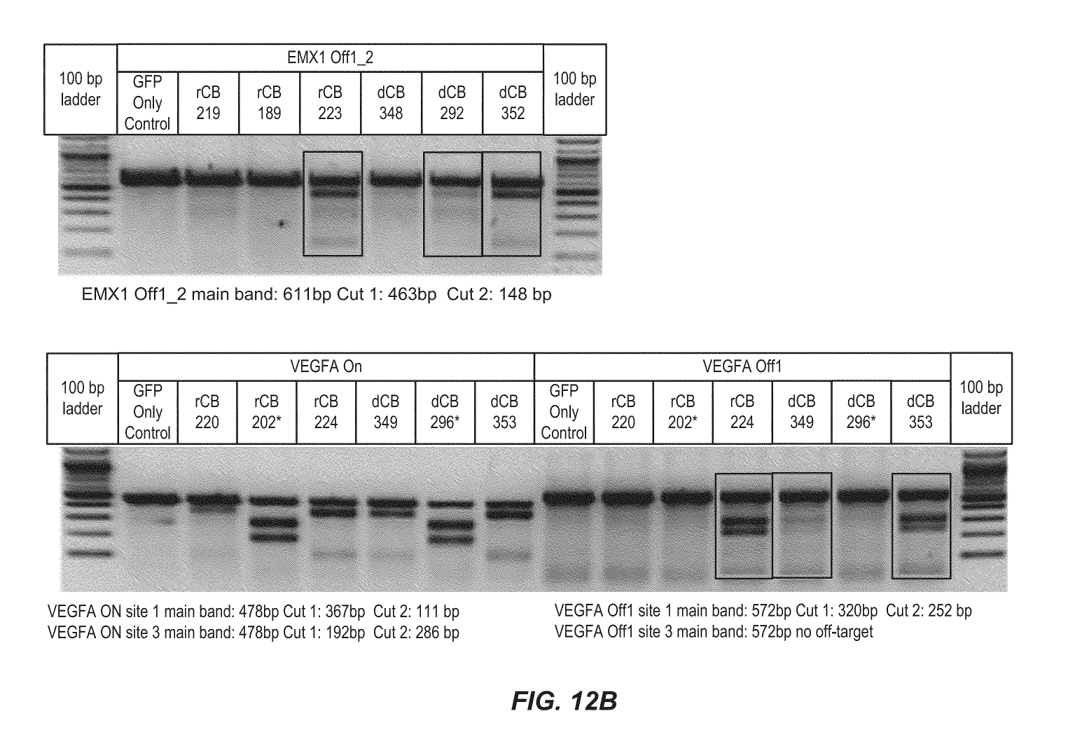

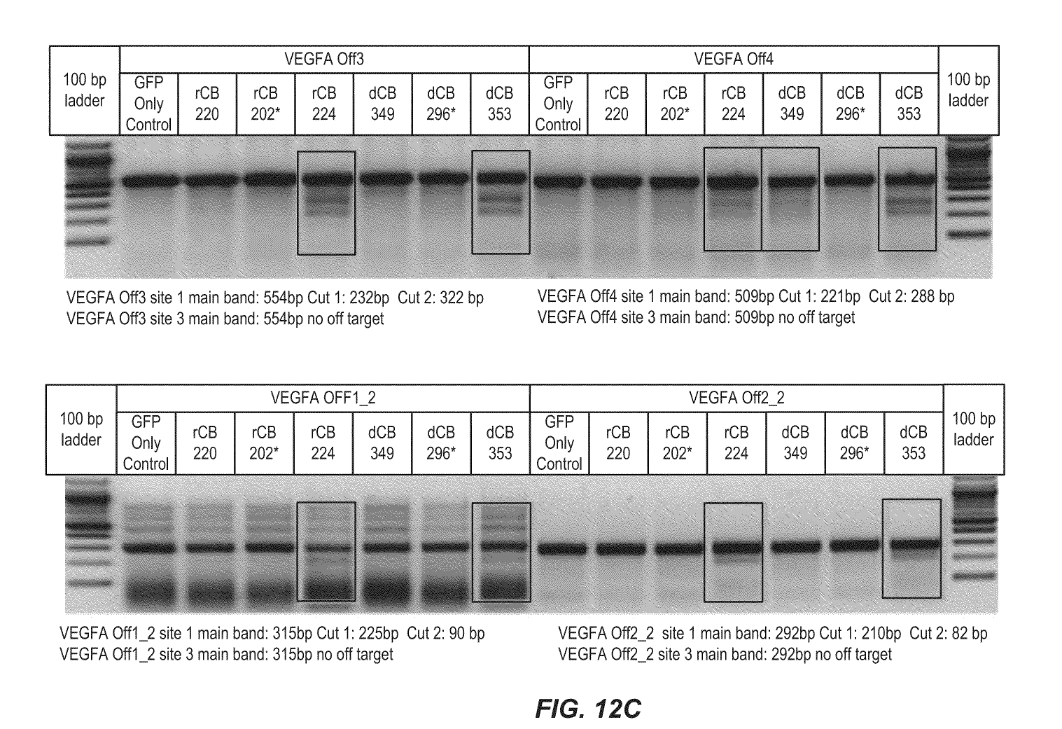

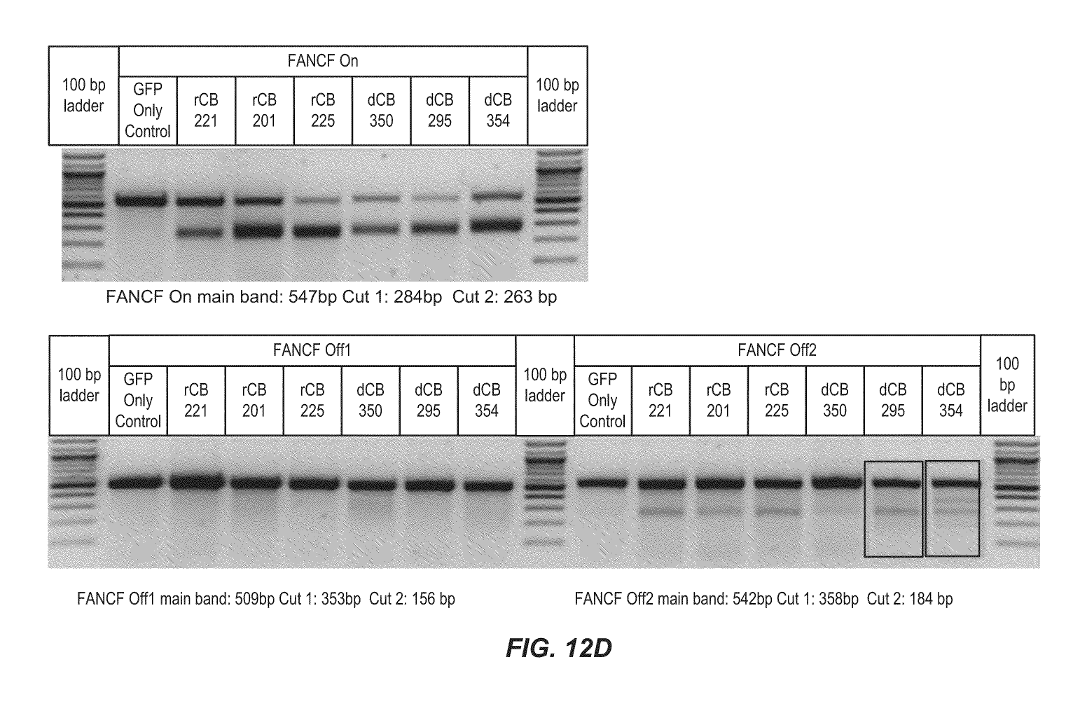

GX19 guides were also tested for off-target activity. Additionally, GGX20 guides were made, in which an additional G was added to the 5′ end to make a 22 base spacer. FIG. 12A-D shows data comparing the activity at on and off-target sites for GX19 guide RNAs targeting spacers from EMX-1, VEGFA, and FANCF. Boxes show conditions with appreciable off-target activity. In these experiments, either RNA injection (r) or DNA expression (d) of engineered nucleic acid-target nucleic acids were evaluated for their ability to cleave an on-target or off-target nucleic acid (EMX1 On, Off1, Off2 etc). For example, for the EMX1 gene, the off-target nucleic acids were determined from sequencing cleavage products from a complex comprising a site-directed polypeptide and EMX1 directed nucleic acid-targeting nucleic acid.

In all cases GX20 guide RNAs show less activity at the off-target site, while maintaining comparable on-target activity. GGX20 guide RNAs also show reduced activity at the off-target site, but on-target activity is also reduced. Addition of a G to the 5′ end of the spacer resulted in reduced off-target activity, with little impact on on-target activity.

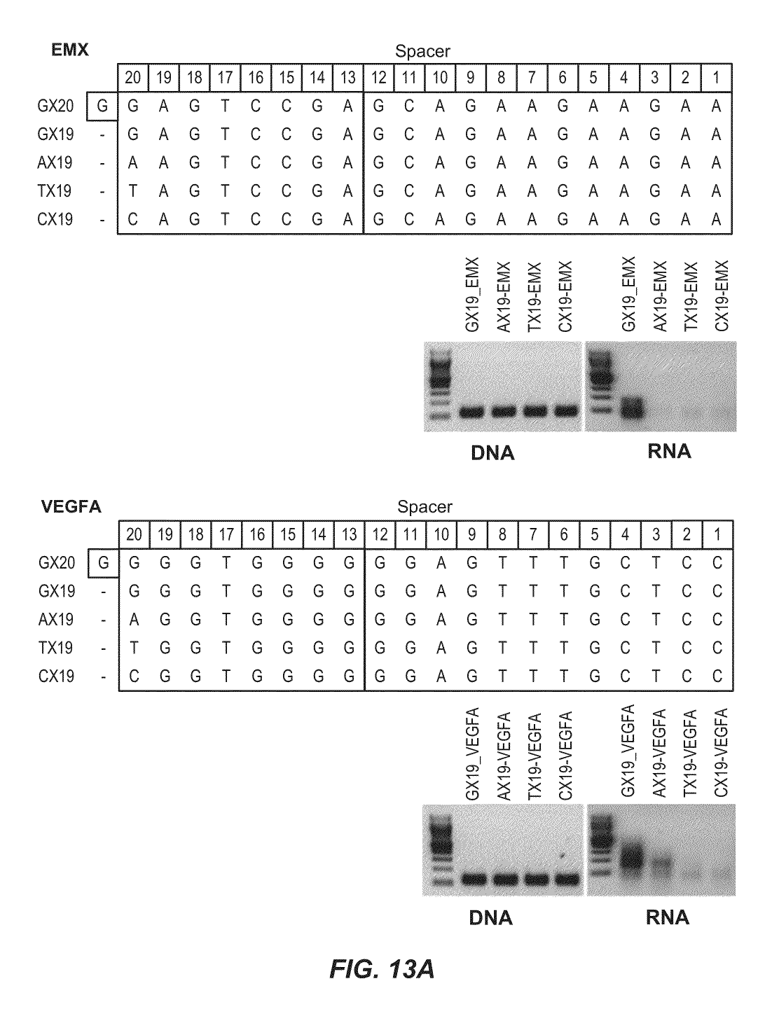

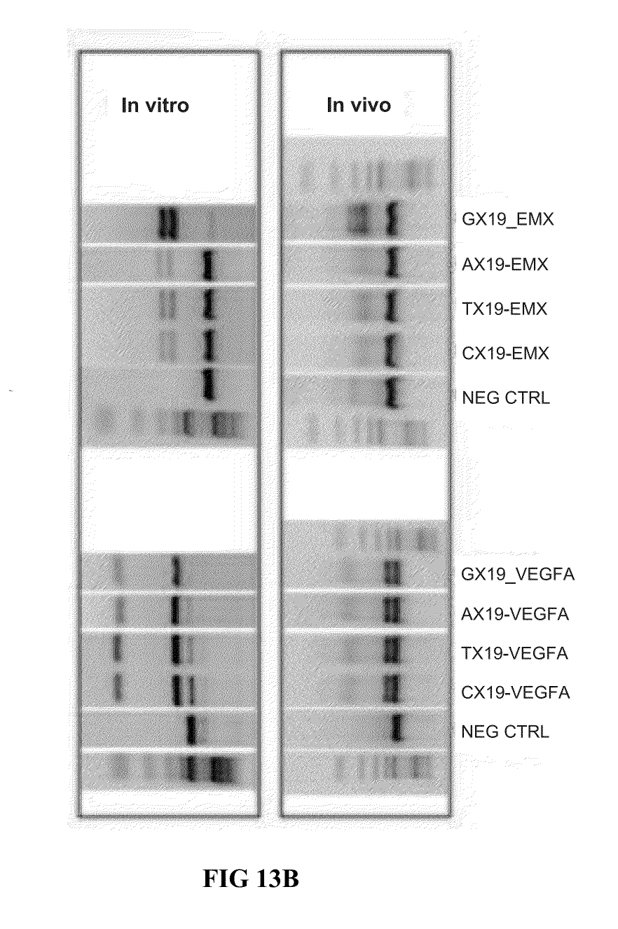

Engineered nucleic acid-targeting nucleic acids for VEGFA and EMX-1 were also made with A, C, or T at the 5′ end and 19 base spacers. Yields for these engineered nucleic acid-targeting nucleic acids were significantly lower than for the GX19 guide RNAs (FIG. 13A). Despite this, for the VEGFA spacer, AX19, CX19, TX19 engineered nucleic acid-targeting nucleic acids have similar on-target activity to the GX19 engineered nucleic acid-targeting nucleic acids (FIG. 13B).

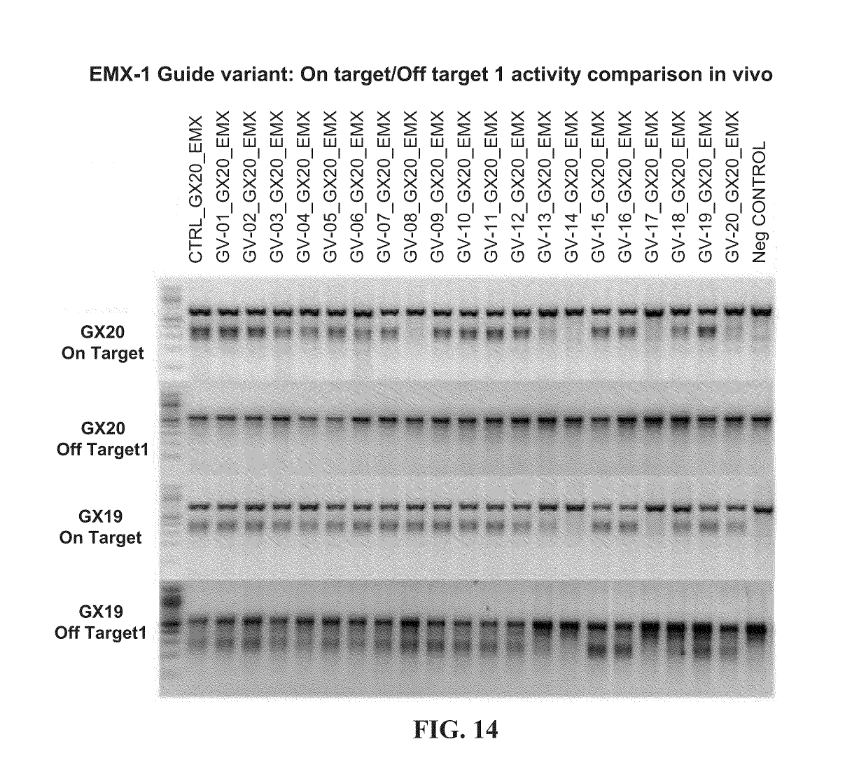

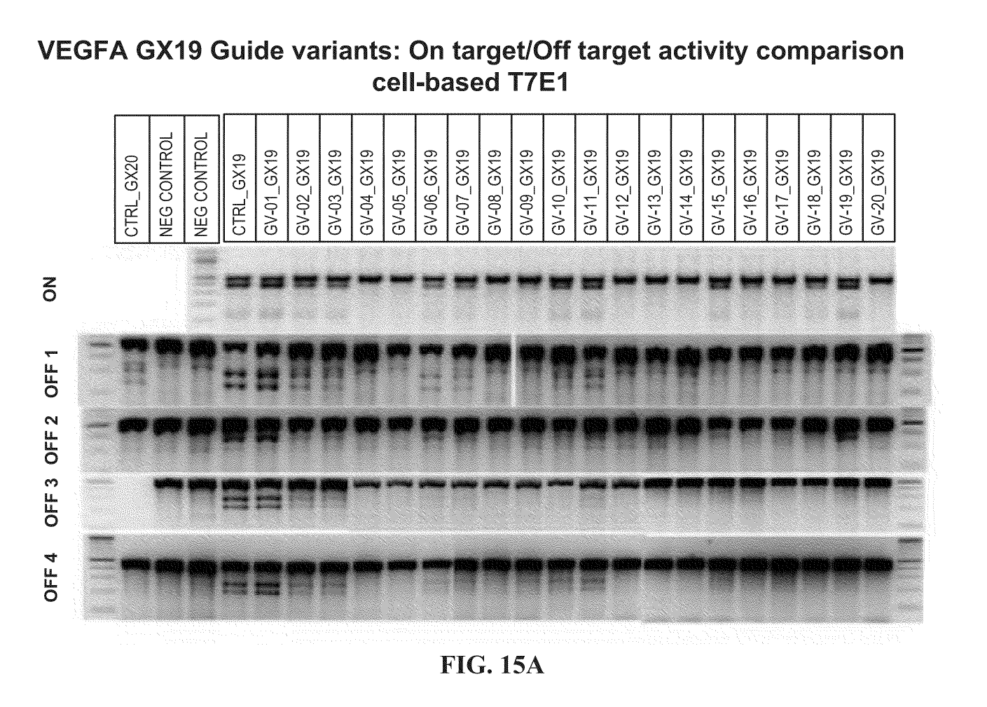

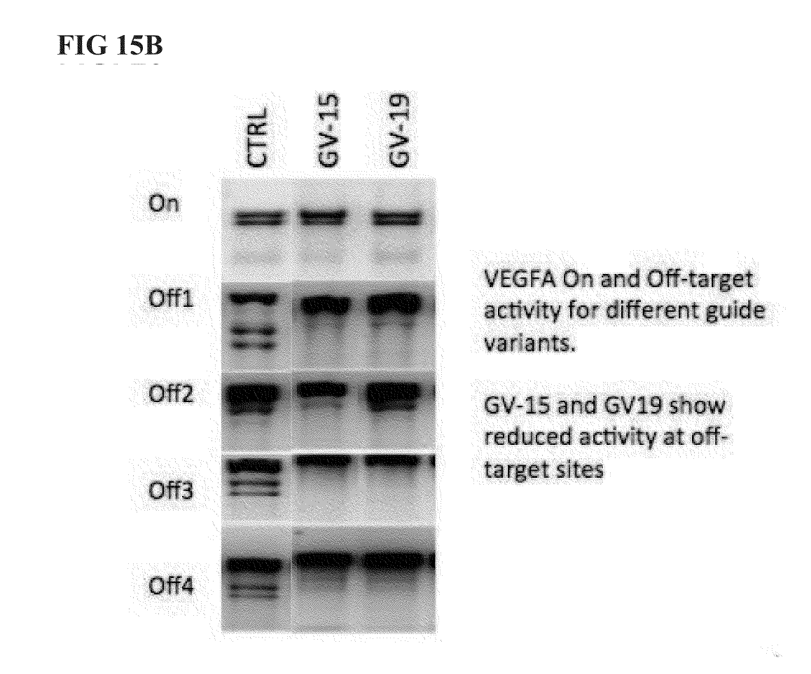

Additional experiments shown in FIG. 14 tested whether different engineered nucleic acid-targeting nucleic acids variants would result in changes to off-target activity. For the EMX-1 spacer, the GX20 spacer showed no activity at off-target sites for any of the 20 engineered nucleic acid-targeting nucleic acids variants tested. In contrast, all engineered nucleic acid-targeting nucleic acids variants for the GX19 spacer showed activity at off-target site 1. For the VEGFA spacer (FIGS. 15A and B), however, certain engineered nucleic acid-targeting nucleic acids variants (e.g. GV-15, GV-19) showed similar on-target activity, but significant reduced off-target activity at all four off-target sites.

### Example 12

### Determination of Activity of Nexus and Loop Nucleic Acid-Targeting Nucleic Acid Variants

Nucleic acid-targeting nucleic acid variants (“sgRNA variants”) comprising nexus and loop mutations were tested in biochemical and cellular assays as shown in FIG. 19.

FIG. 3 shows data generated using a T7E1 assay (in vivo) and using a biochemical assay (in vitro) for a series of variant guide RNA structures (FIG. 3A-B). Variations in the structure of the engineered nucleic acid-targeting nucleic acid backbone while leaving the spacer sequence unchanged can result in changes in nuclease activity at the desired target site both in biochemical assays (FIG. 3C) and in cells (FIG. 3D).

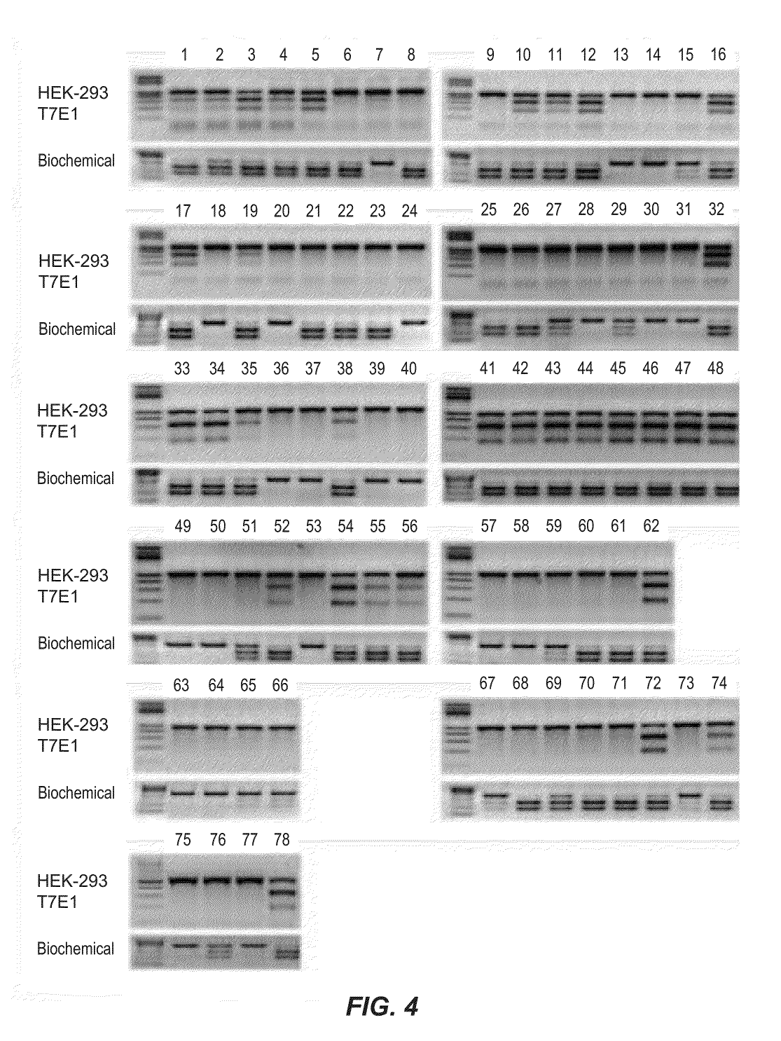

FIG. 4 shows biochemical and cell-based activity data for a further series of variant nucleic acid-targeting nucleic acid structures. The sequences of the variant engineered nucleic acid-targeting nucleic acids in FIG. 3A-D are shown in Table 2. The sequences of the variant engineered nucleic acid-targeting nucleic acids in FIG. 4 are shown in Table 3. Table 4 shows the primer sequences used to construct all the guide variants listed in Table 3, and for which data are shown in FIG. 4.

### Example 13

### Use of a Non-Natural Nucleic Acid-Targeting Nucleic Acid to Reduce Off-Target Activity During Genome Engineering

A first vector(s) encoding a first site-directed polypeptide and an engineered nucleic acid-targeting nucleic acid is introduced into a first group of cells, for example, human cells. A second vector(s) encoding a second site-directed polypeptide and a control nucleic acid-targeting nucleic acid, which lacks an engineered region, is introduced into a second group of cells. Inside the first and second group of cells, the first and second vectors express their elements, respectively. The engineered nucleic acid-targeting nucleic acid forms a first nucleoprotein complex with the first site-directed polypeptide. The control nucleic acid-targeting nucleic acid forms a second nucleoprotein complex with the second site-directed polypeptide. Guided by their respective nucleic-acid targeting nucleic acids, the first and second nucleoprotein complexes bind to and modify the genomic DNA of the first and second group of cells, respectively. The genomic DNA can be modified at target nucleic acids and off-target nucleic acids based on the specificity of the nucleic acid-targeting nucleic acids. In the first group of cells, the genomic DNA is modified at off-target sites, for example, at least 10% less than target sites owing to the engineered non-natural nucleic acid-targeting nucleic acid. The first group of cells also has a lower fraction of cells, for example, at least about 10% less than the second group of cells, with genomic DNA modified at off-target sites or sites other than the target site due to the targeting ability of the engineered nucleic acid-targeting nucleic acid compared with the control nucleic acid-targeting nucleic acid.

In some examples, the engineered nucleic acid-targeting nucleic acid results in, for example, at least about 20%, reduction in off-target binding and modification of the genomic DNA compared with the control nucleic acid-targeting nucleic acid. The fraction of the first group of cells that are modified at off-target sites are, for example, at least about 20% less than the second group of cells.

In some examples, the engineered nucleic acid-targeting nucleic acid results in, for example, about 90%, reduction in off-target binding and modification of the genomic DNA compared with the control nucleic acid-targeting nucleic acid. The fraction of the first group of cells that are modified at off-target sites are, for example, about 90% less than the second group of cells.

In some examples, the engineered nucleic acid-targeting nucleic acid results in, for example, about 95%, reduction in off-target binding and modification of the genomic DNA compared with the control nucleic acid-targeting nucleic acid. The fraction of the first group of cells that are modified at off-target sites are, for example, about 95% less than the second group of cells.

In some examples, the engineered nucleic acid-targeting nucleic acid results in, for example, about 100%, reduction in off-target binding and modification of the genomic DNA compared with the control nucleic acid-targeting nucleic acid. The fraction of the first group of cells that are modified at off-target sites are, for example, about 100% less than the second group of cells.

In some example the first site directed polypeptide is recombinantly expressed and the engineering nucleic acid-targeting nucleic acid is expressed in vitro. The engineered nucleic acid-targeting nucleic acid forms a nucleoprotein complex with the first site-directed polypeptide.

TABLE 4
Guide Variant Template Assembly Primers

| GV | | [Primer | | [Primer | | [Primer | | | | |
|---|---|---|---|---|---|---|---|---|---|---|
| No. | Primer 1 | 1]/nM | Primer 2 | 2]/nM | Primer 3 | 3]/nM | Primer 4 | [Primer 4]/nM | Primer 5 | [Primer 5]/nM |
| GV-1 | AGTAATAATACGA | 640 | TATAGTAATAATACGACTCA | 2 | GGGGCCACTAGGGACAGGATGAA | 0.2 | AAAAAAAGCACCGACTCGGT | 2 | AAAAAAAGCAC | 640 |
| | CTCACTATAG | | CTATAGGGGGCCACTAGGGA | | AAAGAGCTAGAAATAGCAAGTTT | | GCCACTTTTTCAAGTTGATA | | CGACTCGGTGC | |
| | | | CAGGAT | | TTTTAAGGCTAGTCCGTTATCAA | | ACGGACTAGC | | C | |
| | | | | | C | | | | | |
| GV-2 | AGTAATAATACGA | 640 | TATAGTAATAATACGACTCA | 2 | GGGGCCACTAGGGACAGGATGAT | 0.2 | AAAAAAAGCACCGACTCGGT | 2 | AAAAAAAGCAC | 640 |
| | CTCACTATAG | | CTATAGGGGGCCACTAGGGA | | ATAGAGCTAGAAATAGCAAGTTA | | GCCACTTTTTCAAGTTGATA | | CGACTCGGTGC | |
| | | | CAGGAT | | TATTAAGGCTAGTCCGTTATCAA | | ACGGACTAGC | | C | |
| | | | | | C | | | | | |
| GV-3 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGGATGAAAATCCAAGTTA | | GCCACTTTTTCAAGTTGATA | | CGACTCGGTGC | |
| | | | CAGGAT | | AAATAAGGCTAGTCCGTTATCAA | | ACGGACTAGC | | C | |
| | | | | | C | | | | | |
| GV-4 | AGTAATAATACGA | 640 | TATAGTAATAATACGACTCA | 2 | GGGGCCACTAGGGACAGGATGAA | 0.2 | AAAAAAAGCACCGACTCGGT | 2 | AAAAAAAGCAC | 640 |
| | CTCACTATAG | | CTATAGGGGGCCACTAGGGA | | AATGAGGATGAAAATCCAAGTAT | | GCCACTTTTTCAAGTTGATA | | CGACTCGGTGC | |
| | | | CAGGAT | | TTTTAAGGCTAGTCCGTTATCAA | | ACGGACTAGC | | C | |
| | | | | | C | | | | | |
| GV-5 | AGTAATAATACGA | 640 | TATAGTAATAATACGACTCA | 2 | GGGGCCACTAGGGACAGGATGAT | 0.2 | AAAAAAAGCACCGACTCGGT | 2 | AAAAAAAGCAC | 640 |
| | CTCACTATAG | | CTATAGGGGGCCACTAGGGA | | TATGAGGATGAAAATCCAAGTAT | | GCCACTTTTTCAAGTTGATA | | CGACTCGGTGC | |
| | | | CAGGAT | | AATTAAGGCTAGTCCGTTATCAA | | ACGGACTAGC | | C | |
| | | | | | C | | | | | |
| GV-6 | AGTAATAATACGA | 640 | TATAGTAATAATACGACTCA | 2 | GGGGCCACTAGGGACAGGATGTA | 0.2 | AAAAAAAGCACCGACTCGGT | 2 | AAAAAAAGCAC | 640 |
| | CTCACTATAG | | CTATAGGGGGCCACTAGGGA | | ATTGAGGATGAAAATCCAAGTAA | | GCCACTTTTTCAAGTTGATA | | CGACTCGGTGC | |
| | | | CAGGAT | | TTATAAGGCTAGTCCGTTATCAA | | ACGGACTAGC | | C | |
| | | | | | C | | | | | |
| GV-7 | AGTAATAATACGA | 640 | TATAGTAATAATACGACTCA | 2 | GGGGCCACTAGGGACAGGATGAA | 0.2 | AAAAAAAGCACCGACTCGGT | 2 | AAAAAAAGCAC | 640 |
| | CTCACTATAG | | CTATAGGGGGCCACTAGGGA | | AATCAAGTGATGAAAATCGAGAT | | GCCACTTTTTCAAGTTGATA | | CGACTCGGTGC | |
| | | | CAGGAT | | TTTTAAGGCTAGTCCGTTATCAA | | ACGGACTAGC | | C | |
| | | | | | C | | | | | |
| GV-8 | AGTAATAATACGA | 640 | TATAGTAATAATACGACTCA | 2 | GGGGCCACTAGGGACAGGATGAA | 0.2 | AAAAAAAGCACCGACTCGGT | 2 | AAAAAAAGCAC | 640 |
| | CTCACTATAG | | CTATAGGGGGCCACTAGGGA | | AATGAAGGATGAAAATCCAGTAT | | GCCACTTTTTCAAGTTGATA | | CGACTCGGTGC | |
| | | | CAGGAT | | TTTTAAGGCTAGTCCGTTATCAA | | ACGGACTAGC | | C | |
| | | | | | C | | | | | |
| GV-9 | AGTAATAATACGA | 640 | TATAGTAATAATACGACTCA | 2 | GGGGCCACTAGGGACAGGATGAT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AATTAAGGCTAGTCCGTTATCAA | | ACGGACTAGC | | C | |
| | | | | | C | | | | | |
| GV-10 | AGTAATAATACGA | 640 | TATAGTAATAATACGACTCA | 2 | GGGGCCACTAGGGACAGGATGTC | 0.2 | AAAAAAAGCACCGACTCGGT | 2 | AAAAAAAGCAC | 640 |
| | CTCACTATAG | | CTATAGGGGGCCACTAGGGA | | TCAGAGCTAGAAATAGCAAGTTG | | GCCACTTTTTCAAGTTGATA | | CGACTCGGTGC | |
| | | | CAGGAT | | AGATAAGGCTAGTCCGTTATCAA | | ACGGACTAGC | | C | |
| | | | | | C | | | | | |
| GV-11 | AGTAATAATACGA | 640 | TATAGTAATAATACGACTCA | 2 | GGGGCCACTAGGGACAGGATGTC | 0.2 | AAAAAAAGCACCGACTCGGT | 2 | AAAAAAAGCAC | 640 |
| | CTCACTATAG | | CTATAGGGGGCCACTAGGGA | | CCAGAGCTAGAAATAGCAAGTTG | | GCCACTTTTTCAAGTTGATA | | CGACTCGGTGC | |
| | | | CAGGAT | | GGATAAGGCTAGTCCGTTATCAA | | ACGGACTAGC | | C | |
| | | | | | C | | | | | |
| GV-12 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGACTCAGAAATCAGAAGTTA | | GCCACTTTTTCAAGTTGATA | | CGACTCGGTGC | |
| | | | CAGGAT | | AAATAAGGCTAGTCCGTTATCAA | | ACGGACTAGC | | C | |
| | | | | | C | | | | | |
| GV-13 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCTCTAAA | | GCCACTTTTTCAAGTTGATA | | CGACTCGGTGC | |
| | | | CAGGAT | | ATAAGGCTAGTCCGTTATCAAC | | ACGGACTAGC | | C | |
| GV-14 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGGAAACTCTAAAATAAGG | | GCCACTTTTTCAAGTTGATA | | CGACTCGGTGC | |
| | | | CAGGAT | | CTAGTCCGTTATCAAC | | ACGGACTAGC | | C | |
| GV-15 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAAATAAAATAAGGCTAGTC | | GCCACTTTTTCAAGTTGATA | | CGACTCGGTGC | |
| | | | CAGGAT | | CGTTATCAAC | | ACGGACTAGC | | C | |
| GV-16 | AGTAATAATACGA | 640 | TATAGTAATAATACGACTCA | 2 | GGGGCCACTAGGGACAGGATATT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AAATAAGGCTAGTCCGTTATCAA | | ACGGACTAGC | | C | |
| | | | | | C | | | | | |
| GV-17 | AGTAATAATACGA | 640 | TATAGTAATAATACGACTCA | 2 | GGGGCCACTAGGGACAGGATATT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AAACAAGGCTAGTCCGTTATCAA | | ACGGACTAGC | | C | |
| | | | | | C | | | | | |
| GV-18 | AGTAATAATACGA | 640 | TATAGTAATAATACGACTCA | 2 | GGGGCCACTAGGGACAGGATGAC | 0.2 | AAAAAAAGCACCGACTCGGT | 2 | AAAAAAAGCAC | 640 |
| | CTCACTATAG | | CTATAGGGGGCCACTAGGGA | | GATAGAACGGAAACGTTGGACAT | | GCCACTTTTTCAAGTTGATA | | CGACTCGGTGC | |
| | | | CAGGAT | | CGTTAAGGCTAGTCCGTTATCAA | | ACGGACTAGC | | C | |
| | | | | | C | | | | | |
| GV-19 | AGTAATAATACGA | 640 | TATAGTAATAATACGACTCA | 2 | GGGGCCACTAGGGACAGGATGAC | 0.2 | AAAAAAAGCACCGACTCGGT | 2 | AAAAAAAGCAC | 640 |
| | CTCACTATAG | | CTATAGGGGGCCACTAGGGA | | GATGAGACGGAAACGTCAAGTAT | | GCCACTTTTTCAAGTTGATA | | CGACTCGGTGC | |
| | | | CAGGAT | | CGTTAAGGCTAGTCCGTTATCAA | | ACGGACTAGC | | C | |
| | | | | | C | | | | | |
| GV-20 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAAGACTAGAAATAGTGGACTA | | GCCACTTTTTCAAGTTGATA | | CGACTCGGTGC | |
| | | | CAGGAT | | AAATAAGGCTAGTCCGTTATCAA | | ACGGACTAGC | | C | |
| | | | | | C | | | | | |
| GV-21 | AGTAATAATACGA | 640 | TATAGTAATAATACGACTCA | 2 | GGGGCCACTAGGGACAGGATCGT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AAATAAGGCTAGTCCGTTATCAA | | ACGGACTAGC | | C | |
| | | | | | C | | | | | |
| GV-22 | AGTAATAATACGA | 640 | TATAGTAATAATACGACTCA | 2 | GGGGCCACTAGGGACAGGATGTG | 0.2 | AAAAAAAGCACCGACTCGGT | 2 | AAAAAAAGCAC | 640 |
| | CTCACTATAG | | CTATAGGGGGCCACTAGGGA | | GTAGAGCTAGAAATAGCAAGTTA | | GCCACTTTTTCAAGTTGATA | | CGACTCGGTGC | |
| | | | CAGGAT | | AAATAAGGCTAGTCCGTTATCAA | | ACGGACTAGC | | C | |
| | | | | | C | | | | | |
| GV-23 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TGCGAGCTAGAAATAGCAAGTTA | | GCCACTTTTTCAAGTTGATA | | CGACTCGGTGC | |
| | | | CAGGAT | | AAATAAGGCTAGTCCGTTATCAA | | ACGGACTAGC | | C | |
| | | | | | C | | | | | |
| GV-24 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGTGAGAAATAGCAAGTTC | | GCCACTTTTTCAAGTTGATA | | CGACTCGGTGC | |
| | | | CAGGAT | | ACATAAGGCTAGTCCGTTATCAA | | ACGGACTAGC | | C | |
| | | | | | C | | | | | |
| GV-25 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | CACTAAGGCTAGTCCGTTATCAA | | ACGGACTAGC | | C | |
| | | | | | C | | | | | |
| GV-26 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | ACAGAAGGCTAGTCCGTTATCAA | | ACGGACTAGC | | C | |
| | | | | | C | | | | | |
| GV-27 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AAATAACTGGCTAGTCCGTTATC | | ACGGACTAGC | | C | |
| | | | | | AAC | | | | | |
| GV-28 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AAATGCTAGTCCGTTATCAAC | | ACGGACTAGC | | C | |
| GV-29 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AAATAACTCGGCTAGTCCGTTAT | | ACGGACTAGC | | C | |
| | | | | | CAAC | | | | | |
| GV-30 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AAATAACTCTGGCTAGTCCGTTA | | ACGGACTAGC | | C | |
| | | | | | TCAAC | | | | | |
| GV-31 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AAATAACTCTCTGGCTAGTCCGT | | ACGGACTAGC | | C | |
| | | | | | TATCAAC | | | | | |
| GV-32 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AAATAAGGCTAGTCCGTTATCAA | | ACGGACTAGC | | C | |
| | | | | | C | | | | | |
| GV-33 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGGAAACAAGTTAAAATAA | | GCCACTTTTTCAAGTTGATA | | CGACTCGGTGC | |
| | | | CAGGAT | | GGCTAGTCCGTTATCAAC | | ACGGACTAGC | | C | |
| GV-34 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGACAAGTTAAAATAAGGC | | GCCACTTTTTCAAGTTGATA | | CGACTCGGTGC | |
| | | | CAGGAT | | TAGTCCGTTATCAAC | | ACGGACTAGC | | C | |
| GV-35 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGGAGAAACTTTAAAATAAGG | | GCCACTTTTTCAAGTTGATA | | CGACTCGGTGC | |
| | | | CAGGAT | | CTAGTCCGTTATCAAC | | ACGGACTAGC | | C | |
| GV-36 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTATCGAAATCTAAAATAAGGCT | | GCCACTTTTTCAAGTTGATA | | CGACTCGGTGC | |
| | | | CAGGAT | | AGTCCGTTATCAAC | | ACGGACTAGC | | C | |
| GV-37 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTACTTCGGTAAAATAAGGCTAG | | GCCACTTTTTCAAGTTGATA | | CGACTCGGTGC | |
| | | | CAGGAT | | TCCGTTATCAAC | | ACGGACTAGC | | C | |
| GV-38 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGATACTTAAAATAAGGCTAG | | GCCACTTTTTCAAGTTGATA | | CGACTCGGTGC | |
| | | | CAGGAT | | TCCGTTATCAAC | | ACGGACTAGC | | C | |
| GV-39 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTATGAAACTAAAATAAGGCTAG | | GCCACTTTTTCAAGTTGATA | | CGACTCGGTGC | |
| | | | CAGGAT | | TCCGTTATCAAC | | ACGGACTAGC | | C | |
| GV-40 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TCTTCGGAAATAAGGCTAGTCCG | | GCCACTTTTTCAAGTTGATA | | CGACTCGGTGC | |
| | | | CAGGAT | | TTATCAAC | | ACGGACTAGC | | C | |
| GV-41 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGGCTAGAAATAGCAAGTTAA | | GCCACTTTTTCAAGTTGATA | | CGACTCGGTGC | |
| | | | CAGGAT | | AATAAGGCTAGTCCGTTATCAAC | | ACGGACTAGC | | C | |
| GV-42 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGCTAGAAATAGCAAGTTAAA | | GCCACTTTTTCAAGTTGATA | | CGACTCGGTGC | |
| | | | CAGGAT | | ATAAGGCTAGTCCGTTATCAAC | | ACGGACTAGC | | C | |
| GV-43 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTACTAGAAATAGCAAGTTAAAA | | GCCACTTTTTCAAGTTGATA | | CGACTCGGTGC | |
| | | | CAGGAT | | TAAGGCTAGTCCGTTATCAAC | | ACGGACTAGC | | C | |
| GV-44 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAGTTAA | | GCCACTTTTTCAAGTTGATA | | CGACTCGGTGC | |
| | | | CAGGAT | | AATAAGGCTAGTCCGTTATCAAC | | ACGGACTAGC | | C | |
| GV-45 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGAGTTAAA | | GCCACTTTTTCAAGTTGATA | | CGACTCGGTGC | |
| | | | CAGGAT | | ATAAGGCTAGTCCGTTATCAAC | | ACGGACTAGC | | C | |
| GV-46 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAAGTTAAAA | | GCCACTTTTTCAAGTTGATA | | CGACTCGGTGC | |
| | | | CAGGAT | | TAAGGCTAGTCCGTTATCAAC | | ACGGACTAGC | | C | |
| GV-47 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGTTAAAAT | | GCCACTTTTTCAAGTTGATA | | CGACTCGGTGC | |
| | | | CAGGAT | | AAGGCTAGTCCGTTATCAAC | | ACGGACTAGC | | C | |
| GV-48 | AGTAATAATACGA | 640 | TATAGTAATAATACGACTCA | 2 | GGGGCCACTAGGGACAGGATGTT | 0.2 | AAAAAAAGCACCGACTCGGT | 2 | AAAAAAAGCAC | 640 |
| | CTCACTATAG | | CTATAGGGGGCCACTAGGGA | | TTAGAGCTAGAAATAGCAAGTTA | | GCCACTTTTTCAAGTTGATA | | CGACTCGGTGC | |
| | | | CAGGAT | | AAATAAGGCTAGTCCGTTATCAA | | ACGGACTAGC | | C | |
| | | | | | C | | | | | |
| GV-49 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAACGGAC | | | | CGACTCGGTGC | |
| | | | GATGTTTTAGAGCTAGAAAT | | TAGTTCCATTTTAACTTGCTATT | | | | C | |
| | | | AGCAAGTTAA | | TCTAGCTCTA | | | | | |
| GV-50 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAACGGAC | | | | CGACTCGGTGC | |
| | | | GATGTTTTAGAGCTAGAAAT | | TAGCGAAATTTTAACTTGCTATT | | | | C | |
| | | | AGCAAGTTAA | | TCTAGCTCTA | | | | | |
| GV-51 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAACGGAC | | | | CGACTCGGTGC | |
| | | | GATGTTTTAGAGCTAGAAAT | | TTCGCTTATTTTAACTTGCTATT | | | | C | |
| | | | AGCAAGTTAA | | TCTAGCTCTA | | | | | |
| GV-52 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAACGGTG | | | | CGACTCGGTGC | |
| | | | GATGTTTTAGAGCTAGAAAT | | AAGCCTTATTTTAACTTGCTATT | | | | C | |
| | | | AGCAAGTTAA | | TCTAGCTCTA | | | | | |
| GV-53 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAAGCCAC | | | | CGACTCGGTGC | |
| | | | GATGTTTTAGAGCTAGAAAT | | TAGCCTTATTTTAACTTGCTATT | | | | C | |
| | | | AGCAAGTTAA | | TCTAGCTCTA | | | | | |
| GV-54 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGAATTCGGAC | | | | CGACTCGGTGC | |
| | | | GATGTTTTAGAGCTAGAAAT | | TAGCCTTATTTTAACTTGCTATT | | | | C | |
| | | | AGCAAGTTAA | | TCTAGCTCTA | | | | | |
| GV-55 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTACTTAACGGAC | | | | CGACTCGGTGC | |
| | | | GATGTTTTAGAGCTAGAAAT | | TAGCCTTATTTTAACTTGCTATT | | | | C | |
| | | | AGCAAGTTAA | | TCTAGCTCTA | | | | | |
| GV-56 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCATCATGATAACGGAC | | | | CGACTCGGTGC | |
| | | | GATGTTTTAGAGCTAGAAAT | | TAGCCTTATTTTAACTTGCTATT | | | | C | |
| | | | AGCAAGTTAA | | TCTAGCTCTA | | | | | |
| GV-57 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAACGGAC | | | | CGACTCGGTGC | |
| | | | GATGTTTTAGAGCTAGAAAT | | TAGGGTTATTTTAACTTGCTATT | | | | C | |
| | | | AGCAAGTTAA | | TCTAGCTCTA | | | | | |
| GV-58 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAACCCAC | | | | CGACTCGGTGC | |
| | | | GATGTTTTAGAGCTAGAAAT | | TAGGGTTATTTTAACTTGCTATT | | | | C | |
| | | | AGCAAGTTAA | | TCTAGCTCTA | | | | | |
| GV-59 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAACCCAC | | | | CGACTCGGTGC | |
| | | | GATGTTTTAGAGCTAGAAAT | | TAGCCTTATTTTAACTTGCTATT | | | | C | |
| | | | AGCAAGTTAA | | TCTAGCTCTA | | | | | |
| GV-60 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAACTTAC | | | | CGACTCGGTGC | |
| | | | GATGTTTTAGAGCTAGAAAT | | TAGCCTTATTTTAACTTGCTATT | | | | C | |
| | | | AGCAAGTTAA | | TCTAGCTCTA | | | | | |
| GV-61 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAACAAAC | | | | CGACTCGGTGC | |
| | | | GATGTTTTAGAGCTAGAAAT | | TAGCCTTATTTTAACTTGCTATT | | | | C | |
| | | | AGCAAGTTAA | | TCTAGCTCTA | | | | | |
| GV-62 | AGTAATAATACGA | 640 | AGTAATAATACGACTCACTA | 2 | AAAAAAAGCACCGACTCGGTGCC | 2 | N/A | | AAAAAAAGCAC | 640 |
| | CTCACTATAG | | TAGGGGGCCACTAGGGACAG | | ACTTTTTCAAGTTGATAACGAAC | | | | CGACTCGGTGC | |
| | | | GATGTTTTAGAGCTAGAAAT | | TAGTCTTATTTTAACTTGCTATT | | | | C | |
| | | | AGCAAGTTAA | | TCTAGCTCTA | | | | | |
| GV-63 | AGTAATAATACGA | 640 | AGTAATAATACGACTCACTA | 2 | AAAAAACCTTATTTTAACTTGCT | 2 | N/A | | AAAAAAAGCAC | 640 |
| | CTCACTATAG | | TAGGGGGCCACTAGGGACAG | | ATTTCTAGCTCTA | | | | CGACTCGGTGC | |
| | | | GATGTTTTAGAGCTAGAAAT | | | | | | C | |
| | | | AGCAAGTTAA | | | | | | | |
| GV-64 | AGTAATAATACGA | 640 | AGTAATAATACGACTCACTA | 2 | AAAAAACTAGCCTTATTTTAACT | 2 | N/A | | AAAAAAAGCAC | 640 |
| | CTCACTATAG | | TAGGGGGCCACTAGGGACAG | | TGCTATTTCTAGCTCTA | | | | CGACTCGGTGC | |
| | | | GATGTTTTAGAGCTAGAAAT | | | | | | C | |
| | | | AGCAAGTTAA | | | | | | | |
| GV-65 | AGTAATAATACGA | 640 | AGTAATAATACGACTCACTA | 2 | AAAAAACGGACTAGCCTTATTTT | 2 | N/A | | AAAAAAAGCAC | 640 |
| | CTCACTATAG | | TAGGGGGCCACTAGGGACAG | | AACTTGCTATTTCTAGCTCTA | | | | CGACTCGGTGC | |
| | | | GATGTTTTAGAGCTAGAAAT | | | | | | C | |
| | | | AGCAAGTTAA | | | | | | | |
| GV-66 | AGTAATAATACGA | 640 | AGTAATAATACGACTCACTA | 2 | AAAAAAATAACGGACTAGCCTTA | 2 | N/A | | AAAAAAAGCAC | 640 |
| | CTCACTATAG | | TAGGGGGCCACTAGGGACAG | | TTTTAACTTGCTATTTCTAGCTC | | | | CGACTCGGTGC | |
| | | | GATGTTTTAGAGCTAGAAAT | | TA | | | | C | |
| | | | AGCAAGTTAA | | | | | | | |
| GV-67 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACGGGA | | | | | |
| | | | GATGTTTTAGAGCTAGAAAT | | CTAGCCCTTATTTTAACTTGCTA | | | | | |
| | | | AGCAAGTTAA | | TTTCTAGCTCTA | | | | | |
| GV-68 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACGGGG | | | | | |
| | | | GATGTTTTAGAGCTAGAAAT | | ACTAGCCCCTTATTTTAACTTGC | | | | | |
| | | | AGCAAGTTAA | | TATTTCTAGCTCTA | | | | | |
| GV-69 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACGGGG | | | | | |
| | | | GATGTTTTAGAGCTAGAAAT | | GACTAGCCCCCTTATTTTAACTT | | | | | |
| | | | AGCAAGTTAA | | GCTATTTCTAGCTCTA | | | | | |
| GV-70 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACAAAC | | | | | |
| | | | GATGTTTTAGAGCTAGAAAT | | TAGTTTTATTTTAACTTGCTATT | | | | | |
| | | | AGCAAGTTAA | | TCTAGCTCTA | | | | | |
| GV-71 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACGGAA | | | | | |
| | | | GATGTTTTAGAGCTAGAAAT | | CTAGTCCTTATTTTAACTTGCTA | | | | | |
| | | | AGCAAGTTAA | | TTTCTAGCTCTA | | | | | |
| GV-72 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACGGAC | | | | | |
| | | | GATGTTTTAGAGCTAGAAAT | | TAGCCTTATTTTAACTTGCTATT | | | | | |
| | | | AGCAAGTTAA | | TCTAGCTCTA | | | | | |
| GV-73 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACAGGA | | | | | |
| | | | GATGTTTTAGAGCTAGAAAT | | CTAGCCTTATTTTAACTTGCTAT | | | | | |
| | | | AGCAAGTTAA | | TTCTAGCTCTA | | | | | |
| GV-74 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACAGAC | | | | | |
| | | | GATGTTTTAGAGCTAGAAAT | | TAGCTTTATTTTAACTTGCTATT | | | | | |
| | | | AGCAAGTTAA | | TCTAGCTCTA | | | | | |
| GV-75 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACGGGA | | | | | |
| | | | GATGTTTTAGAGCTAGAAAT | | CTAGACCTTATTTTAACTTGCTA | | | | | |
| | | | AGCAAGTTAA | | TTTCTAGCTCTA | | | | | |
| GV-76 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACGGCG | | | | | |
| | | | GATGTTTTAGAGCTAGAAAT | | ACTAGTACCTTATTTTAACTTGC | | | | | |
| | | | AGCAAGTTAA | | TATTTCTAGCTCTA | | | | | |
| GV-77 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACGGCG | | | | | |
| | | | GATGTTTTAGAGCTAGAAAT | | CACTAGATACCTTATTTTAACTT | | | | | |
| | | | AGCAAGTTAA | | GCTATTTCTAGCTCTA | | | | | |
| GV-78 | AGTAATAATACGA | 640 | AGTAATAATACGACTCACTA | 2 | AAAAAAAGCACCGACTCGGTGCC | 640 | N/A | | N/A | |
| | CTCACTATAG | | TAGGGGGCCACTAGGGACAG | | ACTTTTTCAAGTTGATAACGGAC | | | | | |
| | | | GATGTTTTAGAGCTAGAAAT | | TAGCCTTATTTTAACTTGCTATT | | | | | |
| | | | AGCAAGTTAA | | TCTAGCTCTA | | | | | |
| GV-79 | AGTAATAATACGA | 640 | AGTAATAATACGACTCACTA | 2 | GTTTTAGAGCTAGAAATAGCAAG | 2 | N/A | AAAAAAAGC | 640 | |
| | CTCACTATAG | | TAGGGGGCCACTAGGGACAG | | TTAAAATAAGGCTAGTCCGTTAT | | | ACCGACTCG | | |
| | | | GATGTTTTAGAGCTAGAAAT | | CAATGGCACCGAGTCGGTGCT | | | GTGCC | | |
| | | | AGCAAGTTAA | | | | | | | |
| GV-80 | AGTAATAATACGA | 640 | AGTAATAATACGACTCACTA | 2 | GTTTTAGAGCTAGAAATAGCAAG | 2 | N/A | AAAAAAAGC | 640 | |
| | CTCACTATAG | | TAGGGGGCCACTAGGGACAG | | TTAAAATAAGGCTAGTCCGTTAT | | | ACCGACTCG | | |
| | | | GATGTTTTAGAGCTAGAAAT | | CAACTGGCACCGAGTCGGTGCT | | | GTGCC | | |
| | | | AGCAAGTTAA | | | | | | | |
| GV-81 | AGTAATAATACGA | 640 | AGTAATAATACGACTCACTA | 2 | GTTTTAGAGCTAGAAATAGCAAG | 2 | N/A | AAAAAAAGC | 640 | |
| | CTCACTATAG | | TAGGGGGCCACTAGGGACAG | | TTAAAATAAGGCTAGTCCGTTAT | | | ACCGACTCG | | |
| | | | GATGTTTTAGAGCTAGAAAT | | CAACTTGGCACCGAGTCGGTGCT | | | GTGCC | | |
| | | | AGCAAGTTAA | | | | | | | |

TABLE 5

| NAME | SEQ |
|---|---|
| Delete | AGTAATAATACGACTCACTATAGGGGGCCACTAGGGACAGGATGTTTTAGAGCTAGAA |
| Hairpin1 | ATAGCAAGTTAAAATAAGGCTAGTCCGTTATCAATGGCACCGAGTCGGTGCTTTTTTT |
| −0 | AGTAATAATACGACTCACTATAGGGGGCCACTAGGGACAGGATGTTTTAGAGCTAGAA |
| Delete | ATAGCAAGTTAAAATAAGGCTAGTCCGTTATCAACTGGCACCGAGTCGGTGCTTTTTT |
| Hairpin1 | T |
| +1 | |
| Delete | AGTAATAATACGACTCACTATAGGGGGCCACTAGGGACAGGATGTTTTAGAGCTAGAA |
| Hairpin1 | ATAGCAAGTTAAAATAAGGCTAGTCCGTTATCAACTTGGCACCGAGTCGGTGCTTTTT |
| +2 | TT |
| Delete | AGTAATAATACGACTCACTATAGGGGGCCACTAGGGACAGGATGTTTTAGAGCTAGAA |
| Hairpin2 | ATAGCAAGTTAAAATAAGGCTAGTCCGTTATCAACTTGAAAAAGTGGTCCTTTTTTTT |
| −0 | TTTTT |
| Delete | AGTAATAATACGACTCACTATAGGGGGCCACTAGGGACAGGATGTTTTAGAGCTAGAA |
| Hairpin2 | ATAGCAAGTTAAAATAAGGCTAGTCCGTTATCAACTTGAAAAAGTGGTCTTTTTTTTT |
| +1 | TTTT |
| Delete | AGTAATAATACGACTCACTATAGGGGGCCACTAGGGACAGGATGTTTTAGAGCTAGAA |
| Hairpin2 | ATAGCAAGTTAAAATAAGGCTAGTCCGTTATCAACTTGAAAAAGTGGCTTTTTTTTTT |
| +2 | TTT |
| Decrease | AGTAATAATACGACTCACTATAGGGGGCCACTAGGGACAGGATGTTTTAGAGCTAGAA |
| N-H1 | ATAGCAAGTTAAAATAAGGCTAGTCCGTCAACTTGAAAAAGTGGCACCGAGTCGGTGC |
| Spacer | TTTTTTT |
| −3 | |
| Decrease | AGTAATAATACGACTCACTATAGGGGGCCACTAGGGACAGGATGTTTTAGAGCTAGAA |
| N-H1 | ATAGCAAGTTAAAATAAGGCTAGTCCGTACAACTTGAAAAAGTGGCACCGAGTCGGTG |
| Spacer | CTTTTTTT |
| −2 | |
| Decrease | AGTAATAATACGACTCACTATAGGGGGCCACTAGGGACAGGATGTTTTAGAGCTAGAA |
| N-H1 | ATAGCAAGTTAAAATAAGGCTAGTCCGTATCAACTTGAAAAAGTGGCACCGAGTCGGT |
| Spacer | GCTTTTTTT |
| −1 | |
| Increase | AGTAATAATACGACTCACTATAGGGGGCCACTAGGGACAGGATGTTTTAGAGCTAGAA |
| N-H1 | ATAGCAAGTTAAAATAAGGCTAGTCCGTTATCGAACTTGAAAAAGTGGCACCGAGTCG |
| Spacer | GTGCTTTTTTT |
| +1 | |
| Increase | AGTAATAATACGACTCACTATAGGGGGCCACTAGGGACAGGATGTTTTAGAGCTAGAA |
| N-H1 | ATAGCAAGTTAAAATAAGGCTAGTCCGTTATCGCAACTTGAAAAAGTGGCACCGAGTC |
| Spacer | GGTGCTTTTTTT |
| +2 | |
| Increase | AGTAATAATACGACTCACTATAGGGGGCCACTAGGGACAGGATGTTTTAGAGCTAGAA |
| N-H1 | ATAGCAAGTTAAAATAAGGCTAGTCCGTTATCTGCAACTTGAAAAAGTGGCACCGAGT |
| Spacer | CGGTGCTTTTTTT |
| +3 | |
| Nexus | AGTAATAATACGACTCACTATAGGGGGCCACTAGGGACAGGATGTTTTAGAGCTAGAA |
| Loop | ATAGCAAGTTAAAATAAGGCTAGCAGTCCGTTATCAACTTGAAAAAGTGGCACCGAGT |
| | CGGTGCTTTTTTT |
| Nexus | AGTAATAATACGACTCACTATAGGGGGCCACTAGGGACAGGATGTTTTAGAGCTAGAA |
| Loop | ATAGCAAGTTAAAATAAGGCTATGACTAGTCCGTTATCAACTTGAAAAAGTGGCACCG |
| | AGTCGGTGCTTTTTTT |
| Nexus | AGTAATAATACGACTCACTATAGGGGGCCACTAGGGACAGGATGTTTTAGAGCTAGAA |
| Loop | ATAGCAAGTTAAAATAAGGCTACTGAAACAGGTCCGTTATCAACTTGAAAAAGTGGCA |
| | CCGAGTCGGTGCTTTTTTT |
| Nexus | AGTAATAATACGACTCACTATAGGGGGCCACTAGGGACAGGATGTTTTAGAGCTAGAA |
| Loop | ATAGCAAGTTAAAATAAGGCTAGGATTTCAATCCAAGTCCGTTATCAACTTGAAAAAG |
| | TGGCACCGAGTCGGTGCTTTTTTT |
| AAVS | AGTAATAATACGACTCACTATAGGGGGCCACTAGGGACAGGATGTCTCAGAGCTAGAA |
| wt_T7 | ATAGCAAGTTGAGATAAGGCTAGTCCGTTATCAACTTGAAAAAGTGGCACCGAGTCGG |
| | TGCTTTTTTT |
| AAVS | AGTAATAATACGACTCACTATAGGGGGCCACTAGGGACAGGATGTCTCAGAGCTAGAA |
| GNR | ATAGCAAGTTGAGATAAGGCTAGTCCGTTATCAACTTGCATAAGTGGCACCGAGTCGG |
| loop_T | TGCTTTTTTT |
| 7 | |
| AAVS | AGTAATAATACGACTCACTATAGGGGGCCACTAGGGACAGGATGTCTCAGAGCTAGAA |
| Csy4 | ATAGCAAGTTGAGATAAGGCTAGTCCGTTATCAACTTGTATAAAGTGGCACCGAGTCG |
| loop_T | GTGCTTTTTTT |
| 7 | |
| AAVS | AGTAATAATACGACTCACTATAGGGAGAGTTCACTGCCGTATAGGCAGGGGGCCACTA |
| Csy4 | GGGACAGGATGTCTCAGAGCTAGAAATAGCAAGTTGAGATAAGGCTAGTCCGTTATCA |
| GX19_T | ACTTGCATAAGTGGCACCGAGTCGGTGCTTTTTTT |
| 7 | |
| AAVS | AGTAATAATACGACTCACTATAGGGAGAGTTCACTGCCGTATAGGCAGAGGGCCACTA |
| Csy4 | GGGACAGGATGTCTCAGAGCTAGAAATAGCAAGTTGAGATAAGGCTAGTCCGTTATCA |
| AX19_T | ACTTGCATAAGTGGCACCGAGTCGGTGCTTTTTTT |
| 7 | |
| AAVS | AGTAATAATACGACTCACTATAGGGAGAGTTCACTGCCGTATAGGCAGTGGGCCACTA |
| Csy4 | GGGACAGGATGTCTCAGAGCTAGAAATAGCAAGTTGAGATAAGGCTAGTCCGTTATCA |
| TX19_T | ACTTGCATAAGTGGCACCGAGTCGGTGCTTTTTTT |
| 7 | |
| AAVS | AGTAATAATACGACTCACTATAGGGAGAGTTCACTGCCGTATAGGCAGCGGGCCACTA |
| Csy4 | GGGACAGGATGTCTCAGAGCTAGAAATAGCAAGTTGAGATAAGGCTAGTCCGTTATCA |
| CX19_T | ACTTGCATAAGTGGCACCGAGTCGGTGCTTTTTTT |
| 7 | |
| AAVS - | AGTAATAATACGACTCACTATAGGGGGCCACTAGGGACAGGATGTCTCAGAGCTAGAA |
| 1 bp | ATAGCAAGTTGAGATAAGGCTAGTCCGTTATCAACTGCATAGTGGCACCGAGTCGGTG |
| HP1_T7 | CTTTTTTT |
| AAVS - | AGTAATAATACGACTCACTATAGGGGGCCACTAGGGACAGGATGTCTCAGAGCTAGAA |
| 2 bp | ATAGCAAGTTGAGATAAGGCTAGTCCGTTATCAACTCATGTGGCACCGAGTCGGTGCT |
| HP1_T7 | TTTTTT |
| AAVS - | AGTAATAATACGACTCACTATAGGGGGCCACTAGGGACAGGATGTCTCAGAGCTAGAA |
| 4 bp | ATAGCAAGTTGAGATAAGGCTAGTCCGTTATCAACTATTGGCACCGAGTCGGTGCTTT |
| HP1_T7 | TTTT |
| AAVS | AGTAATAATACGACTCACTATAGGGGGCCACTAGGGACAGGATGTCTCAGAGCTAGAA |
| Delete | ATAGCAAGTTGAGATAAGGCTAGTCCGTTATCAACTTGGCACCGAGTCGGTGCTTTTT |
| HP1_T7 | TT |
| AAVS - | AGTAATAATACGACTCACTATAGGGGGCCACTAGGGACAGGATGTCTCAGAGCTAGAA |
| 2 bp | ATAGCAAGTTGAGATAAGGCTAGTCCGTTATCAACTTGCATAAGTGGCACAGTGTGCT |
| HP2_T7 | TTTTTT |
| AAVS - | AGTAATAATACGACTCACTATAGGGGGCCACTAGGGACAGGATGTCTCAGAGCTAGAA |
| 4 bp | ATAGCAAGTTGAGATAAGGCTAGTCCGTTATCAACTTGCATAAGTGGCAGTGCTTTTT |
| HP2_T7 | TT |
| AAVS - | AGTAATAATACGACTCACTATAGGGGGCCACTAGGGACAGGATGTCTCAGAGCTAGAA |
| 7 bp | ATAGCAAGTTGAGATAAGGCTAGTCCGTTATCAACTTGCATAAGTGAGTTTTTTTT |
| HP2_T7 | |
| AAVS | AGTAATAATACGACTCACTATAGGGGGCCACTAGGGACAGGATGTCTCAGAGCTAGAA |
| Delete | ATAGCAAGTTGAGATAAGGCTAGTCCGTTATCAACTTGCATAAGTGGTTTTTTT |
| HP2_T7 | |
| AAVS - | AGTAATAATACGACTCACTATAGGGGGCCACTAGGGACAGGATGTCTCAGAGTAGAAA |
| 1 bp | TACAAGTTGAGATAAGGCTAGTCCGTTATCAACTTGCATAAGTGCACCGAGTCGGTGC |
| Rp/Arp | TTTTTTT |
| _mid | |
| AAVS - | AGTAATAATACGACTCACTATAGGGGGCCACTAGGGACAGGATGTCTCAGAGAGAAAT |
| 2 bp | CAAGTTGAGATAAGGCTAGTCCGTTATCAACTTGCATAAGTGCACCGAGTCGGTGCTT |
| Rp/Arp | TTTTT |
| _mid | |
| AAVS - | AGTAATAATACGACTCACTATAGGGGGCCACTAGGGACAGGATGTCTCAGAGAGAATC |
| 3 bp | AAGTTGAGATAAGGCTAGTCCGTTATCAACTTGCATAAGTGCACCGAGTCGGTGCTTT |
| Rp/Arp | TTTT |
| _mid | |
| AAVS | AGTAATAATACGACTCACTATAGGGGGCCACTAGGGACAGGATGTCTCAGAGACAAGT |
| GV- | TGAGATAAGGCTAGTCCGTTATCAACTTGCATAAGTGCACCGAGTCGGTGCTTTTTTT |
| 11_mid | |
| AAVS - | AGTAATAATACGACTCACTATAGGGGGCCACTAGGGACAGGATGTCTCAGAGCTAGAA |
| 2 bp | ATAGCAAGTTGAGATAAGGCTAGTCCGTTATCAACTGCATAGTGGCACAGTGTGCTTT |
| HP1/HP | TTTT |
| 2_T7 | |
| AAVS - | AGTAATAATACGACTCACTATAGGGGGCCACTAGGGACAGGATGTCTCAGAGCTAGAA |
| 4 bp | ATAGCAAGTTGAGATAAGGCTAGTCCGTTATCAACTCATGTGGCAGTGCTTTTTTT |
| HP1/HP | |
| 2_T7 | |
| AAVS - | AGTAATAATACGACTCACTATAGGGGGCCACTAGGGACAGGATGTCTCAGAGCTAGAA |
| 7 bp | ATAGCAAGTTGAGATAAGGCTAGTCCGTTATCAACTATTGAGTTTTTTTT |
| HP1/HP | |
| 2_T7 | |
| AAVS | AGTAATAATACGACTCACTATAGGGGGCCACTAGGGACAGGATGTCTCAGAGCTAGAA |
| Delete | ATAGCAAGTTGAGATAAGGCTAGTCCGTTATCAACTTGGTTTTTTT |
| HP1/2_ | |
| T7 | |
| AAVS | AGTAATAATACGACTCACTATAGGGGGCCACTAGGGACAGGATGTCTCAGAGTAGAAA |
| miniGV | TACAAGTTGAGATAAGGCTAGTCCGTTATCAACTGCATAGTGCACCGAGTCGGTGCTT |
| −01_T7 | TTTTT |
| AAVS | AGTAATAATACGACTCACTATAGGGGGCCACTAGGGACAGGATGTCTCAGAGAGAAAT |
| miniGV | CAAGTTGAGATAAGGCTAGTCCGTTATCAACTCATGTGCACCGAGTCGGTGCTTTTTT |
| −02_T7 | T |
| AAVS | AGTAATAATACGACTCACTATAGGGGGCCACTAGGGACAGGATGTCTCAGAGAGAATC |
| miniGV | AAGTTGAGATAAGGCTAGTCCGTTATCAACTATTGCACCGAGTCGGTGCTTTTTTT |
| −03_T7 | |
| AAVS | AGTAATAATACGACTCACTATAGGGGGCCACTAGGGACAGGATGTCTCAGAGACAAGT |
| miniGV | TGAGATAAGGCTAGTCCGTTATCAACTTGCACCGAGTCGGTGCTTTTTTT |
| −04_T7 | |
| AAVS | AGTAATAATACGACTCACTATAGGGGGCCACTAGGGACAGGATGTCTCAGAGTAGAAA |
| miniGV | TACAAGTTGAGATAAGGCTAGTCCGTTATCAACTGCATAGTGGCACAGTGTGCTTTTT |
| −05_T7 | TT |
| AAVS | AGTAATAATACGACTCACTATAGGGGGCCACTAGGGACAGGATGTCTCAGAGAGAAAT |
| miniGV | CAAGTTGAGATAAGGCTAGTCCGTTATCAACTCATGTGGCAGTGCTTTTTTT |
| −06_T7 | |
| AAVS | AGTAATAATACGACTCACTATAGGGGGCCACTAGGGACAGGATGTCTCAGAGAGAATC |
| miniGV | AAGTTGAGATAAGGCTAGTCCGTTATCAACTATTGAGTTTTTTTT |
| −07_T7 | |
| AAVS | AGTAATAATACGACTCACTATAGGGGGCCACTAGGGACAGGATGTCTCAGAGACAAGT |
| miniGV | TGAGATAAGGCTAGTCCGTTATCAACTTGGTTTTTTT |
| −08_T7 | |
| EMX | AGTAATAATACGACTCACTATAGGAGTCCGAGCAGAAGAAGAAGTCTCAGAGCTAGAA |
| wt_T7 | ATAGCAAGTTGAGATAAGGCTAGTCCGTTATCAACTTGAAAAAGTGGCACCGAGTCGG |
| | TGCTTTTTTT |
| EMX | AGTAATAATACGACTCACTATAGGAGTCCGAGCAGAAGAAGAAGTCTCAGAGCTAGAA |
| GNR | ATAGCAAGTTGAGATAAGGCTAGTCCGTTATCAACTTGCATAAGTGGCACCGAGTCGG |
| loop_T | TGCTTTTTTT |
| 7 | |
| EMX | AGTAATAATACGACTCACTATAGGAGTCCGAGCAGAAGAAGAAGTCTCAGAGCTAGAA |
| Csy4 | ATAGCAAGTTGAGATAAGGCTAGTCCGTTATCAACTTGTATAAAGTGGCACCGAGTCG |
| loop_T | GTGCTTTTTTT |
| 7 | |
| EMX | AGTAATAATACGACTCACTATAGGGAGAGTTCACTGCCGTATAGGCAGGAGTCCGAGC |
| Csy4 | AGAAGAAGAAGTCTCAGAGCTAGAAATAGCAAGTTGAGATAAGGCTAGTCCGTTATCA |
| GX19_T | ACTTGCATAAGTGGCACCGAGTCGGTGCTTTTTTT |
| 7 | |
| EMX | AGTAATAATACGACTCACTATAGGGAGAGTTCACTGCCGTATAGGCAGAAGTCCGAGC |
| Csy4 | AGAAGAAGAAGTCTCAGAGCTAGAAATAGCAAGTTGAGATAAGGCTAGTCCGTTATCA |
| AX19_T | ACTTGCATAAGTGGCACCGAGTCGGTGCTTTTTTT |
| 7 | |
| EMX | AGTAATAATACGACTCACTATAGGGAGAGTTCACTGCCGTATAGGCAGTAGTCCGAGC |
| Csy4 | AGAAGAAGAAGTCTCAGAGCTAGAAATAGCAAGTTGAGATAAGGCTAGTCCGTTATCA |
| TX19_T | ACTTGCATAAGTGGCACCGAGTCGGTGCTTTTTTT |
| 7 | |
| EMX | AGTAATAATACGACTCACTATAGGGAGAGTTCACTGCCGTATAGGCAGCAGTCCGAGC |
| Csy4 | AGAAGAAGAAGTCTCAGAGCTAGAAATAGCAAGTTGAGATAAGGCTAGTCCGTTATCA |
| CX19_T | ACTTGCATAAGTGGCACCGAGTCGGTGCTTTTTTT |
| 7 | |
| EMX - | AGTAATAATACGACTCACTATAGGAGTCCGAGCAGAAGAAGAAGTCTCAGAGCTAGAA |
| 1 bp | ATAGCAAGTTGAGATAAGGCTAGTCCGTTATCAACTGCATAGTGGCACCGAGTCGGTG |
| HP1_T7 | CTTTTTTT |
| EMX - | AGTAATAATACGACTCACTATAGGAGTCCGAGCAGAAGAAGAAGTCTCAGAGCTAGAA |
| 2 bp | ATAGCAAGTTGAGATAAGGCTAGTCCGTTATCAACTCATGTGGCACCGAGTCGGTGCT |
| HP1_T7 | TTTTTT |
| EMX - | AGTAATAATACGACTCACTATAGGAGTCCGAGCAGAAGAAGAAGTCTCAGAGCTAGAA |
| 4 bp | ATAGCAAGTTGAGATAAGGCTAGTCCGTTATCAACTATTGGCACCGAGTCGGTGCTTT |
| HP1_T7 | TTTT |
| EMX | AGTAATAATACGACTCACTATAGGAGTCCGAGCAGAAGAAGAAGTCTCAGAGCTAGAA |
| Delete | ATAGCAAGTTGAGATAAGGCTAGTCCGTTATCAACTTGGCACCGAGTCGGTGCTTTTT |
| HP1_T7 | TT |
| EMX - | AGTAATAATACGACTCACTATAGGAGTCCGAGCAGAAGAAGAAGTCTCAGAGCTAGAA |
| 2 bp | ATAGCAAGTTGAGATAAGGCTAGTCCGTTATCAACTTGCATAAGTGGCACAGTGTGCT |
| HP2_T7 | TTTTTT |
| EMX - | AGTAATAATACGACTCACTATAGGAGTCCGAGCAGAAGAAGAAGTCTCAGAGCTAGAA |
| 4 bp | ATAGCAAGTTGAGATAAGGCTAGTCCGTTATCAACTTGCATAAGTGGCAGTGCTTTTT |
| HP2_T7 | TT |
| EMX - | AGTAATAATACGACTCACTATAGGAGTCCGAGCAGAAGAAGAAGTCTCAGAGCTAGAA |
| 7 bp | ATAGCAAGTTGAGATAAGGCTAGTCCGTTATCAACTTGCATAAGTGAGTTTTTTTT |
| HP2_T7 | |
| EMX | AGTAATAATACGACTCACTATAGGAGTCCGAGCAGAAGAAGAAGTCTCAGAGCTAGAA |
| Delete | ATAGCAAGTTGAGATAAGGCTAGTCCGTTATCAACTTGCATAAGTGGTTTTTTT |
| HP2_T7 | |
| EMX - | AGTAATAATACGACTCACTATAGGAGTCCGAGCAGAAGAAGAAGTCTCAGAGTAGAAA |
| 1 bp | TACAAGTTGAGATAAGGCTAGTCCGTTATCAACTTGCATAAGTGCACCGAGTCGGTGC |
| Rp/Arp | TTTTTTT |
| _T7 | |
| EMX - | AGTAATAATACGACTCACTATAGGAGTCCGAGCAGAAGAAGAAGTCTCAGAGAGAAAT |
| 2 bp | CAAGTTGAGATAAGGCTAGTCCGTTATCAACTTGCATAAGTGCACCGAGTCGGTGCTT |
| Rp/Arp | TTTTT |
| _T7 | |
| EMX - | AGTAATAATACGACTCACTATAGGAGTCCGAGCAGAAGAAGAAGTCTCAGAGAGAATC |
| 3 bp | AAGTTGAGATAAGGCTAGTCCGTTATCAACTTGCATAAGTGCACCGAGTCGGTGCTTT |
| Rp/Arp | TTTT |
| _T7 | |
| EMX | AGTAATAATACGACTCACTATAGGAGTCCGAGCAGAAGAAGAAGTCTCAGAGACAAGT |
| GV- | TGAGATAAGGCTAGTCCGTTATCAACTTGCATAAGTGCACCGAGTCGGTGCTTTTTTT |
| 11_T7 | |
| EMX - | AGTAATAATACGACTCACTATAGGAGTCCGAGCAGAAGAAGAAGTCTCAGAGCTAGAA |
| 2 bp | ATAGCAAGTTGAGATAAGGCTAGTCCGTTATCAACTGCATAGTGGCACAGTGTGCTTT |
| HP1/HP | TTTT |
| 2_T7 | |
| EMX - | AGTAATAATACGACTCACTATAGGAGTCCGAGCAGAAGAAGAAGTCTCAGAGCTAGAA |
| 4 bp | ATAGCAAGTTGAGATAAGGCTAGTCCGTTATCAACTCATGTGGCAGTGCTTTTTTT |
| HP1/HP | |
| 2_T7 | |
| EMX - | AGTAATAATACGACTCACTATAGGAGTCCGAGCAGAAGAAGAAGTCTCAGAGCTAGAA |
| 7 bp | ATAGCAAGTTGAGATAAGGCTAGTCCGTTATCAACTATTGAGTTTTTTTT |
| HP1/HP | |
| 2_T7 | |
| EMX | AGTAATAATACGACTCACTATAGGAGTCCGAGCAGAAGAAGAAGTCTCAGAGCTAGAA |
| Delete | ATAGCAAGTTGAGATAAGGCTAGTCCGTTATCAACTTGGTTTTTTT |
| HP1/2_ | |
| T7 | |
| EMX | AGTAATAATACGACTCACTATAGGAGTCCGAGCAGAAGAAGAAGTCTCAGAGTAGAAA |
| miniGV | TACAAGTTGAGATAAGGCTAGTCCGTTATCAACTGCATAGTGCACCGAGTCGGTGCTT |
| −01_T7 | TTTTT |
| EMX | AGTAATAATACGACTCACTATAGGAGTCCGAGCAGAAGAAGAAGTCTCAGAGAGAAAT |
| miniGV | CAAGTTGAGATAAGGCTAGTCCGTTATCAACTCATGTGCACCGAGTCGGTGCTTTTTT |
| −02_T7 | T |
| EMX | AGTAATAATACGACTCACTATAGGAGTCCGAGCAGAAGAAGAAGTCTCAGAGAGAATC |
| miniGV | AAGTTGAGATAAGGCTAGTCCGTTATCAACTATTGCACCGAGTCGGTGCTTTTTTT |
| −03_T7 | |
| EMX | AGTAATAATACGACTCACTATAGGAGTCCGAGCAGAAGAAGAAGTCTCAGAGACAAGT |
| miniGV | TGAGATAAGGCTAGTCCGTTATCAACTTGCACCGAGTCGGTGCTTTTTTT |
| −04_T7 | |
| EMX | AGTAATAATACGACTCACTATAGGAGTCCGAGCAGAAGAAGAAGTCTCAGAGTAGAAA |
| miniGV | TACAAGTTGAGATAAGGCTAGTCCGTTATCAACTGCATAGTGGCACAGTGTGCTTTTT |
| −05_T7 | TT |
| EMX | AGTAATAATACGACTCACTATAGGAGTCCGAGCAGAAGAAGAAGTCTCAGAGAGAAAT |
| miniGV | CAAGTTGAGATAAGGCTAGTCCGTTATCAACTCATGTGGCAGTGCTTTTTTT |
| −06_T7 | |
| EMX | AGTAATAATACGACTCACTATAGGAGTCCGAGCAGAAGAAGAAGTCTCAGAGAGAATC |
| miniGV | AAGTTGAGATAAGGCTAGTCCGTTATCAACTATTGAGTTTTTTTT |
| −07_T7 | |
| EMX | AGTAATAATACGACTCACTATAGGAGTCCGAGCAGAAGAAGAAGTCTCAGAGACAAGT |
| miniGV | TGAGATAAGGCTAGTCCGTTATCAACTTGGTTTTTTT |
| −08_T7 | |
| VEGFA | AGTAATAATACGACTCACTATAGGGGTGGGGGGAGTTTGCTCCGTCTCAGAGCTAGAA |
| wt_T7 | ATAGCAAGTTGAGATAAGGCTAGTCCGTTATCAACTTGAAAAAGTGGCACCGAGTCGG |
| | TGCTTTTTTT |
| VEGFA | AGTAATAATACGACTCACTATAGGGGTGGGGGGAGTTTGCTCCGTCTCAGAGCTAGAA |
| GNR | ATAGCAAGTTGAGATAAGGCTAGTCCGTTATCAACTTGCATAAGTGGCACCGAGTCGG |
| loop_T | TGCTTTTTTT |
| 7 | |
| VEGFA | AGTAATAATACGACTCACTATAGGGGTGGGGGGAGTTTGCTCCGTCTCAGAGCTAGAA |
| Csy4 | ATAGCAAGTTGAGATAAGGCTAGTCCGTTATCAACTTGTATAAAGTGGCACCGAGTCG |
| loop_T | GTGCTTTTTTT |
| 7 | |
| VEGFA | AGTAATAATACGACTCACTATAGGGAGAGTTCACTGCCGTATAGGCAGGGGTGGGGGG |
| Csy4 | AGTTTGCTCCGTCTCAGAGCTAGAAATAGCAAGTTGAGATAAGGCTAGTCCGTTATCA |
| GX19_T | ACTTGCATAAGTGGCACCGAGTCGGTGCTTTTTTT |
| 7 | |
| VEGFA | AGTAATAATACGACTCACTATAGGGAGAGTTCACTGCCGTATAGGCAGAGGTGGGGGG |
| Csy4 | AGTTTGCTCCGTCTCAGAGCTAGAAATAGCAAGTTGAGATAAGGCTAGTCCGTTATCA |
| AX19_T | ACTTGCATAAGTGGCACCGAGTCGGTGCTTTTTTT |
| 7 | |
| VEGFA | AGTAATAATACGACTCACTATAGGGAGAGTTCACTGCCGTATAGGCAGTGGTGGGGGG |
| Csy4 | AGTTTGCTCCGTCTCAGAGCTAGAAATAGCAAGTTGAGATAAGGCTAGTCCGTTATCA |
| TX19_T | ACTTGCATAAGTGGCACCGAGTCGGTGCTTTTTTT |
| 7 | |
| VEGFA | AGTAATAATACGACTCACTATAGGGAGAGTTCACTGCCGTATAGGCAGCGGTGGGGGG |
| Csy4 | AGTTTGCTCCGTCTCAGAGCTAGAAATAGCAAGTTGAGATAAGGCTAGTCCGTTATCA |
| CX19_T | ACTTGCATAAGTGGCACCGAGTCGGTGCTTTTTTT |
| 7 | |
| VEGFA | AGTAATAATACGACTCACTATAGGGGTGGGGGGAGTTTGCTCCGTCTCAGAGCTAGAA |
| −1 bp | ATAGCAAGTTGAGATAAGGCTAGTCCGTTATCAACTGCATAGTGGCACCGAGTCGGTG |
| HP1_T7 | CTTTTTTT |
| VEGFA | AGTAATAATACGACTCACTATAGGGGTGGGGGGAGTTTGCTCCGTCTCAGAGCTAGAA |
| −2 bp | ATAGCAAGTTGAGATAAGGCTAGTCCGTTATCAACTCATGTGGCACCGAGTCGGTGCT |
| HP1_T7 | TTTTTT |
| VEGFA | AGTAATAATACGACTCACTATAGGGGTGGGGGGAGTTTGCTCCGTCTCAGAGCTAGAA |
| −4 bp | ATAGCAAGTTGAGATAAGGCTAGTCCGTTATCAACTATTGGCACCGAGTCGGTGCTTT |
| HP1_T7 | TTTT |
| VEGFA | AGTAATAATACGACTCACTATAGGGGTGGGGGGAGTTTGCTCCGTCTCAGAGCTAGAA |
| Delete | ATAGCAAGTTGAGATAAGGCTAGTCCGTTATCAACTTGGCACCGAGTCGGTGCTTTTT |
| HP1_T7 | TT |
| VEGFA | AGTAATAATACGACTCACTATAGGGGTGGGGGGAGTTTGCTCCGTCTCAGAGCTAGAA |
| −2 bp | ATAGCAAGTTGAGATAAGGCTAGTCCGTTATCAACTTGCATAAGTGGCACAGTGTGCT |
| HP2_T7 | TTTTTT |
| VEGFA | AGTAATAATACGACTCACTATAGGGGTGGGGGGAGTTTGCTCCGTCTCAGAGCTAGAA |
| −4 bp | ATAGCAAGTTGAGATAAGGCTAGTCCGTTATCAACTTGCATAAGTGGCAGTGCTTTTT |
| HP2_T7 | TT |
| VEGFA | AGTAATAATACGACTCACTATAGGGGTGGGGGGAGTTTGCTCCGTCTCAGAGCTAGAA |
| −7 bp | ATAGCAAGTTGAGATAAGGCTAGTCCGTTATCAACTTGCATAAGTGAGTTTTTTTT |
| HP2_T7 | |
| VEGFA | AGTAATAATACGACTCACTATAGGGGTGGGGGGAGTTTGCTCCGTCTCAGAGCTAGAA |
| Delete | ATAGCAAGTTGAGATAAGGCTAGTCCGTTATCAACTTGCATAAGTGGTTTTTTT |
| HP2_T7 | |
| VEGFA | AGTAATAATACGACTCACTATAGGGGTGGGGGGAGTTTGCTCCGTCTCAGAGTAGAAA |
| −1 bp | TACAAGTTGAGATAAGGCTAGTCCGTTATCAACTTGCATAAGTGCACCGAGTCGGTGC |
| Rp/Arp | TTTTTTT |
| _T7 | |
| VEGFA | AGTAATAATACGACTCACTATAGGGGTGGGGGGAGTTTGCTCCGTCTCAGAGAGAAAT |
| −2 bp | CAAGTTGAGATAAGGCTAGTCCGTTATCAACTTGCATAAGTGCACCGAGTCGGTGCTT |
| Rp/Arp | TTTTT |
| _T7 | |
| VEGFA | AGTAATAATACGACTCACTATAGGGGTGGGGGGAGTTTGCTCCGTCTCAGAGAGAATC |
| −3 bp | AAGTTGAGATAAGGCTAGTCCGTTATCAACTTGCATAAGTGCACCGAGTCGGTGCTTT |
| Rp/Arp | TTTT |
| _T7 | |
| VEGFA | AGTAATAATACGACTCACTATAGGGGTGGGGGGAGTTTGCTCCGTCTCAGAGACAAGT |
| GV- | TGAGATAAGGCTAGTCCGTTATCAACTTGCATAAGTGCACCGAGTCGGTGCTTTTTTT |
| 11_T7 | |
| VEGFA | AGTAATAATACGACTCACTATAGGGGTGGGGGGAGTTTGCTCCGTCTCAGAGCTAGAA |
| −2 bp | ATAGCAAGTTGAGATAAGGCTAGTCCGTTATCAACTGCATAGTGGCACAGTGTGCTTT |
| HP1/HP | TTTT |
| 2_T7 | |
| VEGFA | AGTAATAATACGACTCACTATAGGGGTGGGGGGAGTTTGCTCCGTCTCAGAGCTAGAA |
| −4 bp | ATAGCAAGTTGAGATAAGGCTAGTCCGTTATCAACTCATGTGGCAGTGCTTTTTTT |
| HP1/HP | |
| 2_T7 | |
| VEGFA | AGTAATAATACGACTCACTATAGGGGTGGGGGGAGTTTGCTCCGTCTCAGAGCTAGAA |
| −7 bp | ATAGCAAGTTGAGATAAGGCTAGTCCGTTATCAACTATTGAGTTTTTTTT |
| HP1/HP | |
| 2_T7 | |
| VEGFA | AGTAATAATACGACTCACTATAGGGGTGGGGGGAGTTTGCTCCGTCTCAGAGCTAGAA |
| Delete | ATAGCAAGTTGAGATAAGGCTAGTCCGTTATCAACTTGGTTTTTTT |
| HP1/2_ | |
| T7 | |
| VEGFA | AGTAATAATACGACTCACTATAGGGGTGGGGGGAGTTTGCTCCGTCTCAGAGTAGAAA |
| miniGV | TACAAGTTGAGATAAGGCTAGTCCGTTATCAACTGCATAGTGCACCGAGTCGGTGCTT |
| −01_T7 | TTTTT |
| VEGFA | AGTAATAATACGACTCACTATAGGGGTGGGGGGAGTTTGCTCCGTCTCAGAGAGAAAT |
| miniGV | CAAGTTGAGATAAGGCTAGTCCGTTATCAACTCATGTGCACCGAGTCGGTGCTTTTTT |
| −02_T7 | T |
| VEGFA | AGTAATAATACGACTCACTATAGGGGTGGGGGGAGTTTGCTCCGTCTCAGAGAGAATC |
| miniGV | AAGTTGAGATAAGGCTAGTCCGTTATCAACTATTGCACCGAGTCGGTGCTTTTTTT |
| −03_T7 | |
| VEGFA | AGTAATAATACGACTCACTATAGGGGTGGGGGGAGTTTGCTCCGTCTCAGAGACAAGT |
| miniGV | TGAGATAAGGCTAGTCCGTTATCAACTTGCACCGAGTCGGTGCTTTTTTT |
| −04_T7 | |
| VEGFA | AGTAATAATACGACTCACTATAGGGGTGGGGGGAGTTTGCTCCGTCTCAGAGTAGAAA |
| miniGV | TACAAGTTGAGATAAGGCTAGTCCGTTATCAACTGCATAGTGGCACAGTGTGCTTTTT |
| −05_T7 | TT |
| VEGFA | AGTAATAATACGACTCACTATAGGGGTGGGGGGAGTTTGCTCCGTCTCAGAGAGAAAT |
| miniGV | CAAGTTGAGATAAGGCTAGTCCGTTATCAACTCATGTGGCAGTGCTTTTTTT |
| −06_T7 | |
| VEGFA | AGTAATAATACGACTCACTATAGGGGTGGGGGGAGTTTGCTCCGTCTCAGAGAGAATC |
| miniGV | AAGTTGAGATAAGGCTAGTCCGTTATCAACTATTGAGTTTTTTTT |
| −07_T7 | |
| VEGFA | AGTAATAATACGACTCACTATAGGGGTGGGGGGAGTTTGCTCCGTCTCAGAGACAAGT |
| miniGV | TGAGATAAGGCTAGTCCGTTATCAACTTGGTTTTTTT |
| −08_T7 | |

What is claimed is:

1. A composition comprising:
(a) a genomic DNA, wherein said genomic DNA comprises a target region;
(b) a Cas9 polypeptide; and
(c) a engineered nucleic acid-targeting nucleic acid comprising
(i) a stem loop duplex structure,
(ii) a spacer located 5′ to said stem loop duplex structure, and
(iii) a spacer extension located 5′ to said spacer,
wherein said Cas9 polypeptide and said engineered nucleic acid-targeting nucleic acid have a decreased ability to modify a genomic DNA region that is not said target region as compared to said Cas9 polypeptide and a control nucleic acid-targeting nucleic acid that does not comprise a spacer extension.

2. A composition comprising a cell modified with the composition of claim 1.

3. The composition of claim 2, wherein said cell comprises a eukaryotic cell.

4. The composition of claim 3, wherein said cell comprises a stem cell.

5. The composition of claim 1, wherein said engineered nucleic acid-targeting nucleic acid comprises RNA nucleobases.

6. The composition of claim 1, wherein said engineered nucleic acid-targeting nucleic acid is RNA.

7. The composition of claim 1, wherein said engineered nucleic acid-targeting nucleic acid comprises modified nucleobases.

8. The composition of claim 1, wherein said engineered nucleic acid-targeting nucleic acid further comprises a covalently linked moiety.

9. The composition of claim 1, wherein said engineered nucleic acid-targeting nucleic acid comprises one or more mutations.

10. The composition of claim 9, wherein said one or more mutations comprises an insertion of one or more nucleotides.

11. The composition of claim 9, wherein said one or more mutation comprises a deletion of one or more nucleotides.

12. The composition of claim 9, wherein said one or more mutations comprises a substitution of one or more nucleotides with a modified nucleotide.

13. The composition of claim 1, wherein said engineered nucleic acid-targeting nucleic acid comprises two or more mutations and a first mutation is separated by at least one nucleobase from a second mutation.

14. The composition of claim 1, wherein said engineered nucleic acid-targeting nucleic acid comprises two or more mutations and a first mutation is adjacent to a second mutation.

15. The composition of claim 1, wherein said Cas9 polypeptide and said engineered nucleic acid-targeting nucleic acid have about a 10% decreased ability to modify a genomic DNA region that is not said target region as compared to said Cas9 polypeptide and a control nucleic acid-targeting nucleic acid that does not comprise a spacer extension.

16. The composition of claim 1, wherein said spacer region comprises between 18 to 21 nucleotides in length, inclusive.

17. The composition of claim 1, wherein said modification of the target region of the genomic DNA comprises cleavage of a phosphodiester bond.

18. The composition of claim 1, wherein said spacer extension comprises a G.

19. The composition of claim 1, wherein said spacer extension comprises an A.

20. The composition of claim 1, wherein said spacer extension comprises an U.

21. The composition of claim 1, wherein said spacer extension comprises a C.

22. The composition of claim 1, wherein said spacer extension comprises one or more additional 5′ nucleotides.

23. The composition of claim 22, wherein said one or more additional 5′ nucleotides is a G.

24. The composition of claim 1, wherein a combined length of said spacer extension and said spacer region is between 20 to 22 nucleotides in length, inclusive.

25. The composition of claim 1, wherein the engineered nucleic acid-targeting nucleic acid further comprises an engineered region selected from the group consisting of: an engineered bulge region, an engineered hairpin located 3′ of the stem loop duplex structure, and any combination thereof.

26. The composition of claim 25, wherein said engineered nucleic acid-targeting nucleic acid comprises one or more mutations in said engineered region selected from the group consisting of: an engineered bulge region, an engineered hairpin located 3′ of the stem loop duplex structure, and any combination thereof.

27. A kit comprising:
(a) the composition of claim 1; and
(b) a suitable buffer.

28. The kit of claim 27, further comprising instructions for use.

29. A pharmaceutical composition comprising the cell of claim 2.

30. The pharmaceutical composition of claim 29, further comprising an excipient.
